(19) United States
(12) Patent Application Publication
(10) Pub. No.: US 2025/0051337 A1
(43) Pub. Date: Feb. 13, 2025

(54) INHIBITORS OF SERINE/THREONINE PROTEIN KINASE STK3 OR STK4 AND USES THEREOF

(71) Applicant: Sanford Burnham Prebys Medical Discovery Institute, La Jolla, CA (US)

(72) Inventors: Nicholas David Peter COSFORD, San Diego, CA (US); Nicole A. BAKAS, Oceanside, CA (US); Nicole BATA, San Diego, CA (US)

(21) Appl. No.: 18/696,213

(22) PCT Filed: Sep. 27, 2022
(86) PCT No.: PCT/US2022/044889

§ 371 Date: Mar. 27, 2024

Related U.S. Application Data
(60) Provisional application No. 63/249,316, filed on Sep. 28, 2021.

(51) Int. Cl. C07D 487/04; A61K 31/519; A61K 31/5377; C07D 471/04; C07D 491/048; C07D 495/04; C07D 519/00

(52) U.S. Cl. CPC C07D 487/04; A61K 31/519; A61K 31/5377; C07D 471/04; C07D 491/048; C07D 495/04; C07D 519/00

(57) ABSTRACT

Described herein are compounds capable of modulating the level of activity of Serine/threonine protein kinase 3 (STK3) or serine/threonine protein kinase 4 (STK4), compositions, and methods of using these compounds and compositions.

### CROSS-REFERENCE TO RELATED APPLICATIONS

This application claims the benefit of U.S. Provisional Application No. 63/249,316 filed on Sep. 28, 2021, which is hereby incorporated by reference in its entirety.

### FIELD OF THE INVENTION

Described herein are inhibitors of serine/threonine protein kinase 3 or 4 (STK3) or (STK4), also referred to as MST2 and MST1 respectively, methods of making such compounds, pharmaceutical compositions and medicaments comprising such compounds, and methods of using such compounds in the treatment of conditions, diseases, or disorders associated with STK 3 or STK 4 activity.

### BACKGROUND OF THE INVENTION

Acute myeloid leukemia (AML) is an umbrella term for a diverse collection of hematological cancers characterized by excessive production of immature myeloid cells in the bone marrow. Recently, a large-scale loss-of-function RNAi screen of patient-derived AML cells identified STK3 as a potential target for antileukemia therapy. Indeed, STK3 depletion induces cell death in some human AML cell lines and primary cells, and genetic inactivation of STK4 in multiple myeloma cells decreases their proliferation and induces a robust apoptotic response through YAP1 both in vitro and in vivo. STK3/4 have also been shown to directly regulate the autophagy pathway. Together, these data indicate that STK3/4 are antileukemia targets.

### BRIEF SUMMARY OF THE INVENTION

Described herein are compounds, pharmaceutical compositions and methods of modulating the activity of serine/threonine protein kinase 3 (STK3) or serine/threonine protein kinase 4 (STK4), or both.

Also disclosed herein is a compound of Formula (I), or a pharmaceutically acceptable salt thereof:

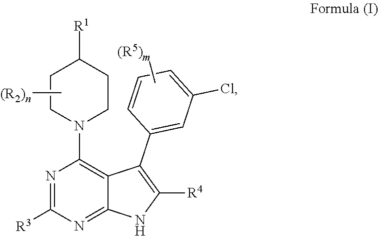

wherein:
R¹ is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{1a};
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{1a} on the same atom are taken together to form an oxo;
each R₂ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
n is 0-6;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl.

Also disclosed herein is a compound of Formula (II), or a pharmaceutically acceptable salt thereof:

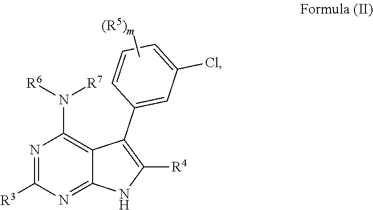

wherein:
R⁶ is hydrogen, C₁-C₆alkyl, C₁-C₆haloalkyl, C₁-C₆hydroxyalkyl, C₁-C₆aminoalkyl, or C₁-C₆heteroalkyl;
R⁷ is C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl; wherein the alkyl, alkenyl, and alkynyl is optionally and independently substituted with one or more R^{7a};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{7b};
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{7b} on the same atom are taken together to form an oxo;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O); NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
   provided that the compound is not:

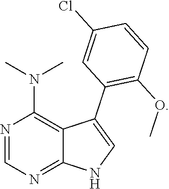 [SMILES: COc1ccc(Cl)cc1-c1c[nH]c2ncnc(N(C)C)c12]

Also disclosed herein is a compound of Formula (III), or a pharmaceutically acceptable salt thereof:

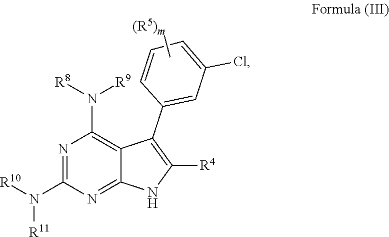

wherein:
R⁸ is hydrogen, C₁-C₆alkyl, C₁-C₆haloalkyl, C₁-C₆hydroxyalkyl, C₁-C₆aminoalkyl, or C₁-C₆heteroalkyl;
R⁹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the cycloalkyl, heterocycloalkyl, aryl, and heteroaryl is optionally and independently substituted with one or more R^{9b};
or two R^{9a} on the same atom are taken together to form an oxo;
each R^{9b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{9b} on the same atom are taken together to form an oxo;
or R⁸ and R⁹ are taken together with the atom to which they are attached to form a heterocycloalkyl optionally substituted with one or more R^{8a};
each R^{8a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{8a} on the same atom are taken together to form an oxo;
R¹⁰ is hydrogen, C₁-C₆alkyl, or C₁-C₆haloalkyl;
R¹¹ is C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{11a};
each R^{11a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d} C₁-C₆alkyl, C₁-C₆haloalkyl, C₁-C₆hydroxyalkyl, C₁-C₆aminoalkyl, or C₁-C₆heteroalkyl;
or two R^{11a} on the same atom are taken together to form an oxo;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl.

Also disclosed herein is a pharmaceutical composition comprising a compound disclosed herein, or a pharmaceutically acceptable salt thereof, and a pharmaceutically acceptable excipient.

Also disclosed herein is a method of modulating the activity serine/threonine protein kinase 3 (STK3) or serine/threonine protein kinase 4 (STK4) or both in a mammal, comprising administering to the mammal a compound disclosed herein, or a pharmaceutically acceptable salt thereof. In some embodiments, modulating comprises inhibiting. In some embodiments, the mammal has a disease or condition that would benefit from inhibition of STK3 or STK4 or both. In some embodiments, the disease or condition is cancer. In some embodiments, the cancer is a blood cancer. In some embodiments, the cancer is leukemia, lymphoma, myeloma, myeloproliferative neoplasms (MPNs), or myelodysplastic syndromes (MDS). In some embodiments, the leukemia is acute Lymphoblastic Leukemia (ALL), acute myeloid leukemia (AML), chronic lymphocytic leukemia (CLL), chronic myeloid leukemia (CML), hairy cell leukemia (HCL), chronic myelomonocytic leukemia (CMML), large granular lymphocytic (LGL), blastic plasmacytoid dendritic cell neoplasm (BPDCN), B-cell prolymphocytic leukemia (B-PLL), or T-cell prolymphocytic leukemia (T-PLL). In some embodiments, the lymphoma is Hodgkin Lymphoma (HL) or Non-Hodgkin Lymphoma (NHL). In some embodiments, the myeloma is multiple myeloma or plasmacytoma. In some embodiments, the myeloproliferative neoplasms is myelofibrosis, polycythemia vera or essential thrombocythemia.

Also disclosed herein is a method of modulating the activity serine/threonine protein kinase 3 (STK3) or serine/threonine protein kinase 4 (STK4) or both in a mammal, comprising administering to the mammal a compound of Formula (A), or a pharmaceutically acceptable salt thereof:

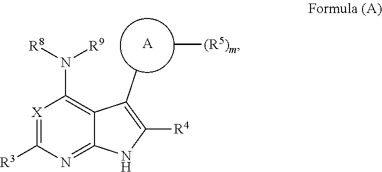

wherein:
X is N or CR^{X};
R^{X} is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
Ring A is aryl or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
R⁸ is hydrogen, C₁-C₆alkyl, C₁-C₆haloalkyl, C₁-C₆hydroxyalkyl, C₁-C₆aminoalkyl, or C₁-C₆heteroalkyl;
R⁹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
each R^{9a} is independently halogen, —CN, —NO₂, —OH, —OR^{a}, —OC(═O)R^{a}, —OC(═O)OR^{b}, —OC(═O)NR^{c}R^{d}, —SH, SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the cycloalkyl, heterocycloalkyl, aryl, and heteroaryl is optionally and independently substituted with one or more R^{9b};
or two R^{9a} on the same atom are taken together to form an oxo;
each R^{9b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{9b} on the same atom are taken together to form an oxo;
or R⁸ and R⁹ are taken together with the atom to which they are attached to form a heterocycloalkyl optionally substituted with one or more R^{8a};
each R^{8a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
or two R^{8a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
   provided that the compound is not

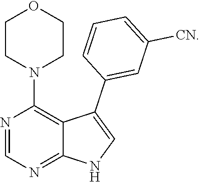 [SMILES: N#Cc1cccc(-c2c[nH]c3ncnc(N4CCOCC4)c23)c1]

### BRIEF DESCRIPTION OF THE DRAWINGS

FIG. 1A shows the binding affinities and thermodynamic signatures from isothermal titration calorimetry (ITC) for compounds 6 and 23.

FIG. 1B shows representative ITC binding data for the interactions between compound 6 and STK3. The normalized heat of binding with the single-site binding fits (red line) is shown.

FIG. 1C shows representative ITC binding data for the interactions between compound 6 and STK4. The normalized heat of binding with the single-site binding fits (red line) is shown.

FIG. 2A displays Western blot densitometry of phosphorylated (p)-MOB1 levels in HEK293 cells treated with DMSO (−) or 10 μM 6, 7, 9, 11, 15, 16, 106, 105, 108, 119, 121, or 23 for 4 h followed by treatment with H₂O₂ (50 μM) for an additional 2 h. Protein levels were quantified by densitometry using ImageJ software. Data are presented as pMOB1 levels relative to the DMSO-treated cells (n=3); ns>0.033, * p≤0.033, ** p≤0.002, *** p≤0.001 by one-way ANOVA with Fisher's LSD multiple comparison test

FIG. 2B displays luciferase reporter assay of HEK293 cells transfected with 8×GTIIC-firefly luciferase plasmid (YAP/TAZ-TEAD-responsive promoter) and control Renilla luciferase plasmid. Cells were treated with DMSO (−) or 10 μM 6, 7, 11, 15, 16, or 23 for 48 h. Data are presented as normalized (firefly: Renilla) luciferase activity relative to the DMSO-treated cells (n=3); ns>0.033, * p≤0.033, ** p<0.002, *** p≤0.001 by one-way ANOVA with Fisher's LSD multiple comparison test.

FIG. 2C displays the Western blot of p-MOB1. MOB1, and GAPDH levels in liver lysates from adult female C₅₇BL/6 mice injected intraperitoneally with vehicle and compound 105 (20 mg/kg) for 1 h.

FIG. 2D displays the Western blot of p-MOB1. MOB1, and GAPDH levels in liver lysates from adult female C₅₇BL/6 mice injected intraperitoneally with vehicle and compound 11 (20 mg/kg) for 1 h.

FIG. 2E displays Western blot densitometry of p-MOB1 levels from FIG. 2C and FIG. 2D. Protein levels were quantified by densitometry using ImageJ software. Data are expressed as the mean±SEM pMOB1 levels relative to the vehicle control (n=3); ns>0.033, * p≤0.033, ** p≤ 0.002, *** p≤0.001 by one-way ANOVA with Fisher's LSD multiple comparison test.

FIG. 3A displays the Western blot of p-MOB1. MOB1. STK4, and GAPDH levels in mouse liver at 1, 2, or 3 h after intraperitoneal injection of vehicle or compound 11 at 10 mg/kg. Blots are representative of biological triplicates.

FIG. 3B shows densitometric quantification of p-MOB1 level relative to the vehicle control. Mean±SEM, n=3.

FIG. 3C shows LC-MS/MS quantification of compound 11 in mouse liver at 1, 2, or 3 h after intraperitoneal injection of vehicle or compound 11 at 10 mg/kg. Mean±SEM, n=3. Symbols represent individual mice.

FIG. 4A displays the Western blot of STK4, and GAPDH in MOLM 13 and MV4:11 cells transfected with doxycycline (dox)-inducible control (shScr) or STK4-specific shRNAs. Cells were analyzed 24 h after induction.

FIG. 4B displays the Western blot of STK3, and GAPDH in MOLM 13 and MV4:11 cells transfected with doxycycline (dox)-inducible control (shScr) or STK3-specific shRNAs. Cells were analyzed 24 h after induction.

FIG. 4C shows the proliferation assay of MOLM 13 cells stably expressing dox-inducible control or STK4-specific shRNAs. Cell viability was measured on day 0, 2, 4, 6, 8 (RLU) using the CellTiter-Glo assay (Promega). For every group, the average RLU value for day 0 was used to calculate fold change. To determine the relative decrease in cell proliferation for shSTK4, the average fold change for the shScr was subtracted from both groups for each day. ** p<0.002, *** p<0.001 by two-way ANOVA with Fisher's LSD multiple comparison test. Results represent three independent experiments performed in quadruplicate.

FIG. 4D shows the proliferation assay of MV4:11 cells stably expressing dox-inducible control or STK4-specific shRNAs. Cell viability was measured on day 0, 2, 4, 6, 8 (RLU) using the CellTiter-Glo assay (Promega). For every group, the average RLU value for day 0 was used to calculate fold change. To determine the relative decrease in cell proliferation for shSTK4, the average fold change for the shScr was subtracted from both groups for each day. ** p<0.002, *** p<0.001 by two-way ANOVA with Fisher's LSD multiple comparison test. Results represent three independent experiments performed in quadruplicate.

FIG. 4E shows the proliferation assay of MOLM 13 cells stably expressing dox-inducible control or STK3-specific shRNAs. Cell viability was measured on day 0, 2, 4, 6, 8 (RLU) using the CellTiter-Glo assay (Promega). For every group, the average RLU value for day 0 was used to calculate fold change. To determine the relative decrease in cell proliferation for shSTK3, the average fold change for the shScr was subtracted from both groups for each day. ** p<0.002, *** p<0.001 by two-way ANOVA with Fisher's LSD multiple comparison test. Results represent three independent experiments performed in quadruplicate.

FIG. 4F shows the proliferation assay of MV4:11 cells stably expressing dox-inducible control or STK3-specific shRNAs. Cell viability was measured on day 0, 2, 4, 6, 8 (RLU) using the CellTiter-Glo assay (Promega). For every group, the average RLU value for day 0 was used to calculate fold change. To determine the relative decrease in cell proliferation for shSTK3, the average fold change for the shScr was subtracted from both groups for each day. ** p<0.002, *** p<0.001 by two-way ANOVA with Fisher's LSD multiple comparison test. Results represent three independent experiments performed in quadruplicate.

### DETAILED DESCRIPTION

Serine/threonine-protein kinases 3 (STK3/MST2) and 4 (STK4/MST1) are the principal upstream kinases of the Hippo signaling pathway, which regulates cell proliferation, differentiation, and apoptosis. STK3/4 phosphorylate the large tumor suppressor kinases 1 and 2 (LATS1/LATS2) and the Mob kinase activators 1A and 1B (MOB1A/B). Activated LATS1/2 in association with MOB1A/B phosphorylate the major Hippo pathway downstream effectors. Yes-associated protein 1 (YAP) and transcriptional coactivator with PDZ-binding motif (TAZ) to negatively regulate their activity. Active YAP/TAZ translocate into the nucleus and interact mainly with the transcriptional enhanced associate domain (TEAD) transcription factors. The YAP/TAZ-TEAD protein complex controls the transcription of genes that regulate cell proliferation, apoptosis, and cell fate. Despite growing evidence to support a role for STK3/4 in various pathologies, the function of these kinases in tumorigenesis is unclear and appears to be context dependent. For example, STK3/4 suppress tumorigenesis in liver and lung cancer, whereas aberrant STK3/4 expression is associated with the progression of prostate and pancreatic cancer and the survival of glioblastoma cells.

Acute myeloid leukemia (AML) is an umbrella term for a diverse collection of hematological cancers characterized by excessive production of immature myeloid cells in the bone marrow. Although the various AML subtypes have differing prognoses, more than 30% of AML patients fail to enter complete remission after standard chemotherapy regimens. Thus, there is a significant unmet need for novel therapeutic targets and drugs to treat this disease. Recently, a large-scale loss-of-function RNAi screen of patient-derived AML cells identified STK3 as a potential target for antileukemia therapy. Indeed. STK3 depletion induces apoptotic cell death in some human AML cell lines and primary cells, and genetic inactivation of STK4 in multiple myeloma cells decreases their proliferation and induces a robust apoptotic response through YAP1 both in vitro and in vivo. Together, these data indicate that STK3/4 may present antileukemia targets.

The STK3/4 kinases have been shown to directly regulate the autophagy pathway. Initial large-scale proteomic analysis by Behrends et al., revealed that STK3 and STK4 are human ATG8 (LC3) interacting proteins. Notably. STK3/STK4 phosphorylate LC3 at the threonine 50 site. This phosphorylation is an essential step in autophagy, as a loss of phosphorylation at this site was shown to block the autophagy process. The Thr50 p-LC3 decreases the interaction between LC3 and FYVE And Coiled-Coil Domain Autophagy Adaptor 1 (FYCO1). STK3/4 are proposed to modulate autophagy by regulating the subcellular localization of autophagosomes.

From the standpoint of useful chemical probes only one STK3/4 inhibitor, XMU-MP-1, has been reported, however, this compound has some notable off-target activity in vitro, including inhibition of ULK1/2 and Aurora kinases. There remains a need to elucidate potent and selective small molecule inhibitor of STK3/4.

### Compounds

Disclosed herein is a compound of Formula (I), or a pharmaceutically acceptable salt thereof:

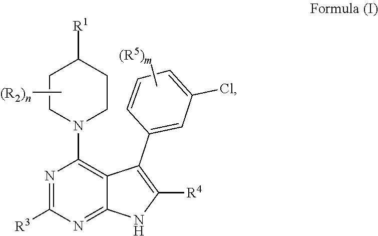

wherein:
R¹ is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{1a};
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{1a} on the same atom are taken together to form an oxo;
each R₂ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
n is 0-6;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R⁹ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (I), R³ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (I), R³ is hydrogen.

In some embodiments of a compound of Formula (I), R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (I), R⁴ is hydrogen.

In some embodiments of a compound of Formula (I), each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (I), each R⁵ is independently halogen, —CN, —OH, —OR^{a}, —NR^{c}R^{d}, C₁-C₆alkyl, or C₁-C₆haloalkyl. In some embodiments of a compound of Formula (I), each R⁵ is independently halogen, C₁-C₆alkyl, or C₁-C₆haloalkyl. In some embodiments of a compound of Formula (I), each R⁵ is independently halogen or C₁-C₆alkyl.

In some embodiments of a compound of Formula (I), m is 0 or 1. In some embodiments of a compound of Formula (I), m is 0-2. In some embodiments of a compound of Formula (I), m is 0-3. In some embodiments of a compound of Formula (I), m is 1 or 2. In some embodiments of a compound of Formula (I), m is 0. In some embodiments of a compound of Formula (I), m is 1. In some embodiments of a compound of Formula (I), m is 2. In some embodiments of a compound of Formula (I), m is 3.

In some embodiments of a compound of Formula (I), each R₂ is independently halogen, C₁-C₆alkyl, or C₁-C₆haloalkyl.

In some embodiments of a compound of Formula (I), n is 0 or 1. In some embodiments of a compound of Formula (I), n is 0. In some embodiments of a compound of Formula (I), n is 1. In some embodiments of a compound of Formula (I), n is 2. In some embodiments of a compound of Formula (I), n is 3.

In some embodiments of a compound of Formula (I), R¹ is halogen, —CN, —OH, —OR^{a}, —OC(═O)R^{a}, —OC(═O)OR^{b}, —OC(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl is optionally and independently substituted with one or more R^{1a}. In some embodiments of a compound of Formula (I), R¹ is-OH, —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{1a}. In some embodiments of a compound of Formula (I), R¹ is-OH, —OR^{a}, —NR^{c}R^{d}, or —NR^{b}C(═O)R^{a}.

In some embodiments of a compound of Formula (I), the compound is

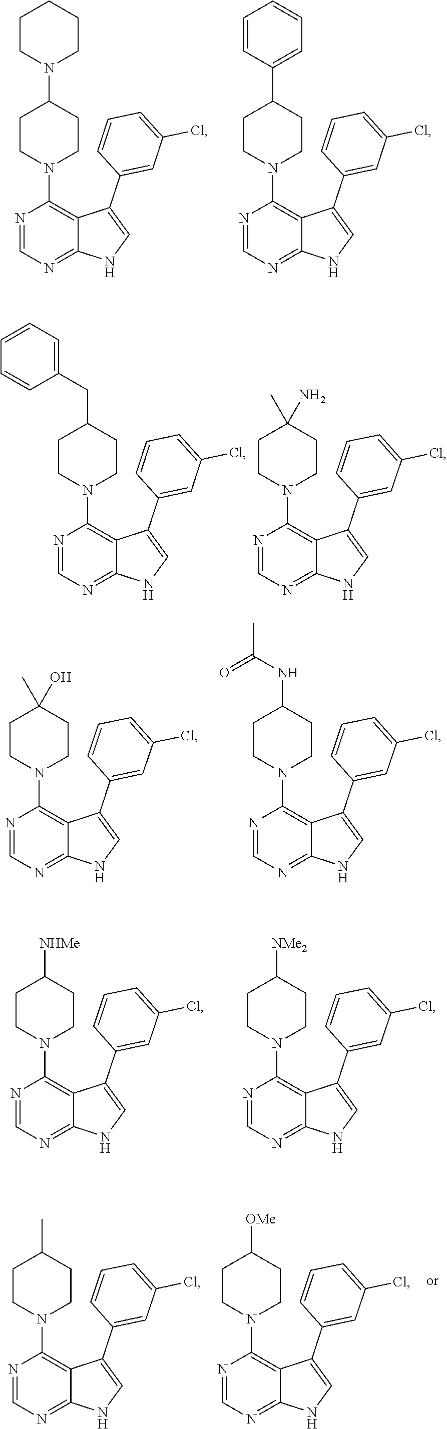

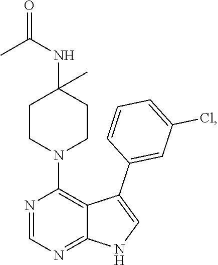 [SMILES: CC(=O)NC1(C)CCN(c2ncnc3[nH]cc(-c4cccc(Cl)c4)c23)CC1]

or a pharmaceutically acceptable salt thereof.

Also disclosed herein is a compound of Formula (II), or a pharmaceutically acceptable salt thereof:

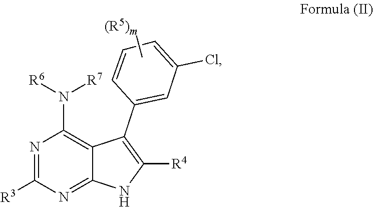

wherein:
R⁶ is hydrogen, C₁-C₆alkyl, C₁-C₆haloalkyl, C₁-C₆hydroxyalkyl, C₁-C₆aminoalkyl, or C₁-C₆heteroalkyl;
R⁷ is C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl; wherein the alkyl, alkenyl, and alkynyl is optionally and independently substituted with one or more R^{7a};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{7b};
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{7b} on the same atom are taken together to form an oxo;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl:
   provided that the compound is not:

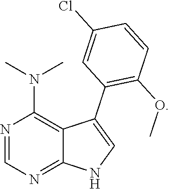 [SMILES: COc1ccc(Cl)cc1-c1c[nH]c2ncnc(N(C)C)c12]

In some embodiments of a compound of Formula (II), R³ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (II), R³ is hydrogen.

In some embodiments of a compound of Formula (II), R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (II), R⁴ is hydrogen.

In some embodiments of a compound of Formula (II), each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (II), each R⁵ is independently halogen, —CN, —OH, —OR^{a}, —NR^{c}R^{d}, C₁-C₆alkyl, or C₁-C₆haloalkyl. In some embodiments of a compound of Formula (II), each R⁵ is independently halogen, C₁-C₆alkyl, or C₁-C₆haloalkyl. In some embodiments of a compound of Formula (II), each R⁵ is independently halogen or C₁-C₆alkyl.

In some embodiments of a compound of Formula (II), m is 0 or 1. In some embodiments of a compound of Formula (II), m is 0-2. In some embodiments of a compound of Formula (II), m is 0 3. In some embodiments of a compound of Formula (II), m is 1 or 2. In some embodiments of a compound of Formula (II), m is 0. In some embodiments of a compound of Formula (II), m is 1. In some embodiments of a compound of Formula (II), m is 2. In some embodiments of a compound of Formula (II), m is 3.

In some embodiments of a compound of Formula (II). R⁶ is hydrogen or C₁-C₆alkyl. In some embodiments of a compound of Formula (II). R⁶ is hydrogen.

In some embodiments of a compound of Formula (II). R⁷ is C₁-C₆alkyl, C₁-C₆haloalkyl. C₁-C₆hydroxyalkyl, C₁-C₆aminoalkyl, C₁-C₆heteroalkyl, C₂-C₆alkenyl, or C₂-C₆alkynyl. In some embodiments of a compound of Formula (II). R⁷ is C₂-C₆alkyl, C₁-C₆haloalkyl, C₁-C₆hydroxyalkyl. C₁-C₆aminoalkyl, C₁-C₆heteroalkyl, C₂-C₆alkenyl, or C₂-C₆alkynyl. The compound of any one of claims 17-28, or a pharmaceutically acceptable salt thereof, wherein R⁷ is C₁-C₆alkyl, C₁-C₆haloalkyl. C₁-C₆hydroxyalkyl, or C₁-C₆aminoalkyl. In some embodiments of a compound of Formula (II). R⁷ is C₁-C₆alkyl, C₁-C₆hydroxyalkyl, or C₁-C₆aminoalkyl. In some embodiments of a compound of Formula (II). R⁷ is C₂-C₆alkyl, C₁-C₆hydroxyalkyl, or C₁-C₆aminoalkyl. In some embodiments of a compound of Formula (II). R⁷ is C₁-C₆alkyl optionally and independently substituted with one or more R^{7a}.

In some embodiments of a compound of Formula (II), each R^{7a} is independently halogen, —CN, —OH, —OR^{a}, —NR^{c}R^{d}, —NR^{b}C(═O)NR^{c}R^{d}, —NR^{b}C(═O)R^{a}, —C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{7b}. In some embodiments of a compound of Formula (II), each R^{7a} is independently halogen, —OH, —OR^{a}, —NR^{c}R^{d}, —NR^{b}C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{7b}. In some embodiments of a compound of Formula (II), each R^{7a} is independently halogen, —OH, —OR^{a}, —NR^{c}R^{d}, —NR^{b}C(═O)R^{a}, —C(═O)OR^{b}, —C(═O)NR^{c}R^{d}.

In some embodiments of a compound of Formula (II), each R^{7a} is independently cycloalkyl, heterocycloalkyl, aryl, or heteroaryl; wherein the cycloalkyl, heterocycloalkyl, aryl, and heteroaryl is optionally and independently substituted with one or more R^{7b}.

In some embodiments of a compound of Formula (II), each R^{7b} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (II), each R^{7b} is independently halogen, —OH, —OR^{a}, C₁-C₆alkyl, or C₁-C₆haloalkyl.

In some embodiments of a compound of Formula (II), the compound is

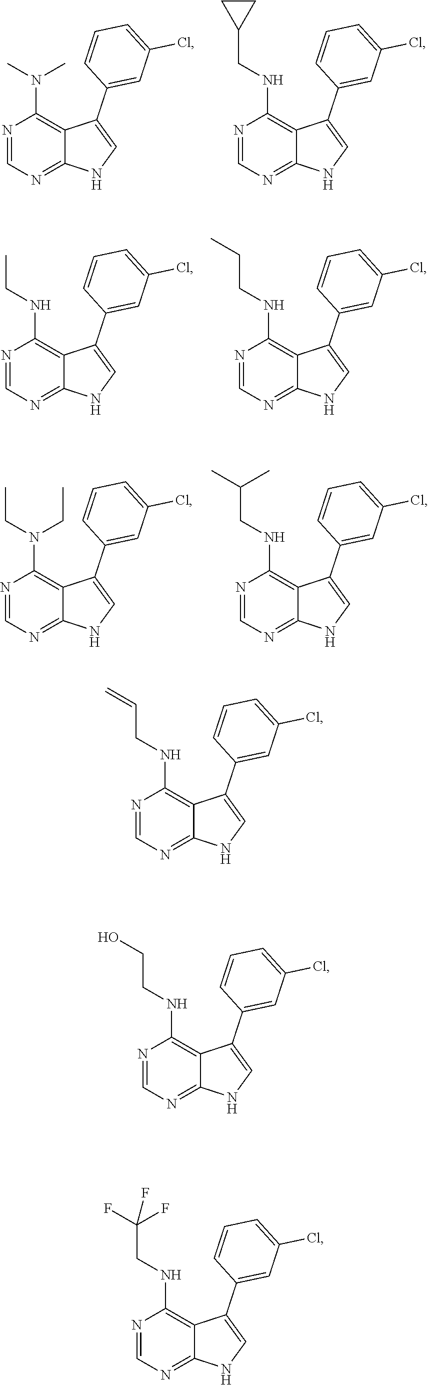

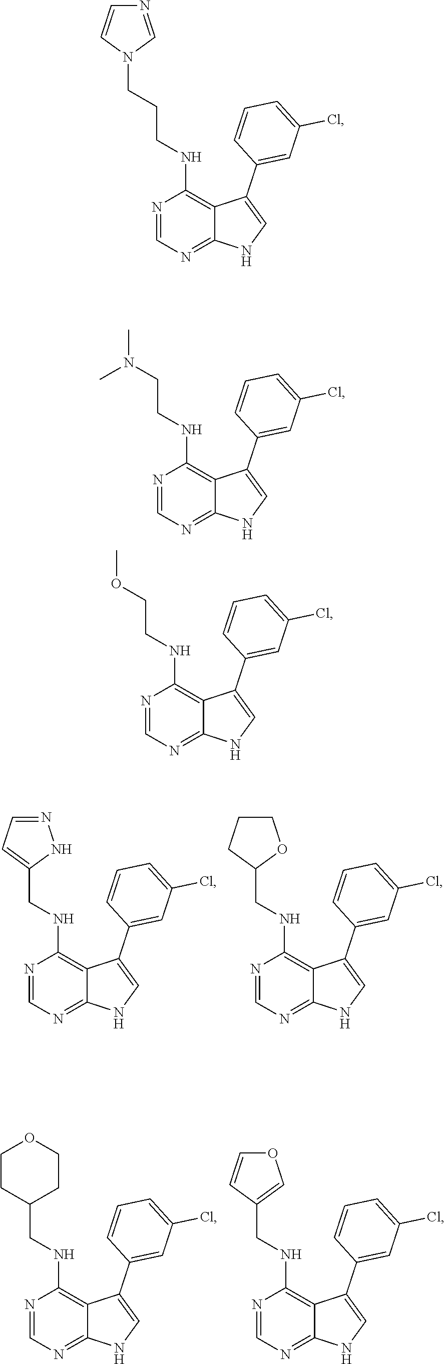

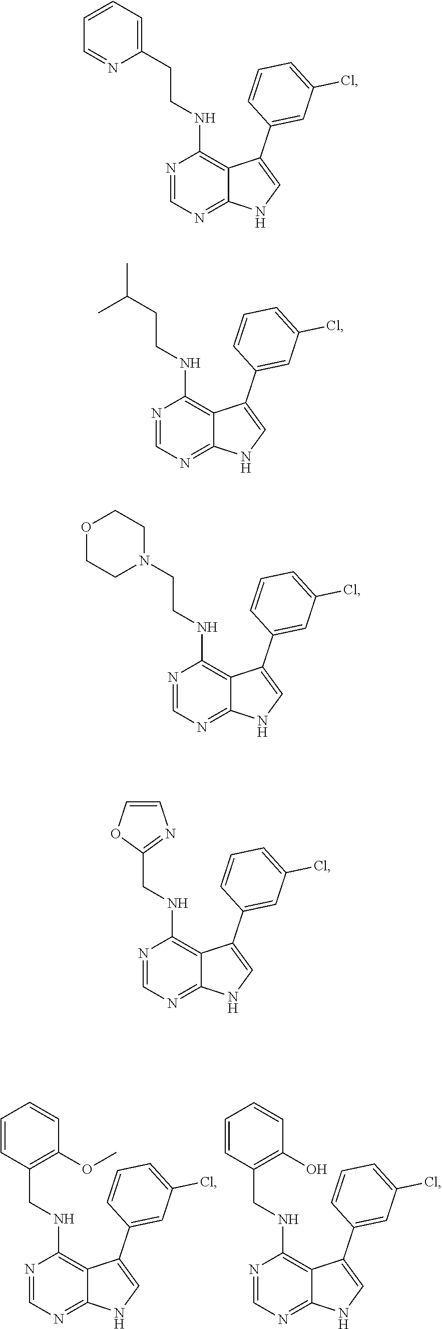

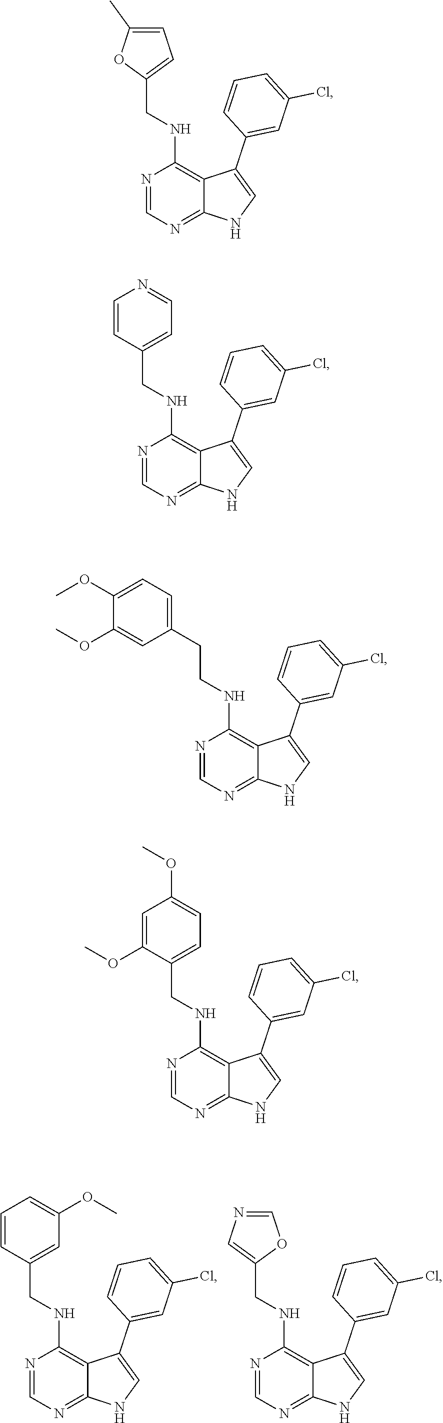

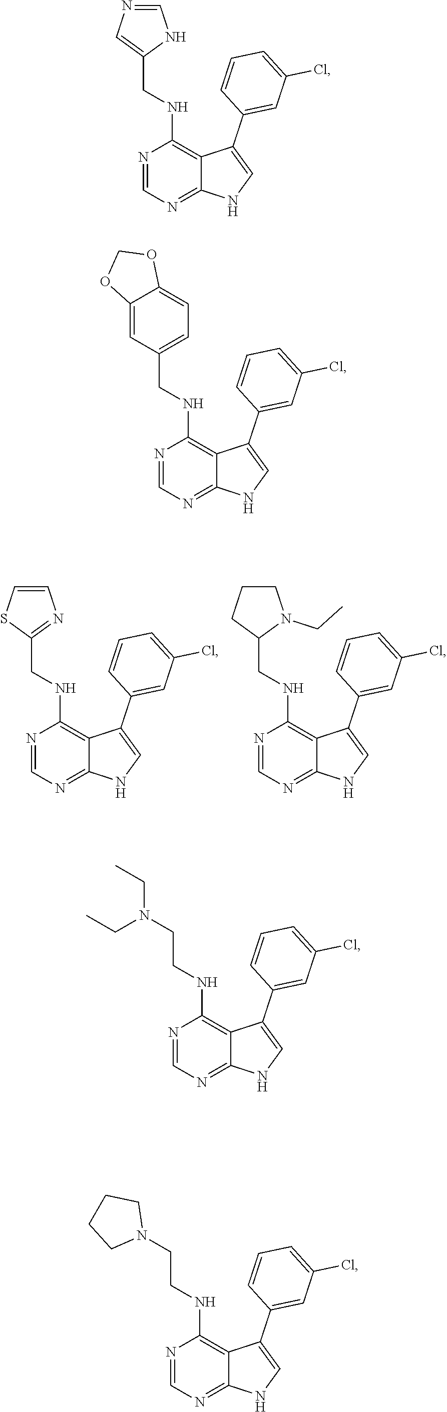

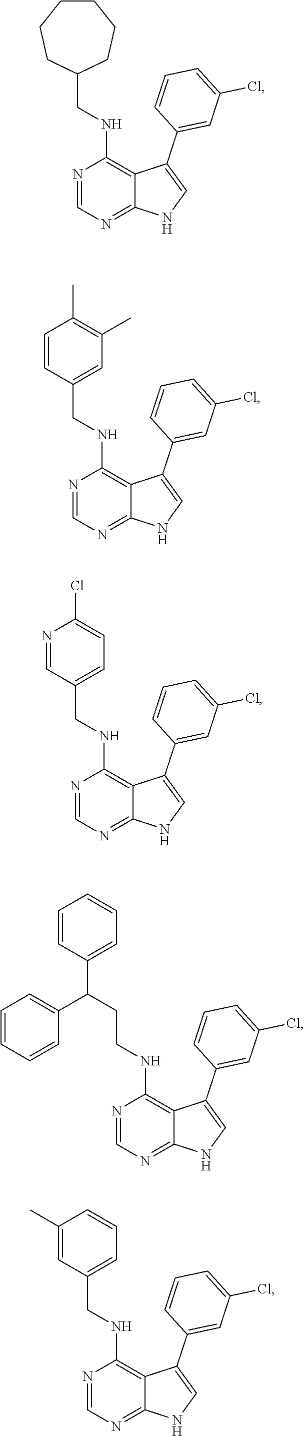 [SMILES: Clc1cccc(-c2c[nH]c3ncnc(NCC4CCCCCC4)c23)c1 ; Cc1ccc(CNc2ncnc3[nH]cc(-c4cccc(Cl)c4)c23)cc1C ; Clc1cccc(-c2c[nH]c3ncnc(NCc4ccc(Cl)nc4)c23)c1 ; Clc1cccc(-c2c[nH]c3ncnc(NCCC(c4ccccc4)c4ccccc4)c23)c1 ; Cc1cccc(CNc2ncnc3[nH]cc(-c4cccc(Cl)c4)c23)c1]

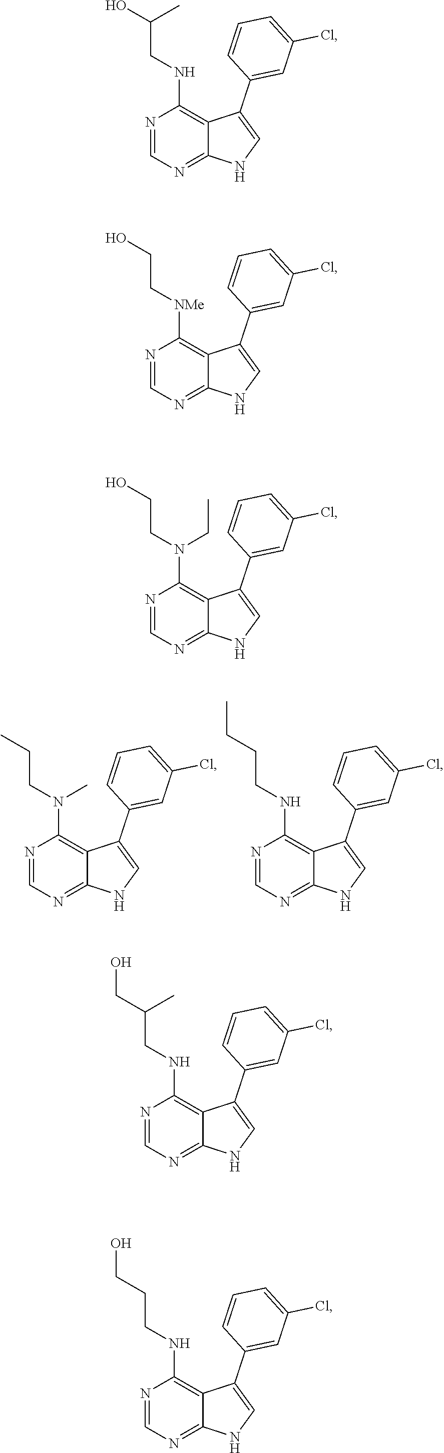

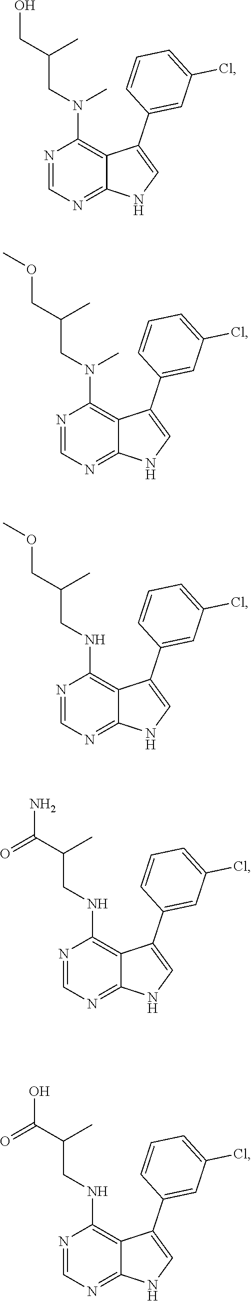 [SMILES: CC(CO)CN(C)c1ncnc2[nH]cc(-c3cccc(Cl)c3)c12 ; COCC(C)CN(C)c1ncnc2[nH]cc(-c3cccc(Cl)c3)c12 ; COCC(C)CNc1ncnc2[nH]cc(-c3cccc(Cl)c3)c12 ; CC(CNc1ncnc2[nH]cc(-c3cccc(Cl)c3)c12)C(N)=O ; CC(CNc1ncnc2[nH]cc(-c3cccc(Cl)c3)c12)C(=O)O]

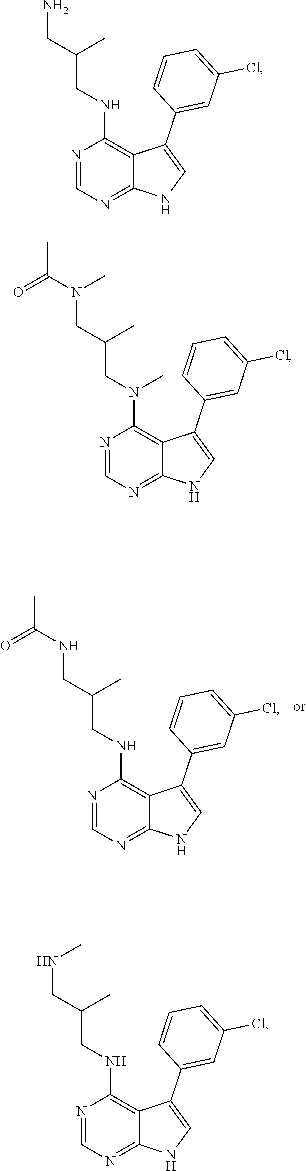 [SMILES: CC(CN)CNc1ncnc2[nH]cc(-c3cccc(Cl)c3)c12 ; CC(=O)N(C)CC(C)CN(C)c1ncnc2[nH]cc(-c3cccc(Cl)c3)c12 ; CC(=O)NCC(C)CNc1ncnc2[nH]cc(-c3cccc(Cl)c3)c12 ; CNCC(C)CNc1ncnc2[nH]cc(-c3cccc(Cl)c3)c12]

or a pharmaceutically acceptable salt thereof.

Also disclosed herein is a compound of Formula (III), or a pharmaceutically acceptable salt thereof:

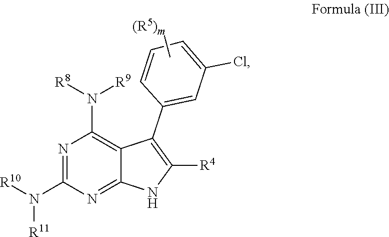

wherein:
R⁸ is hydrogen, C₁-C₆alkyl, C₁-C₆haloalkyl, C₁-C₆hydroxyalkyl, C₁-C₆aminoalkyl, or C₁-C₆heteroalkyl;
R⁹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the cycloalkyl, heterocycloalkyl, aryl, and heteroaryl is optionally and independently substituted with one or more R^{9b};
or two R^{9a} on the same atom are taken together to form an oxo;
each R^{9b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{9b} on the same atom are taken together to form an oxo;
or R⁸ and R⁹ are taken together with the atom to which they are attached to form a heterocycloalkyl optionally substituted with one or more R^{8a};
each R^{8a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{8a} on the same atom are taken together to form an oxo;
R¹⁰ is hydrogen, C₁-C₆alkyl, or C₁-C₆haloalkyl;
R¹¹ is C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{11a};
each R^{11a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{11a} on the same atom are taken together to form an oxo;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (III), R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (III), R⁴ is hydrogen.

In some embodiments of a compound of Formula (III), each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (III), each R⁵ is independently halogen, —CN, —OH, —OR^{a}, —NR^{c}R^{d}, C₁-C₆alkyl, or C₁-C₆haloalkyl. In some embodiments of a compound of Formula (III), each R⁵ is independently halogen, C₁-C₆alkyl, or C₁-C₆haloalkyl. In some embodiments of a compound of Formula (III), each R⁵ is independently halogen or C₁-C₆alkyl.

In some embodiments of a compound of Formula (III), m is 0 or 1. In some embodiments of a compound of Formula (III), m is 0-2. In some embodiments of a compound of Formula (III), m is 0-3. In some embodiments of a compound of Formula (III), m is 1 or 2. In some embodiments of a compound of Formula (III), m is 0. In some embodiments of a compound of Formula (III), m is 1. In some embodiments of a compound of Formula (III), m is 2. In some embodiments of a compound of Formula (III), m is 3.

In some embodiments of a compound of Formula (III), R¹⁰ is hydrogen or C₁-C₆alkyl. In some embodiments of a compound of Formula (III), R¹⁰ is hydrogen.

In some embodiments of a compound of Formula (III). R¹¹ is aryl optionally and independently substituted with one or more R^{11a}.

In some embodiments of a compound of Formula (III), each R^{11a} is independently halogen. —CN, —NO₂, —OH, —OR^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl. C₁-C₆haloalkyl, C₁-C₆hydroxyalkyl, C₁-C₆aminoalkyl, or C₁-C₆heteroalkyl. In some embodiments of a compound of Formula (III), each R^{11a} is independently halogen, —CN, —NO₂, —S(═O)₂NR^{c}R^{d}, or —C(═O)OR^{b}.

In some embodiments of a compound of Formula (III). R⁸ is hydrogen or C₁-C₆alkyl. In some embodiments of a compound of Formula (III). R⁸ is hydrogen.

In some embodiments of a compound of Formula (III). R⁹ is C₁-C₆alkyl, C₁-C₆haloalkyl. C₁-C₆hydroxyalkyl, C₁-C₆aminoalkyl, C₁-C₆heteroalkyl, C₂-C₆alkenyl, or C₂-C₆alkynyl. In some embodiments of a compound of Formula (III). R⁹ is C₁-C₆alkyl, C₁-C₆haloalkyl, C₁-C₆hydroxyalkyl, or C₁-C₆aminoalkyl. In some embodiments of a compound of Formula (III). R⁹ is C₁-C₆alkyl. C₁-C₆hydroxyalkyl, or C₁-C₆aminoalkyl. In some embodiments of a compound of Formula (III). R⁹ is C₁-C₆alkyl optionally and independently substituted with one or more R^{9a}.

In some embodiments of a compound of Formula (III), each R^{9a} is independently halogen, —CN, —OH, —OR^{a}, —NR^{c}R^{d}, —NR^{b}C(═O)NR^{c}R^{d}, —NR^{b}C(═O)R^{a}, —C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{9b}. In some embodiments of a compound of Formula (III), each R^{9a} is independently halogen, —OH, —OR^{a}, —NR^{c}R^{d}, —NR^{b}C(═O)R^{a}. —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{9b}. In some embodiments of a compound of Formula (III), each R^{9a} is independently halogen, —OH, —OR^{a}, —NR^{c}R^{d}, —NR^{b}C(═O)R^{a}, —C(═O)OR^{b}, —C(═O)NR^{c}R^{d}, In some embodiments of a compound of Formula (III), each R^{9a} is independently cycloalkyl, heterocycloalkyl, aryl, or heteroaryl; wherein the cycloalkyl, heterocycloalkyl, aryl, and heteroaryl is optionally and independently substituted with one or more R^{9b}.

In some embodiments of a compound of Formula (III), each R^{9b} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl. C₁-C₆hydroxyalkyl, C₁-C₆aminoalkyl, or C₁-C₆heteroalkyl. In some embodiments of a compound of Formula (III), each R^{9b} is independently halogen, —OH, —OR^{a}, C₁-C₆alkyl, or C₁-C₆haloalkyl.

In some embodiments of a compound of Formula (III). R⁸ and R⁹ are taken together to form a heterocycloalkyl optionally substituted with one or more R^{8a}. In some embodiments of a compound of Formula (III), the heterocycloalkyl formed when R⁸ and R⁹ are taken together is piperidine or morpholine.

In some embodiments of a compound of Formula (III), each R^{8a} is independently halogen, —CN, —OH, —OR^{a}, —OC(═O)R^{a}, —OC(═O)OR^{b}, —OC(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl. In some embodiments of a compound of Formula (III), R^{8a} is independently-OH, —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, cycloalkyl, heterocycloalkyl, aryl, or heteroaryl. In some embodiments of a compound of Formula (III), R^{8a} is independently-OH, —OR^{a}, —NR^{c}R^{d}, or —NR^{b}C(═O)R^{a}. In some embodiments of a compound of Formula (III), each R^{8a} is-OH.

In some embodiments of a compound of Formula (III), the compound is

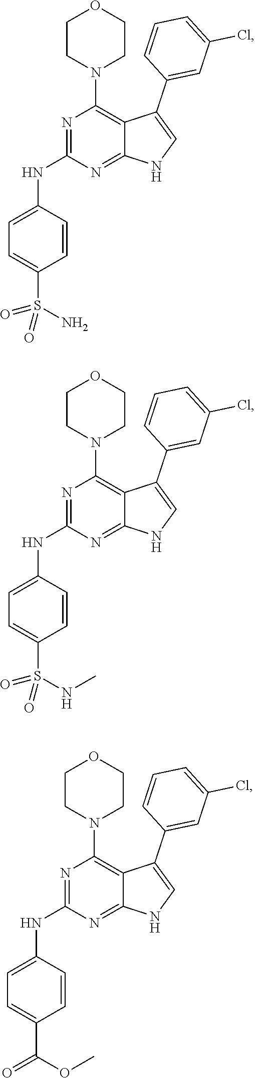 [SMILES: NS(=O)(=O)c1ccc(Nc2nc(N3CCOCC3)c3c(-c4cccc(Cl)c4)c[nH]c3n2)cc1 ; CNS(=O)(=O)c1ccc(Nc2nc(N3CCOCC3)c3c(-c4cccc(Cl)c4)c[nH]c3n2)cc1 ; COC(=O)c1ccc(Nc2nc(N3CCOCC3)c3c(-c4cccc(Cl)c4)c[nH]c3n2)cc1]

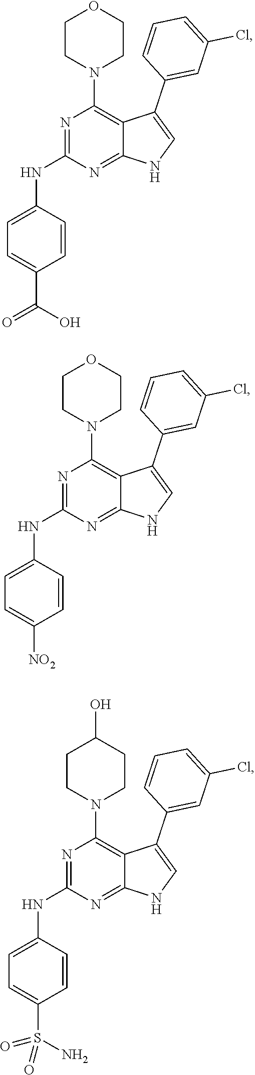

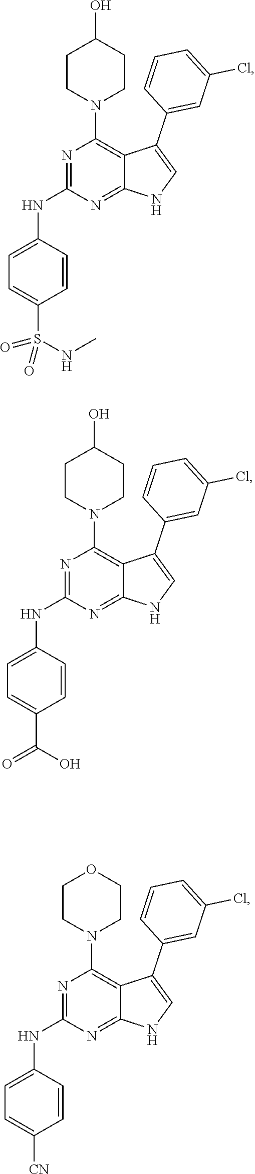 [SMILES: CNS(=O)(=O)c1ccc(Nc2nc(N3CCC(O)CC3)c3c(-c4cccc(Cl)c4)c[nH]c3n2)cc1 ; O=C(O)c1ccc(Nc2nc(N3CCC(O)CC3)c3c(-c4cccc(Cl)c4)c[nH]c3n2)cc1 ; N#Cc1ccc(Nc2nc(N3CCOCC3)c3c(-c4cccc(Cl)c4)c[nH]c3n2)cc1]

or a pharmaceutically acceptable salt thereof.

Also disclosed herein is a compound of Formula (A), or a pharmaceutically acceptable salt thereof:

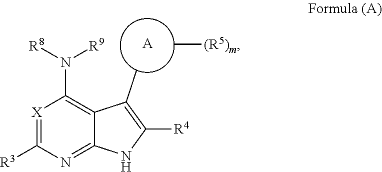

wherein:
X is N or CR^{X};
R^{X} is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
Ring A is aryl or heteroaryl;
each R³ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d} C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 0-4;
R⁸ is hydrogen, C₁-C₆alkyl, C₁-C₆haloalkyl, C₁-C₆hydroxyalkyl, C₁-C₆aminoalkyl, or C₁-C₆heteroalkyl;
R⁹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the cycloalkyl, heterocycloalkyl, aryl, and heteroaryl is optionally and independently substituted with one or more R^{9b};
or two R^{9a} on the same atom are taken together to form an oxo;
each R^{9b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
or two R^{9b} on the same atom are taken together to form an oxo;
or R⁸ and R⁹ are taken together with the atom to which they are attached to form a heterocycloalkyl optionally substituted with one or more R^{8a};
each R^{8a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O); R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
or two R^{8a} on the same atom are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
each R⁹ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
   provided that the compound is not

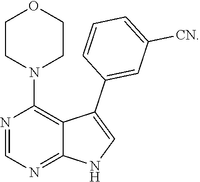 [SMILES: N#Cc1cccc(-c2c[nH]c3ncnc(N4CCOCC4)c23)c1]

In some embodiments of a compound of Formula (A), X is N. In some embodiments of a compound of Formula (A), X is CR^{X}.

In some embodiments of a compound of Formula (A), Rx is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (A), R^{X} is hydrogen.

In some embodiments of a compound of Formula (A), R³ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (A), R³ is hydrogen.

In some embodiments of a compound of Formula (A), R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (A), R⁴ is hydrogen.

In some embodiments of a compound of Formula (A), Ring A is phenyl. In some embodiments of a compound of Formula (A), Ring A is 5- or 6-membered heteroaryl.

In some embodiments of a compound of Formula (A), each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (A), each R⁵ is independently halogen, —CN, —OH, —OR^{a}, —NR^{c}R^{d}, C₁-C₆alkyl, or C₁-C₆haloalkyl. In some embodiments of a compound of Formula (A), each R⁵ is independently halogen, C₁-C₆alkyl, or C₁-C₆haloalkyl. In some embodiments of a compound of Formula (A), each R⁵ is independently halogen or C₁-C₆alkyl. In some embodiments of a compound of Formula (A). R⁵ is not-CN. In some embodiments of a compound of Formula (A), each R⁵ is independently halogen.

In some embodiments of a compound of Formula (A), m is 0 or 1. In some embodiments of a compound of Formula (A), m is 0-2. In some embodiments of a compound of Formula (A), m is 0-3. In some embodiments of a compound of Formula (A), m is 1 or 2. In some embodiments of a compound of Formula (A), m is 0. In some embodiments of a compound of Formula (III), m is 1. In some embodiments of a compound of Formula (A), m is 2. In some embodiments of a compound of Formula (A), m is 3.

In some embodiments of a compound of Formula (A). R⁸ is hydrogen or C₁-C₆alkyl. In some embodiments of a compound of Formula (A). R⁸ is hydrogen.

In some embodiments of a compound of Formula (A). R⁹ is C₁-C₆alkyl, C₁-C₆haloalkyl. C₁-C₆hydroxyalkyl, C₁-C₆aminoalkyl, C₁-C₆heteroalkyl, C₂-C₆alkenyl, or C₂-C₆alkynyl. In some embodiments of a compound of Formula (A). R⁹ is C₁-C₆alkyl, C₁-C₆haloalkyl, C₁-C₆hydroxyalkyl, or C₁-C₆aminoalkyl. In some embodiments of a compound of Formula (A). R⁹ is C₁-C₆alkyl. C₁-C₆hydroxyalkyl, or C₁-C₆aminoalkyl. In some embodiments of a compound of Formula (A). R⁹ is C₁-C₆alkyl optionally and independently substituted with one or more R^{9a}.

In some embodiments of a compound of Formula (A), each R^{9a} is independently halogen, —CN, —OH, —OR^{a}, —NR^{c}R^{d}, —NR^{b}C(═O)NR^{c}R^{d}, —NR^{b}C(═O)R^{a}, —C(═O)R^{a}—C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{9b}. In some embodiments of a compound of Formula (A), each R^{9a} is independently halogen, —OH, —OR^{a}, —NR^{c}R^{d}, —NR^{b}C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{9b}. In some embodiments of a compound of Formula (A), each R^{9a} is independently halogen, —OH, —OR^{a}, —NR^{c}R^{d}, —NR^{b}C(═O)R^{a}, —C(═O)OR^{b}, —C(═O)NR^{c}R^{d}, In some embodiments of a compound of Formula (A), each R^{9a} is independently cycloalkyl, heterocycloalkyl, aryl, or heteroaryl; wherein the cycloalkyl, heterocycloalkyl, aryl, and heteroaryl is optionally and independently substituted with one or more R^{9b}.

In some embodiments of a compound of Formula (A), each R^{9b} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl. C₁-C₆hydroxyalkyl, C₁-C₆aminoalkyl, or C₁-C₆heteroalkyl. In some embodiments of a compound of Formula (A), each R^{9b} is independently halogen, —OH, —OR^{a}, C₁-C₆alkyl, or C₁-C₆haloalkyl.

In some embodiments of a compound of Formula (A). R⁸ and R⁹ are taken together to form a heterocycloalkyl optionally substituted with one or more R^{8a}. In some embodiments of a compound of Formula (A), the heterocycloalkyl formed when R⁸ and R⁹ are taken together is piperidine or morpholine.

In some embodiments of a compound of Formula (A), each R^{8a} is independently halogen, —CN, —OH, —OR^{a}, —OC(═O)R^{a}, —OC(═O)OR^{b}, —OC(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl. C₁-C₆hydroxyalkyl, C₁-C₆aminoalkyl, C₁-C₆heteroalkyl, cycloalkyl, heterocycloalkyl, aryl, or heteroaryl.

In some embodiments of a compound of Formula (A), each R^{8a} is independently-OH, —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, cycloalkyl, heterocycloalkyl, aryl, or heteroaryl. In some embodiments of a compound of Formula (A), each R^{8a} is independently-OH, —OR^{a}, —NR^{c}R^{d}, or —NR^{b}C(═O)R^{a}. In some embodiments of a compound of Formula (A), each R^{8a} is-OH.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl. C₁-C₆haloalkyl, C₁-C₆hydroxyalkyl, C₁-C₆aminoalkyl, or C₁-C₆heteroalkyl. In some embodiments of a compound disclosed herein, each R^{a} is independently C₁-C₆alkyl, C₁-C₆haloalkyl. C₁-C₆hydroxyalkyl, C₁-C₆aminoalkyl, C₁-C₆heteroalkyl, cycloalkyl, or heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, heterocycloalkyl, aryl, and heteroaryl is independently optionally substituted with one or more oxo, halogen, —CN, —OH, —OC₁-C₆alkyl, —C₁-C₆haloalkyl, C₁-C₆hydroxyalkyl, C₁-C₆aminoalkyl, or C₁-C₆heteroalkyl. In some embodiments of a compound disclosed herein, each R^{b} is independently hydrogen, C₁-C₆alkyl, C₁-C₆haloalkyl. C₁-C₆hydroxyalkyl, C₁-C₆aminoalkyl, C₁-C₆heteroalkyl, cycloalkyl, or heterocycloalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each Rb is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl. C₁-C₆haloalkyl, C₁-C₆hydroxyalkyl, C₁-C₆aminoalkyl, or C₁-C₆heteroalkyl. In some embodiments of a compound disclosed herein, each R^{c} and R^{d} are independently hydrogen, C₁-C₆alkyl. C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein. R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl. C₁-C₆aminoalkyl, or C₁-C₆heteroalkyl. In some embodiments of a compound disclosed herein. R^{c} and R^{d} are taken together with the atom to which they are attached to form a heterocycloalkyl.

Any combination of the groups described above for the various variables is contemplated herein. Throughout the specification, groups and substituents thereof are chosen by one skilled in the field to provide stable moieties and compounds.

In some embodiments, the compound has the structure of any of the compounds in Table 1, or a pharmaceutically acceptable salt or solvate thereof.

TABLE 1

| Cmpd | | |
|---|---|---|
| No. | Name | Structure |
| 1 | 4-(5-Phenyl-7H-pyrrolo[2,3-d]pyrimidin- 4-yl)morpholine | 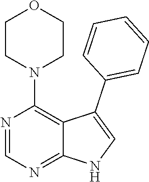 [SMILES: c1ccc(-c2c[nH]c3ncnc(N4CCOCC4)c23)cc1] |
| 2 | 4-(5-(o-Tolyl)-7H-pyrrolo[2,3- d]pyrimidin-4-yl)morpholine | 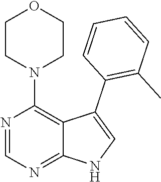 [SMILES: Cc1ccccc1-c1c[nH]c2ncnc(N3CCOCC3)c12] |
| 3 | 4-(5-(2-Fluorophenyl)-7H-pyrrolo[2,3- d]pyrimidin-4-yl)morpholine | 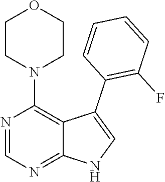 [SMILES: Fc1ccccc1-c1c[nH]c2ncnc(N3CCOCC3)c12] |
| 4 | 4-(5-(2-Methoxyphenyl)-7H-pyrrolo[2,3- d]pyrimidin-4-yl)morpholine | 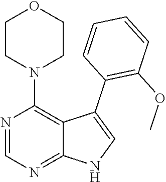 [SMILES: COc1ccccc1-c1c[nH]c2ncnc(N3CCOCC3)c12] |
| 5 | 4-(5-(m-Tolyl)-7H-pyrrolo[2,3- d]pyrimidin-4-yl)morpholine | 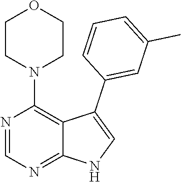 [SMILES: Cc1cccc(-c2c[nH]c3ncnc(N4CCOCC4)c23)c1] |
| 6 | 4-(5-(3-Chlorophenyl)-7H-pyrrolo[2,3- d]pyrimidin-4-yl)morpholine | 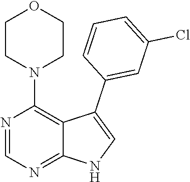 [SMILES: Clc1cccc(-c2c[nH]c3ncnc(N4CCOCC4)c23)c1] |
| 7 | 4-(5-(3-Fluorophenyl)-7H-pyrrolo[2,3- d]pyrimidin-4-yl)morpholine | 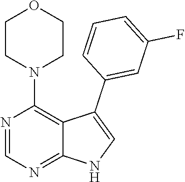 [SMILES: Fc1cccc(-c2c[nH]c3ncnc(N4CCOCC4)c23)c1] |
| 8 | 4-(5-(3-Methoxyphenyl)-7H-pyrrolo[2,3- d]pyrimidin-4-yl)morpholine | 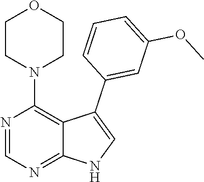 [SMILES: COc1cccc(-c2c[nH]c3ncnc(N4CCOCC4)c23)c1] |
| 9 | 4-(5-(p-tolyl)-7H-pyrrolo[2,3- d]pyrimidin-4-yl)morpholine | 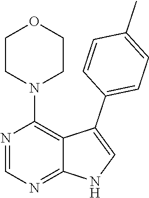 [SMILES: Cc1ccc(-c2c[nH]c3ncnc(N4CCOCC4)c23)cc1] |
| 10 | 4-(5-(4-Fluorophenyl)-7H-pyrrolo[2,3- d]pyrimidin-4-yl)morpholine | 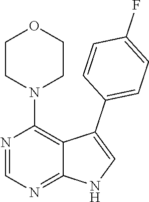 [SMILES: Fc1ccc(-c2c[nH]c3ncnc(N4CCOCC4)c23)cc1] |
| 11 | 3-(4-Morpholino-7H-pyrrolo[2,3- d]pyrimidin-5-yl)benzamide | 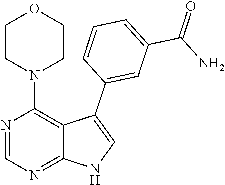 [SMILES: NC(=O)c1cccc(-c2c[nH]c3ncnc(N4CCOCC4)c23)c1] |
| 12 | 4-(5-(Thiophen-2-yl)-7H-pyrrolo[2,3- d]pyrimidin-4-yl)morpholine | 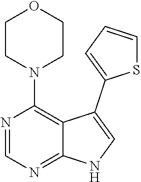 [SMILES: c1csc(-c2c[nH]c3ncnc(N4CCOCC4)c23)c1] |
| 13 | 4-(5-(Furan-2-yl)-7H-pyrrolo[2,3- d]pyrimidin-4-yl)morpholine | 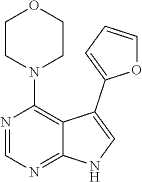 [SMILES: c1coc(-c2c[nH]c3ncnc(N4CCOCC4)c23)c1] |
| 14 | 4-(5-(Furan-3-yl)-7H-pyrrolo[2,3- d]pyrimidin-4-yl)morpholine | 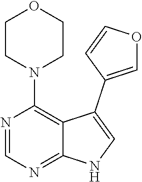 [SMILES: c1nc(N2CCOCC2)c2c(-c3ccoc3)c[nH]c2n1] |
| 15 | 5-(3-Chlorophenyl)-4-(piperidin-1-yl)- 7H-pyrrolo[2,3-d]pyrimidine | 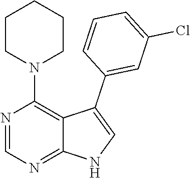 [SMILES: Clc1cccc(-c2c[nH]c3ncnc(N4CCCCC4)c23)c1] |
| 16 | 1-(5-(3-Chlorophenyl)-7H-pyrrolo[2,3- d]pyrimidin-4-yl)piperidin-4-ol | 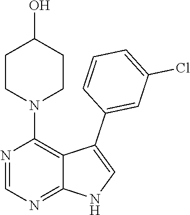 [SMILES: OC1CCN(c2ncnc3[nH]cc(-c4cccc(Cl)c4)c23)CC1] |
| 17 | 5-(3-Chlorophenyl)-N,N-dimethyl-7H- pyrrolo[2,3-d]pyrimidin-4-amine | 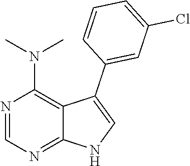 [SMILES: CN(C)c1ncnc2[nH]cc(-c3cccc(Cl)c3)c12] |
| 18 | 5-(3-Chlorophenyl)-N- (cyclopropylmethyl)-7H-pyrrolo[2,3- d]pyrimidin-4-amine | 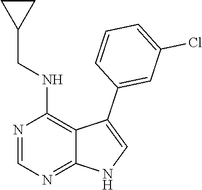 [SMILES: Clc1cccc(-c2c[nH]c3ncnc(NCC4CC4)c23)c1] |
| 19 | 4-(3-(3-chlorophenyl)-1H-pyrazolo[3,4- d]pyrimidin-4-yl)morpholine | 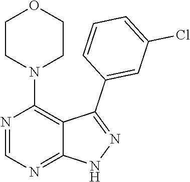 [SMILES: Clc1cccc(-c2n[nH]c3ncnc(N4CCOCC4)c23)c1] |
| 20 | 4-(5-(3-Chlorophenyl)thieno[2,3- d]pyrimidin-4-yl)morpholine | 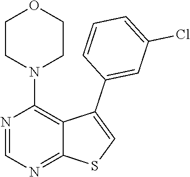 [SMILES: Clc1cccc(-c2csc3ncnc(N4CCOCC4)c23)c1] |
| 21 | 5-(3-Chlorophenyl)-4- morpholinofuro[2,3-d]pyrimidine | 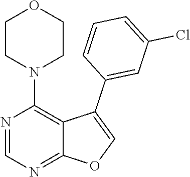 [SMILES: Clc1cccc(-c2coc3ncnc(N4CCOCC4)c23)c1] |
| 22 | 4-(3-(3-Chlorophenyl)-1H-pyrrolo[3,2- c]pyridin-4-yl)morpholin | 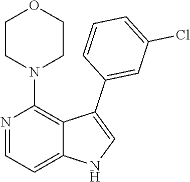 [SMILES: Clc1cccc(-c2c[nH]c3ccnc(N4CCOCC4)c23)c1] |
| 23 | 4-(3-(3-chlorophenyl)-1H-pyrrolo[2,3- b]pyridin-4-yl)morpholine | 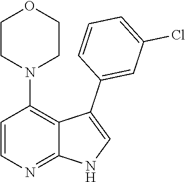 [SMILES: Clc1cccc(-c2c[nH]c3nccc(N4CCOCC4)c23)c1] |
| 24 | 4-(5-(2-Chlorophenyl)-7H-pyrrolo[2,3- d]pyrimidin-4-yl)morpholine | 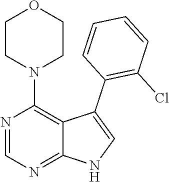 [SMILES: Clc1ccccc1-c1c[nH]c2ncnc(N3CCOCC3)c12] |
| 25 | 4-(5-(4-Chlorophenyl)-7H-pyrrolo[2,3- d]pyrimidin-4-yl)morpholine | 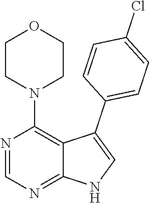 [SMILES: Clc1ccc(-c2c[nH]c3ncnc(N4CCOCC4)c23)cc1] |
| 26 | 4-(5-(4-methoxyphenyl)-7H-pyrrolo[2,3- d]pyrimidin-4-yl)morpholine | 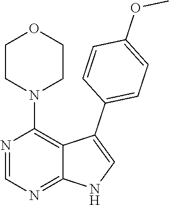 [SMILES: COc1ccc(-c2c[nH]c3ncnc(N4CCOCC4)c23)cc1] |
| 27 | 4-(5-(3-(trifluoromethoxy)phenyl)-7H- pyrrolo[2,3-d]pyrimidin-4-yl)morpholine | 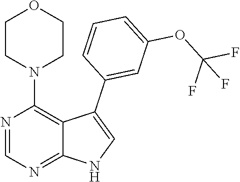 [SMILES: FC(F)(F)Oc1cccc(-c2c[nH]c3ncnc(N4CCOCC4)c23)c1] |
| 28 | 4-(5-(4-(trifluoromethoxy)phenyl)-7H- pyrrolo[2,3-d]pyrimidin-4-yl)morpholine | 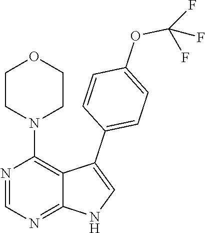 [SMILES: FC(F)(F)Oc1ccc(-c2c[nH]c3ncnc(N4CCOCC4)c23)cc1] |
| 29 | 1-(4-(4-morpholino-7H-pyrrolo[2,3- d]pyrimidin-5-yl)phenyl)ethan-1-one | 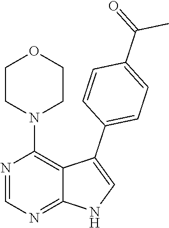 [SMILES: CC(=O)c1ccc(-c2c[nH]c3ncnc(N4CCOCC4)c23)cc1] |
| 30 | 4-(5-(3,4-dichlorophenyl)-7H- pyrrolo[2,3-d]pyrimidin-4-yl)morpholine | 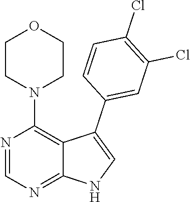 [SMILES: Clc1ccc(-c2c[nH]c3ncnc(N4CCOCC4)c23)cc1Cl] |
| 31 | 4-(5-(2-chloropyridin-4-yl)-7H- pyrrolo[2,3-d]pyrimidin-4-yl)morpholine | 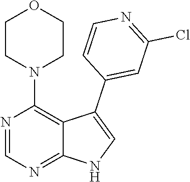 [SMILES: Clc1cc(-c2c[nH]c3ncnc(N4CCOCC4)c23)ccn1] |
| 32 | 4-(5-(2-chloro-5-fluoropyridin-4-yl)-7H- pyrrolo[2,3-d]pyrimidin-4-yl)morpholine | 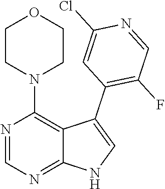 [SMILES: Fc1cnc(Cl)cc1-c1c[nH]c2ncnc(N3CCOCC3)c12] |
| 33 | 4-(5-(2-chloro-3-fluoropyridin-4-yl)-7H- pyrrolo[2,3-d]pyrimidin-4-yl)morpholine | 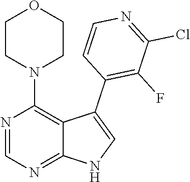 [SMILES: Fc1c(-c2c[nH]c3ncnc(N4CCOCC4)c23)ccnc1Cl] |
| 34 | 4-(5-(2-chloropyridin-3-yl)-7H- pyrrolo[2,3-d]pyrimidin-4-yl)morpholine | 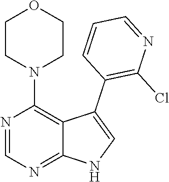 [SMILES: Clc1ncccc1-c1c[nH]c2ncnc(N3CCOCC3)c12] |
| 35 | 4-(5-(pyridin-4-yl)-7H-pyrrolo[2,3- d]pyrimidin-4-yl)morpholine | 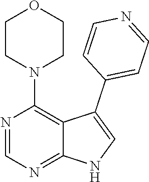 [SMILES: c1cc(-c2c[nH]c3ncnc(N4CCOCC4)c23)ccn1] |
| 36 | 4-(5-(pyrimidin-5-yl)-7H-pyrrolo[2,3- d]pyrimidin-4-yl)morpholine | 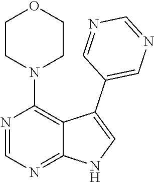 [SMILES: c1ncc(-c2c[nH]c3ncnc(N4CCOCC4)c23)cn1] |
| 37 | 4-(5-(3-methylpyridin-4-yl)-7H- pyrrolo[2,3-d]pyrimidin-4-yl)morpholine | 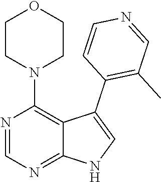 [SMILES: Cc1cnccc1-c1c[nH]c2ncnc(N3CCOCC3)c12] |
| 38 | 4-(5-(2-methoxypyrimidin-5-yl)-7H- pyrrolo[2,3-d]pyrimidin-4-yl)morpholine | 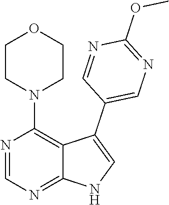 [SMILES: COc1ncc(-c2c[nH]c3ncnc(N4CCOCC4)c23)cn1] |
| 39 | 4-(5-(3-(methylsulfonyl)phenyl)-7H- pyrrolo[2,3-d]pyrimidin-4-yl)morpholine | 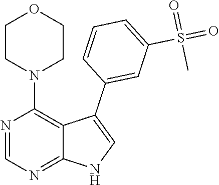 [SMILES: CS(=O)(=O)c1cccc(-c2c[nH]c3ncnc(N4CCOCC4)c23)c1] |
| 40 | 4-(5-(4-(methylsulfonyl)phenyl)-7H- pyrrolo[2,3-d]pyrimidin-4-yl)morpholine | 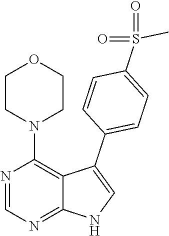 [SMILES: CS(=O)(=O)c1ccc(-c2c[nH]c3ncnc(N4CCOCC4)c23)cc1] |
| 41 | 4-(5-(1-methyl-1H-pyrazol-4-yl)-7H- pyrrolo[2,3-d]pyrimidin-4-yl)morpholine | 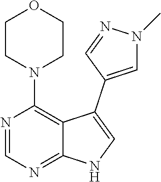 [SMILES: Cn1cc(-c2c[nH]c3ncnc(N4CCOCC4)c23)cn1] |
| 42 | 4-(5-(isoxazol-4-yl)-7H-pyrrolo[2,3- d]pyrimidin-4-yl)morpholine | 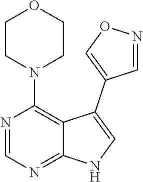 [SMILES: c1nc(N2CCOCC2)c2c(-c3cnoc3)c[nH]c2n1] |
| 43 | 4-(5-(3,5-dimethylisoxazol-4-yl)-7H- pyrrolo[2,3-d]pyrimidin-4-yl)morpholine | 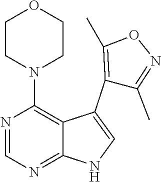 [SMILES: Cc1noc(C)c1-c1c[nH]c2ncnc(N3CCOCC3)c12] |
| 44 | 5-(3-chlorophenyl)-4-(pyrrolidin-1-yl)- 7H-pyrrolo[2,3-d]pyrimidine | 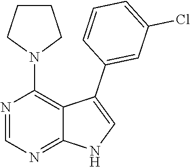 [SMILES: Clc1cccc(-c2c[nH]c3ncnc(N4CCCC4)c23)c1] |
| 45 | 1-(4-(5-(3-chlorophenyl)-7H-pyrrolo[2,3- d]pyrimidin-4-yl)piperazin-1-yl)ethan-1- one | 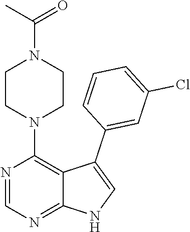 [SMILES: CC(=O)N1CCN(c2ncnc3[nH]cc(-c4cccc(Cl)c4)c23)CC1] |
| 46 | 4-(azetidin-1-yl)-5-(3-chlorophenyl)-7H- pyrrolo[2,3-d]pyrimidine | 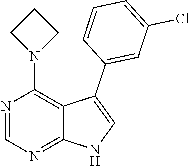 [SMILES: Clc1cccc(-c2c[nH]c3ncnc(N4CCC4)c23)c1] |
| 47 | 5-(3-chlorophenyl)-4-(4- methylpiperazin-1-yl)-7H-pyrrolo[2,3- d]pyrimidine | 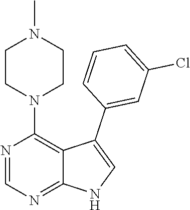 [SMILES: CN1CCN(c2ncnc3[nH]cc(-c4cccc(Cl)c4)c23)CC1] |
| 48 | 5-(3-chlorophenyl)-4-(4-ethylpiperazin- 1-yl)-7H-pyrrolo[2,3-d]pyrimidine | 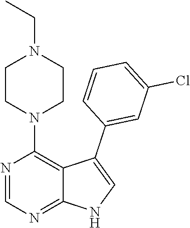 [SMILES: CCN1CCN(c2ncnc3[nH]cc(-c4cccc(Cl)c4)c23)CC1] |
| 49 | 5-(3-chlorophenyl)-N-ethyl-7H- pyrrolo[2,3-d]pyrimidin-4-amine | 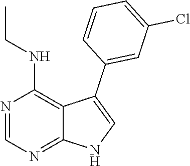 [SMILES: CCNc1ncnc2[nH]cc(-c3cccc(Cl)c3)c12] |
| 50 | 5-(3-chlorophenyl)-N-propyl-7H- pyrrolo[2,3-d]pyrimidin-4-amine | 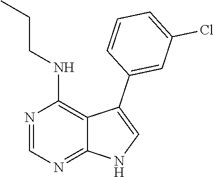 [SMILES: CCCNc1ncnc2[nH]cc(-c3cccc(Cl)c3)c12] |
| 51 | 5-(3-chlorophenyl)-N,N-diethyl-7H- pyrrolo[2,3-d]pyrimidin-4-amine | 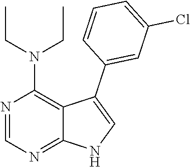 [SMILES: CCN(CC)c1ncnc2[nH]cc(-c3cccc(Cl)c3)c12] |
| 52 | 5-(3-chlorophenyl)-N-isobutyl-7H- pyrrolo[2,3-d]pyrimidin-4-amine | 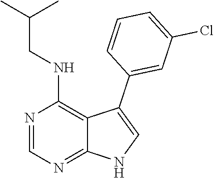 [SMILES: CC(C)CNc1ncnc2[nH]cc(-c3cccc(Cl)c3)c12] |
| 53 | N-allyl-5-(3-chlorophenyl)-7H- pyrrolo[2,3-d]pyrimidin-4-amine | 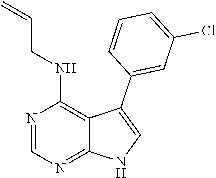 [SMILES: C=CCNc1ncnc2[nH]cc(-c3cccc(Cl)c3)c12] |
| 54 | 2-((5-(3-chlorophenyl)-7H-pyrrolo[2,3- d]pyrimidin-4-yl)amino)ethan-1-ol | 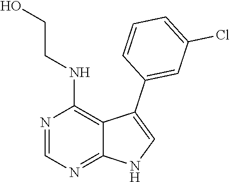 [SMILES: OCCNc1ncnc2[nH]cc(-c3cccc(Cl)c3)c12] |
| 55 | 5-(3-chlorophenyl)-N-(2,2,2- trifluoroethyl)-7H-pyrrolo[2,3- d]pyrimidin-4-amine | 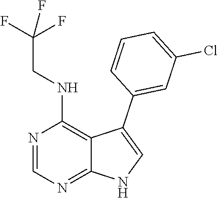 [SMILES: FC(F)(F)CNc1ncnc2[nH]cc(-c3cccc(Cl)c3)c12] |
| 56 | 5-(3-chlorophenyl)-4-(4-(cyclopenta-2,4- dien-1-yl)butyl)-7H-pyrrolo[2,3- d]pyrimidine | 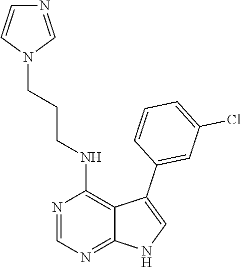 [SMILES: Clc1cccc(-c2c[nH]c3ncnc(NCCCn4ccnc4)c23)c1] |
| 57 | N1-(5-(3-chlorophenyl)-7H-pyrrolo[2,3- d]pyrimidin-4-yl)-N2,N2- dimethylethane-1,2-diamine | 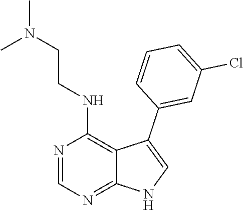 [SMILES: CN(C)CCNc1ncnc2[nH]cc(-c3cccc(Cl)c3)c12] |
| 58 | 5-(3-chlorophenyl)-N-(2-methoxyethyl)- 7H-pyrrolo[2,3-d]pyrimidin-4-amine | 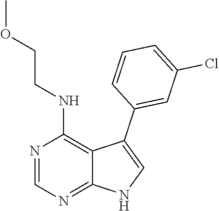 [SMILES: COCCNc1ncnc2[nH]cc(-c3cccc(Cl)c3)c12] |
| 59 | N-((1H-pyrazol-5-yl)methyl)-5-(3- chlorophenyl)-7H-pyrrolo[2,3- d]pyrimidin-4-amine | 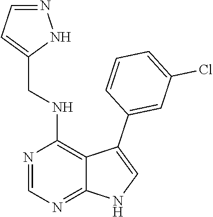 [SMILES: Clc1cccc(-c2c[nH]c3ncnc(NCc4ccn[nH]4)c23)c1] |
| 60 | 5-(3-chlorophenyl)-N- (cyclopentylmethyl)-7H-pyrrolo[2,3- d]pyrimidin-4-amine | 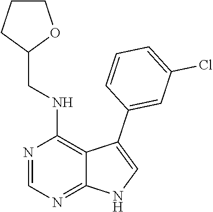 [SMILES: Clc1cccc(-c2c[nH]c3ncnc(NCC4CCCO4)c23)c1] |
| 61 | 5-(3-chlorophenyl)-N-((tetrahydro-2H- pyran-4-yl)methyl)-7H-pyrrolo[2,3- d]pyrimidin-4-amine | 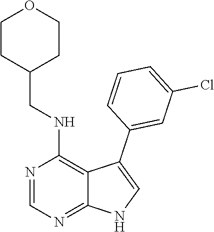 [SMILES: Clc1cccc(-c2c[nH]c3ncnc(NCC4CCOCC4)c23)c1] |
| 62 | 5-(3-chlorophenyl)-N-(furan-3- ylmethyl)-7H-pyrrolo[2,3-d]pyrimidin-4- amine | 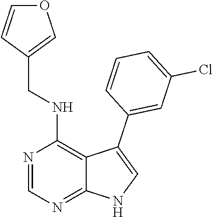 [SMILES: Clc1cccc(-c2c[nH]c3ncnc(NCc4ccoc4)c23)c1] |
| 63 | 5-(3-chlorophenyl)-N-(2-(pyridin-2- yl)ethyl)-7H-pyrrolo[2,3-d]pyrimidin-4- amine | 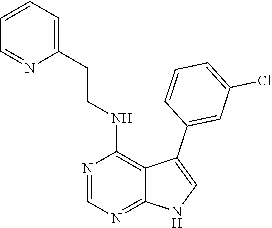 [SMILES: Clc1cccc(-c2c[nH]c3ncnc(NCCc4ccccn4)c23)c1] |
| 64 | 5-(3-chlorophenyl)-N-isopentyl-7H- pyrrolo[2,3-d]pyrimidin-4-amine | 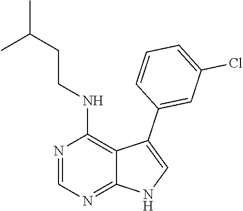 [SMILES: CC(C)CCNc1ncnc2[nH]cc(-c3cccc(Cl)c3)c12] |
| 65 | 5-(3-chlorophenyl)-N-(1- methylpiperidin-4-yl)-7H-pyrrolo[2,3- d]pyrimidin-4-amine | 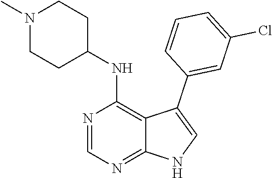 [SMILES: CN1CCC(Nc2ncnc3[nH]cc(-c4cccc(Cl)c4)c23)CC1] |
| 66 | 5-(3-chlorophenyl)-N-(2- morpholinoethyl)-7H-pyrrolo[2,3- d]pyrimidin-4-amine | 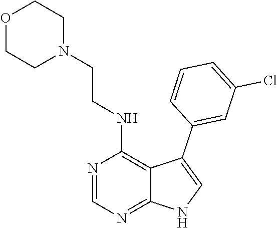 [SMILES: Clc1cccc(-c2c[nH]c3ncnc(NCCN4CCOCC4)c23)c1] |
| 67 | 5-(3-chlorophenyl)-N-(cyclopent-1-en-1- ylmethyl)-7H-pyrrolo[2,3-d]pyrimidin-4- amine | 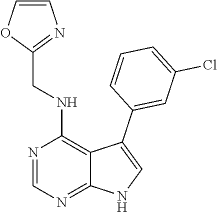 [SMILES: Clc1cccc(-c2c[nH]c3ncnc(NCc4ncco4)c23)c1] |
| 68 | 5-(3-chlorophenyl)-N-(2- methoxybenzyl)-7H-pyrrolo[2,3- d]pyrimidin-4-amine | 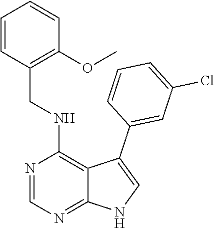 [SMILES: COc1ccccc1CNc1ncnc2[nH]cc(-c3cccc(Cl)c3)c12] |
| 69 | 5-(3-chlorophenyl)-N-(2-methylbenzyl)- 7H-pyrrolo[2,3-d]pyrimidin-4-amine | 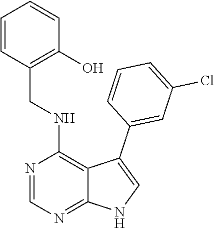 [SMILES: Oc1ccccc1CNc1ncnc2[nH]cc(-c3cccc(Cl)c3)c12] |
| 70 | 5-(3-chlorophenyl)-N-(cyclopent-1-en-1- ylmethyl)-7H-pyrrolo[2,3-d]pyrimidin-4- amine | 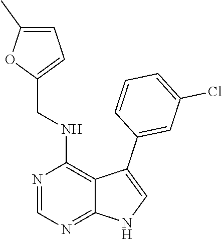 [SMILES: Cc1ccc(CNc2ncnc3[nH]cc(-c4cccc(Cl)c4)c23)o1] |
| 71 | 5-(3-chlorophenyl)-N-(pyridin-4- ylmethyl)-7H-pyrrolo[2,3-d]pyrimidin-4- amine | 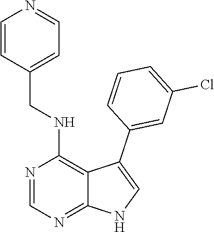 [SMILES: Clc1cccc(-c2c[nH]c3ncnc(NCc4ccncc4)c23)c1] |
| 72 | 5-(3-chlorophenyl)-N-(3,4- dimethoxyphenethyl)-7H-pyrrolo[2,3- d]pyrimidin-4-amine | 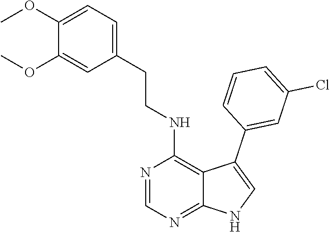 [SMILES: COc1ccc(CCNc2ncnc3[nH]cc(-c4cccc(Cl)c4)c23)cc1OC] |
| 73 | 5-(3-chlorophenyl)-N-(2,4- dimethoxybenzyl)-7H-pyrrolo[2,3- d]pyrimidin-4-amine | 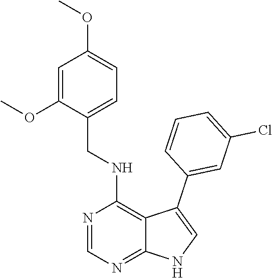 [SMILES: COc1ccc(CNc2ncnc3[nH]cc(-c4cccc(Cl)c4)c23)c(OC)c1] |
| 74 | 5-(3-chlorophenyl)-N-(3- methoxybenzyl)-7H-pyrrolo[2,3- d]pyrimidin-4-amine | 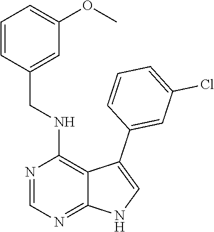 [SMILES: COc1cccc(CNc2ncnc3[nH]cc(-c4cccc(Cl)c4)c23)c1] |
| 75 | 5-(3-chlorophenyl)-N-(oxazol-5- ylmethyl)-7H-pyrrolo[2,3-d]pyrimidin-4- amine | 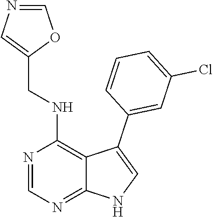 [SMILES: Clc1cccc(-c2c[nH]c3ncnc(NCc4cnco4)c23)c1] |
| 76 | N-((1H-imidazol-5-yl)methyl)-5-(3- chlorophenyl)-7H-pyrrolo[2,3- d]pyrimidin-4-amine | 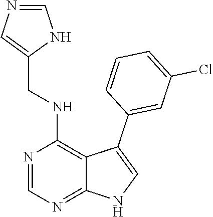 [SMILES: Clc1cccc(-c2c[nH]c3ncnc(NCc4cnc[nH]4)c23)c1] |
| 77 | 5-(3-chlorophenyl)-N-(2,4- dimethoxybenzyl)-7H-pyrrolo[2,3- d]pyrimidin-4-amine | 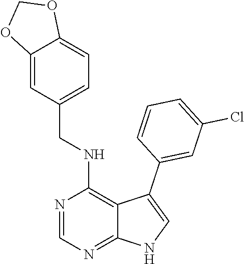 [SMILES: Clc1cccc(-c2c[nH]c3ncnc(NCc4ccc5c(c4)OCO5)c23)c1] |
| 78 | 4-(4-benzylcyclohexyl)-5-(3- chlorophenyl)-7H-pyrrolo[2,3- d]pyrimidine | 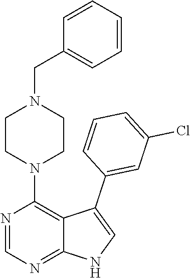 [SMILES: Clc1cccc(-c2c[nH]c3ncnc(N4CCN(Cc5ccccc5)CC4)c23)c1] |
| 79 | 5-(3-chlorophenyl)-N-(thiazol-2- ylmethyl)-7H-pyrrolo[2,3-d]pyrimidin-4- amine | 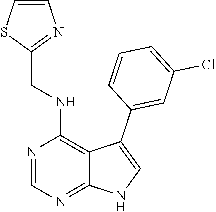 [SMILES: Clc1cccc(-c2c[nH]c3ncnc(NCc4nccs4)c23)c1] |
| 80 | 5-(3-chlorophenyl)-N-((1- ethylpyrrolidin-2-yl)methyl)-7H- pyrrolo[2,3-d]pyrimidin-4-amine | 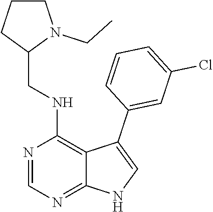 [SMILES: CCN1CCCC1CNc1ncnc2[nH]cc(-c3cccc(Cl)c3)c12] |
| 81 | 4-([1,4′-bipiperidin]-1′-yl)-5-(3- chlorophenyl)-7H-pyrrolo[2,3- d]pyrimidine | 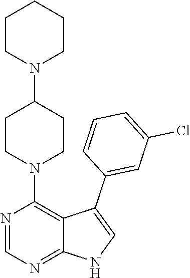 [SMILES: Clc1cccc(-c2c[nH]c3ncnc(N4CCC(N5CCCCC5)CC4)c23)c1] |
| 82 | N1-(5-(3-chlorophenyl)-7H-pyrrolo[2,3- d]pyrimidin-4-yl)-N2,N2-diethylethane- 1,2-diamine | 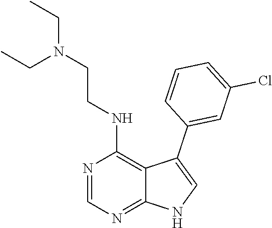 [SMILES: CCN(CC)CCNc1ncnc2[nH]cc(-c3cccc(Cl)c3)c12] |
| 83 | 5-(3-chlorophenyl)-N-(2-(pyrrolidin-1- yl)ethyl)-7H-pyrrolo[2,3-d]pyrimidin-4- amine | 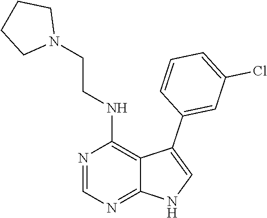 [SMILES: Clc1cccc(-c2c[nH]c3ncnc(NCCN4CCCC4)c23)c1] |
| 84 | 5-(3-chlorophenyl)-N- (cycloheptylmethyl)-7H-pyrrolo[2,3- d]pyrimidin-4-amine | 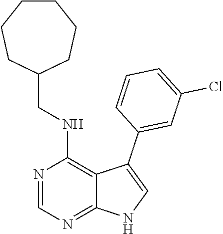 [SMILES: Clc1cccc(-c2c[nH]c3ncnc(NCC4CCCCCC4)c23)c1] |
| 85 | 5-(3-chlorophenyl)-N-(3,4- dimethylbenzyl)-7H-pyrrolo[2,3- d]pyrimidin-4-amine | 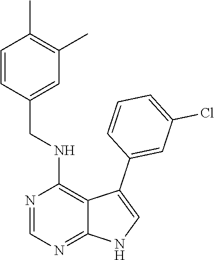 [SMILES: Cc1ccc(CNc2ncnc3[nH]cc(-c4cccc(Cl)c4)c23)cc1C] |
| 86 | 5-(3-chlorophenyl)-N-((6-chloropyridin- 3-yl)methyl)-7H-pyrrolo[2,3- d]pyrimidin-4-amine | 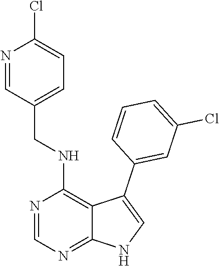 [SMILES: Clc1cccc(-c2c[nH]c3ncnc(NCc4ccc(Cl)nc4)c23)c1] |
| 87 | 5-(3-chlorophenyl)-4-(4-phenylpiperidin- 1-yl)-7H-pyrrolo[2,3-d]pyrimidine | 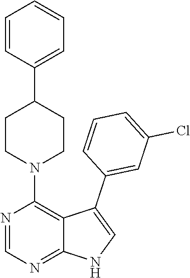 [SMILES: Clc1cccc(-c2c[nH]c3ncnc(N4CCC(c5ccccc5)CC4)c23)c1] |
| 88 | 5-(3-chlorophenyl)-N-(3,3- diphenylpropyl)-7H-pyrrolo[2,3- d]pyrimidin-4-amine | 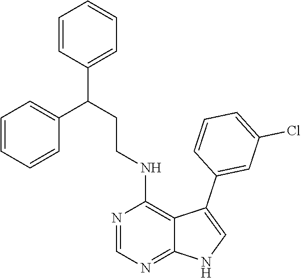 [SMILES: Clc1cccc(-c2c[nH]c3ncnc(NCCC(c4ccccc4)c4ccccc4)c23)c1] |
| 89 | 5-(3-chlorophenyl)-4-(4-phenylpiperazin- 1-yl)-7H-pyrrolo[2,3-d]pyrimidine | 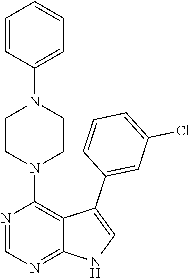 [SMILES: Clc1cccc(-c2c[nH]c3ncnc(N4CCN(c5ccccc5)CC4)c23)c1] |
| 90 | 4-(4-benzylpiperidin-1-yl)-5-(3- chlorophenyl)-7H-pyrrolo[2,3- d]pyrimidine | 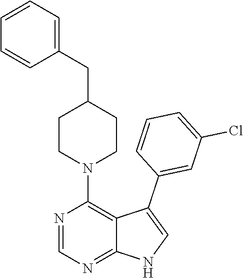 [SMILES: Clc1cccc(-c2c[nH]c3ncnc(N4CCC(Cc5ccccc5)CC4)c23)c1] |
| 91 | 5-(3-chlorophenyl)-N-(3-methylbenzyl)- 7H-pyrrolo[2,3-d]pyrimidin-4-amine | 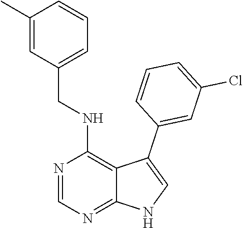 [SMILES: Cc1cccc(CNc2ncnc3[nH]cc(-c4cccc(Cl)c4)c23)c1] |
| 92 | 5-(3-chlorophenyl)-N-propyl-7H- pyrrolo[2,3-d]pyrimidin-4-amine | 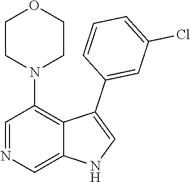 [SMILES: Clc1cccc(-c2c[nH]c3cncc(N4CCOCC4)c23)c1] |
| 93 | 4-(3-phenyl-1H-indol-4-yl)morpholine | 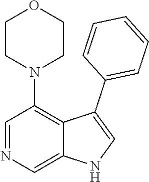 [SMILES: c1ccc(-c2c[nH]c3cncc(N4CCOCC4)c23)cc1] |
| 94 | 3-(3-chlorophenyl)-4- morpholinofuro[2,3-b]pyridine | 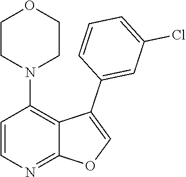 [SMILES: Clc1cccc(-c2coc3nccc(N4CCOCC4)c23)c1] |
| 95 | 4-(5-(3-chlorophenyl)-7-methyl-7H- pyrrolo[2,3-d]pyrimidin-4-yl)morpholine | 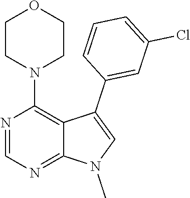 [SMILES: Cn1cc(-c2cccc(Cl)c2)c2c(N3CCOCC3)ncnc21] |
| 96 | 4-((5-(3-chlorophenyl)-4-morpholino- 7H-pyrrolo[2,3-d]pyrimidin-2- yl)amino)benzenesulfonamide | 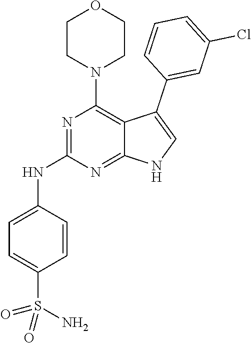 [SMILES: NS(=O)(=O)c1ccc(Nc2nc(N3CCOCC3)c3c(-c4cccc(Cl)c4)c[nH]c3n2)cc1] |
| 97 | 1-(5-(3-chlorophenyl)-4-morpholino-7H- pyrrolo[2,3-d]pyrimidin-2-yl)but-3-en-2- one | 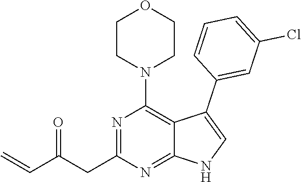 [SMILES: C=CC(=O)Cc1nc(N2CCOCC2)c2c(-c3cccc(Cl)c3)c[nH]c2n1] |
| 98 | 4-((5-(3-chlorophenyl)-4-morpholino- 7H-pyrrolo[2,3-d]pyrimidin-2-yl)amino)- N-methylbenzenesulfonamide | 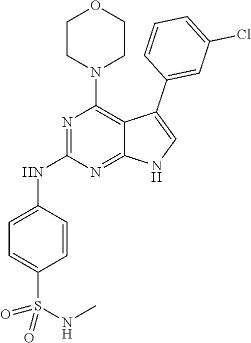 [SMILES: CNS(=O)(=O)c1ccc(Nc2nc(N3CCOCC3)c3c(-c4cccc(Cl)c4)c[nH]c3n2)cc1] |
| 99 | methyl 4-((5-(3-chlorophenyl)-4- morpholino-7H-pyrrolo[2,3-d]pyrimidin- 2-yl)amino)benzoate | 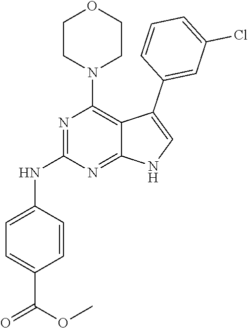 [SMILES: COC(=O)c1ccc(Nc2nc(N3CCOCC3)c3c(-c4cccc(Cl)c4)c[nH]c3n2)cc1] |
| 100 | 4-((5-(3-chlorophenyl)-4-morpholino- 7H-pyrrolo[2,3-d]pyrimidin-2- yl)amino)benzoic acid | 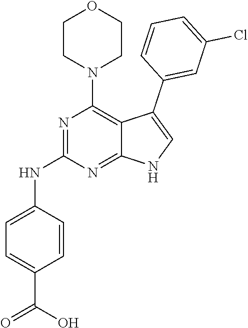 [SMILES: O=C(O)c1ccc(Nc2nc(N3CCOCC3)c3c(-c4cccc(Cl)c4)c[nH]c3n2)cc1] |
| 101 | 5-(3-chlorophenyl)-4-morpholino-N-(4- nitrophenyl)-7H-pyrrolo[2,3- d]pyrimidin-2-amine | 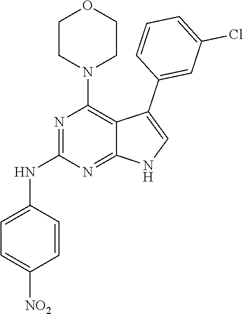 [SMILES: O=[N+]([O-])c1ccc(Nc2nc(N3CCOCC3)c3c(-c4cccc(Cl)c4)c[nH]c3n2)cc1] |
| 102 | 1-(5-(3-chlorophenyl)-7H-pyrrolo[2,3- d]pyrimidin-4-yl)-4-methylpiperidin-4- amine | 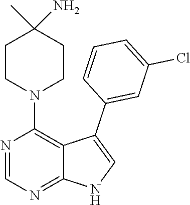 [SMILES: CC1(N)CCN(c2ncnc3[nH]cc(-c4cccc(Cl)c4)c23)CC1] |
| 103 | 1-(5-(3-chlorophenyl)-7H-pyrrolo[2,3- d]pyrimidin-4-yl)-4-methylpiperidin-4-ol | 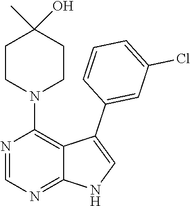 [SMILES: CC1(O)CCN(c2ncnc3[nH]cc(-c4cccc(Cl)c4)c23)CC1] |
| 104 | 1-((5-(3-chlorophenyl)-7H-pyrrolo[2,3- d]pyrimidin-4-yl)amino)propan-2-ol | 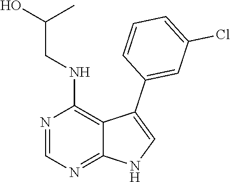 [SMILES: CC(O)CNc1ncnc2[nH]cc(-c3cccc(Cl)c3)c12] |
| 105 | N-(1-(5-(3-chlorophenyl)-7H- pyrrolo[2,3-d]pyrimidin-4-yl)piperidin-4- yl)acetamide | 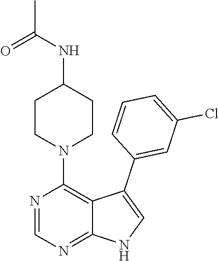 [SMILES: CC(=O)NC1CCN(c2ncnc3[nH]cc(-c4cccc(Cl)c4)c23)CC1] |
| 106 | 1-(5-(3-chlorophenyl)-7H-pyrrolo[2,3- d]pyrimidin-4-yl)-N-methylpiperidin-4- amine | 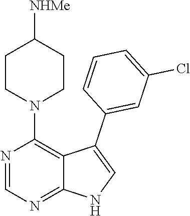 [SMILES: CNC1CCN(c2ncnc3[nH]cc(-c4cccc(Cl)c4)c23)CC1] |
| 107 | 1-(5-(3-chlorophenyl)-7H-pyrrolo[2,3- d]pyrimidin-4-yl)-N,N- dimethylpiperidin-4-amine | 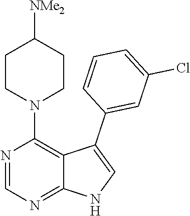 [SMILES: CN(C)C1CCN(c2ncnc3[nH]cc(-c4cccc(Cl)c4)c23)CC1] |
| 108 | 2-((5-(3-chlorophenyl)-7H-pyrrolo[2,3- d]pyrimidin-4-yl)(methyl)amino)ethan-1- ol | 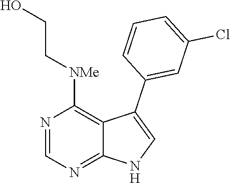 [SMILES: CN(CCO)c1ncnc2[nH]cc(-c3cccc(Cl)c3)c12] |
| 109 | 5-(3-chlorophenyl)-4-(4-methylpiperidin- 1-yl)-7H-pyrrolo[2,3-d]pyrimidine | 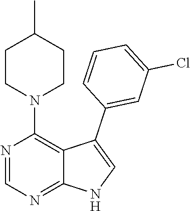 [SMILES: CC1CCN(c2ncnc3[nH]cc(-c4cccc(Cl)c4)c23)CC1] |
| 110 | 5-(3-chlorophenyl)-4-(4- methoxypiperidin-1-yl)-7H-pyrrolo[2,3- d]pyrimidine | 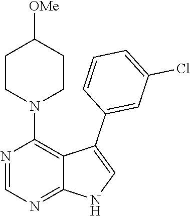 [SMILES: COC1CCN(c2ncnc3[nH]cc(-c4cccc(Cl)c4)c23)CC1] |
| 111 | 4-((5-(3-chlorophenyl)-4-(4- hydroxypiperidin-1-yl)-7H-pyrrolo[2,3- d]pyrimidin-2- yl)amino)benzenesulfonamide | 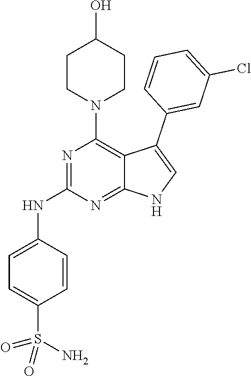 [SMILES: NS(=O)(=O)c1ccc(Nc2nc(N3CCC(O)CC3)c3c(-c4cccc(Cl)c4)c[nH]c3n2)cc1] |
| 112 | 4-((5-(3-chlorophenyl)-4-(4- hydroxypiperidin-1-yl)-7H-pyrrolo[2,3- d]pyrimidin-2-yl)amino)-N- methylbenzenesulfonamide | 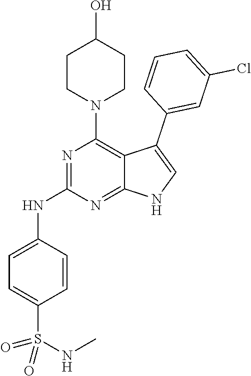 [SMILES: CNS(=O)(=O)c1ccc(Nc2nc(N3CCC(O)CC3)c3c(-c4cccc(Cl)c4)c[nH]c3n2)cc1] |
| 113 | 4-((5-(3-chlorophenyl)-4-(4- hydroxypiperidin-1-yl)-7H-pyrrolo[2,3- d]pyrimidin-2-yl)amino)benzoic acid | 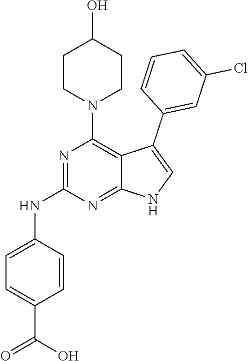 [SMILES: O=C(O)c1ccc(Nc2nc(N3CCC(O)CC3)c3c(-c4cccc(Cl)c4)c[nH]c3n2)cc1] |
| 114 | 2-((5-(3-chlorophenyl)-7H-pyrrolo[2,3- d]pyrimidin-4-yl)(ethyl)amino)ethan-1-ol | 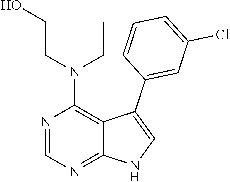 [SMILES: CCN(CCO)c1ncnc2[nH]cc(-c3cccc(Cl)c3)c12] |
| 115 | 5-(3-chlorophenyl)-N-methyl-N-propyl- 7H-pyrrolo[2,3-d]pyrimidin-4-amine | 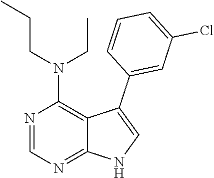 [SMILES: CCCN(CC)c1ncnc2[nH]cc(-c3cccc(Cl)c3)c12] |
| 116 | N-butyl-5-(3-chlorophenyl)-7H- pyrrolo[2,3-d]pyrimidin-4-amine | 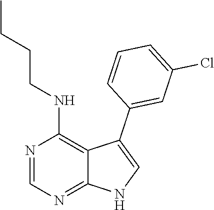 [SMILES: CCCCNc1ncnc2[nH]cc(-c3cccc(Cl)c3)c12] |
| 117 | 3-((5-(3-chlorophenyl)-7H-pyrrolo[2,3- d]pyrimidin-4-yl)amino)-2- methylpropan-1-ol | 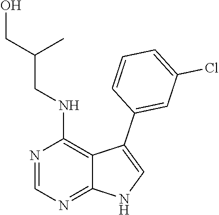 [SMILES: CC(CO)CNc1ncnc2[nH]cc(-c3cccc(Cl)c3)c12] |
| 118 | 1-(5-(3-chlorophenyl)-7H-pyrrolo[2,3- d]pyrimidin-4-yl)piperidin-4-one | 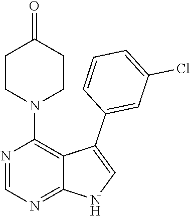 [SMILES: O=C1CCN(c2ncnc3[nH]cc(-c4cccc(Cl)c4)c23)CC1] |
| 119 | 3-((5-(3-chlorophenyl)-7H-pyrrolo[2,3- d]pyrimidin-4-yl)amino)propan-1-ol | 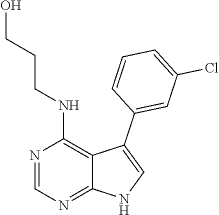 [SMILES: OCCCNc1ncnc2[nH]cc(-c3cccc(Cl)c3)c12] |
| 120 | 4-((5-(3-chlorophenyl)-4-morpholino- 7H-pyrrolo[2,3-d]pyrimidin-2- yl)amino)benzonitrile | 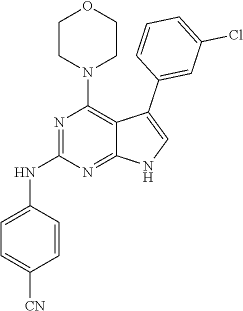 [SMILES: N#Cc1ccc(Nc2nc(N3CCOCC3)c3c(-c4cccc(Cl)c4)c[nH]c3n2)cc1] |
| 121 | 3-((5-(3-chlorophenyl)-7H-pyrrolo[2,3- d]pyrimidin-4-yl)(methyl)amino)-2- methylpropan-1-ol | 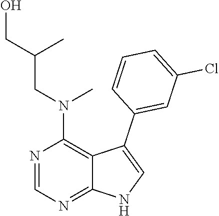 [SMILES: CC(CO)CN(C)c1ncnc2[nH]cc(-c3cccc(Cl)c3)c12] |
| 122 | 5-(3-chlorophenyl)-N-(3-methoxy-2- methylpropyl)-N-methyl-7H-pyrrolo[2,3- d]pyrimidin-4-amine | 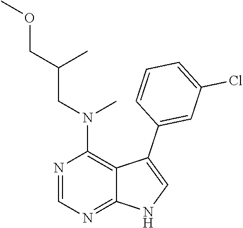 [SMILES: COCC(C)CN(C)c1ncnc2[nH]cc(-c3cccc(Cl)c3)c12] |
| 123 | 5-(3-chlorophenyl)-N-(3-methoxy-2- methylpropyl)-7H-pyrrolo[2,3- d]pyrimidin-4-amine | 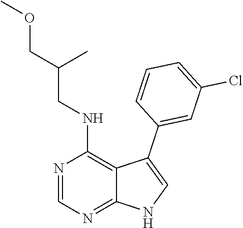 [SMILES: COCC(C)CNc1ncnc2[nH]cc(-c3cccc(Cl)c3)c12] |
| 124 | N-(1-(5-(3-chlorophenyl)-7H- pyrrolo[2,3-d]pyrimidin-4-yl)-4- methylpiperidin-4-yl)acetamide | 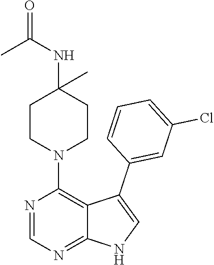 [SMILES: CC(=O)NC1(C)CCN(c2ncnc3[nH]cc(-c4cccc(Cl)c4)c23)CC1] |
| 125 | 3-((5-(3-chlorophenyl)-7H-pyrrolo[2,3- d]pyrimidin-4-yl)amino)-2- methylpropanamide | 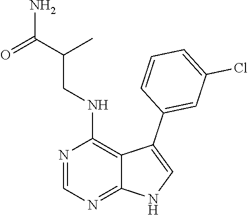 [SMILES: CC(CNc1ncnc2[nH]cc(-c3cccc(Cl)c3)c12)C(N)=O] |
| 126 | 3-((5-(3-chlorophenyl)-7H-pyrrolo[2,3- d]pyrimidin-4-yl)amino)-2- methylpropanoic acid | 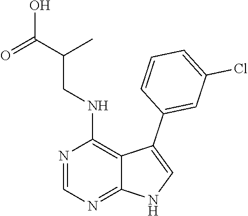 [SMILES: CC(CNc1ncnc2[nH]cc(-c3cccc(Cl)c3)c12)C(=O)O] |
| 127 | N¹-(5-(3-chlorophenyl)-7H-pyrrolo[2,3- d]pyrimidin-4-yl)-2-methylpropane-1,3- diamine | 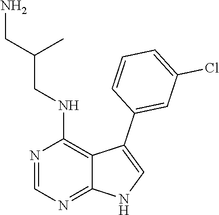 [SMILES: CC(CN)CNc1ncnc2[nH]cc(-c3cccc(Cl)c3)c12] |
| 128 | N-(3-((5-(3-chlorophenyl)-7-((2- (trimethylsilyl)ethoxy)methyl)-7H- pyrrolo[2,3-d]pyrimidin-4-yl)amino)-2- methylpropyl)acetamide | 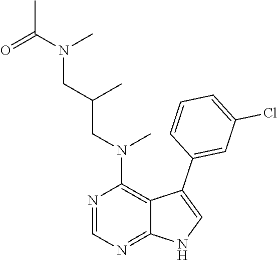 [SMILES: CC(=O)N(C)CC(C)CN(C)c1ncnc2[nH]cc(-c3cccc(Cl)c3)c12] |
| 129 | N-(3-((5-(3-chlorophenyl)-7H- pyrrolo[2,3-d]pyrimidin-4-yl)amino)-2- methylpropyl)acetamide | 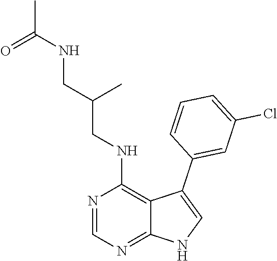 [SMILES: CC(=O)NCC(C)CNc1ncnc2[nH]cc(-c3cccc(Cl)c3)c12] |
| 130 | N-(2-methyl-3-((5-phenyl-7H- pyrrolo[2,3-d]pyrimidin-4- yl)amino)propyl)acetamide | 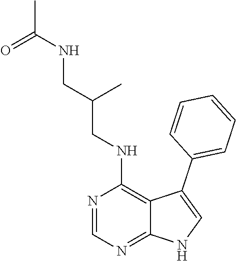 [SMILES: CC(=O)NCC(C)CNc1ncnc2[nH]cc(-c3ccccc3)c12] |
| 131 | N¹-(5-(3-chlorophenyl)-7H-pyrrolo[2,3- d]pyrimidin-4-yl)-N³,2-dimethylpropane- 1,3-diamine | 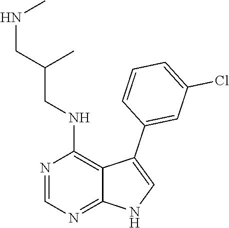 [SMILES: CNCC(C)CNc1ncnc2[nH]cc(-c3cccc(Cl)c3)c12] |
| 132 | N¹,N³-bis(5-(3-chlorophenyl)-7H- pyrrolo[2,3-d]pyrimidin-4-yl)-2- methylpropane-1,3-diamine | 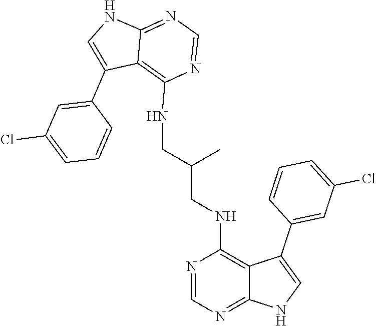 [SMILES: CC(CNc1ncnc2[nH]cc(-c3cccc(Cl)c3)c12)CNc1ncnc2[nH]cc(-c3cccc(Cl)c3)c12] |

Any combination of the groups described above for the various variables is contemplated herein. Throughout the specification, groups and substituents thereof are chosen by one skilled in the field to provide stable moieties and compounds.

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively, Compounds described herein, and the pharmaceutically acceptable salts, solvates, or stereoisomers thereof which contain the aforementioned isotopes and/or other isotopes of other atoms are within the scope of this invention, Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxy benzoate, γ-hydroxy butyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl) benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid. 2-naphthalenesulfonic acid, 4-methylbicyclo-|2.2.2|oct-2-ene-1-carboxylic acid, glucoheptonic acid. 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate. N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

It is understood that other analogous procedures and reagents could be used, and that these Schemes are only meant as non-limiting examples.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow; the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below; as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —OR^{a} where R^{a} is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe. —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl), Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₈ cycloalkyl or C₃-C₈ cycloalkenyl), or three to four carbon atoms (C₃-C₄ cycloalkyl or C₃-C₄ cycloalkenyl), In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl. 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Cyanoalkyl” refers to an alkyl radical, as defined above, that is substituted by one or more cyano group. In some embodiments, the alkyl is substituted with one cyano. In some embodiments, the alkyl is substituted with one or two cyanos. Cyanoalkyls include, for example, cyanomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen (e.g., NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH (CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized: the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₈ heterocycloalkenyl), or two to four carbon atoms (C₂-C₄ heterocycloalkyl or C₂-C₄ heterocycloalkenyl), Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized: the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1.4]dioxepinyl, 1,4-benzodioxanyl, benzonaphthofuranyl, benzoxazolyl, benzodioxolyl, benzodioxinyl, benzopyranyl, benzopyranonyl.

benzofuranyl, benzofuranonyl, benzothienyl (benzothiophenyl), benzotriazolyl, benzo[4.6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl), Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example. “optionally substituted alkyl” means either “alkyl” or “substituted alkyl” as defined above. Further, an optionally substituted group may be un-substituted (e.g., CH₂CH₃), fully substituted (e.g., CF₂CF₃), mono-substituted (e.g., CH₂CH₂F) or substituted at a level anywhere in-between fully substituted and mono-substituted (e.g., CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1.000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

### Methods of Use

Disclosed herein is a method of modulating the activity serine/threonine protein kinase 3 (STK3) or serine/threonine protein kinase 4 (STK4) or both in a mammal, comprising administering to the mammal a compound disclosed herein, or a pharmaceutically acceptable salt thereof.

In some embodiments, modulating comprises inhibiting.

In some embodiments, the mammal has a disease or condition that would benefit from inhibition of STK3 or STK4 or both. In some embodiments, the mammal is a human.

In some embodiments, the disease or condition is cancer.

In some embodiments, the cancer is a blood cancer. In some embodiments, is leukemia, lymphoma, myeloma, myeloproliferative neoplasms (MPNs), or myelodysplastic syndromes (MDS), In some embodiments, the leukemia is acute lymphoblastic leukemia (ALL), acute myeloid leukemia (AML), chronic lymphocytic leukemia (CLL), chronic myeloid leukemia (CML), hairy cell leukemia (HCL), chronic myelomonocytic leukemia (CMML), large granular lymphocytic (LGL), blastic plasmacytoid dendritic cell neoplasm (BPDCN), B-cell prolymphocytic leukemia (B-PLL), or T-cell prolymphocytic leukemia (T-PLL).

In some embodiments, the lymphoma is Hodgkin Lymphoma (HL) or Non-Hodgkin Lymphoma (NHL).

In some embodiments, the myeloma is multiple myeloma, or plasmacytoma.

In some embodiments, the myeloproliferative neoplasms is myelofibrosis, polycythemia vera or essential thrombocythemia.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A, and Lachman, L., Eds., Pharmaceutical Dosage Forms. Marcel Decker, New York. N.Y., 1980; and Pharmaceutical Dosage Forms and Drug Delivery Systems. Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin.

In another aspect, the compounds disclosed herein are administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned aspects are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

### EXAMPLES

### Preparation of Compounds

All reagents were purchased from commercial sources and used without further purification unless otherwise stated. Chromatographic purification was carried out using WelFlash SiO₂-II or irregular silica gel (40-63 μm) column cartridges and eluted using a Biotage Sweden AB box B. Compound purification by preparative HPLC was performed on a Waters auto purification system with an ESI mass spectrometer (Waters 2555+2767+2489+SQD) using the following conditions; Welch Utimate XB—C₁₈ column (reverse phase, 50_150 mm, 5 μm); Mobile phases: water doped with 0.1% formic acid and acetonitrile.

### Example 1a: Synthesis of 4-(5-Iodo-7-((2-(trimethylsilyl)ethoxy)methyl)-7H-pyrrolo[2,3-d]pyrimidin-4-yl)morpholine (Common Intermediate 1)

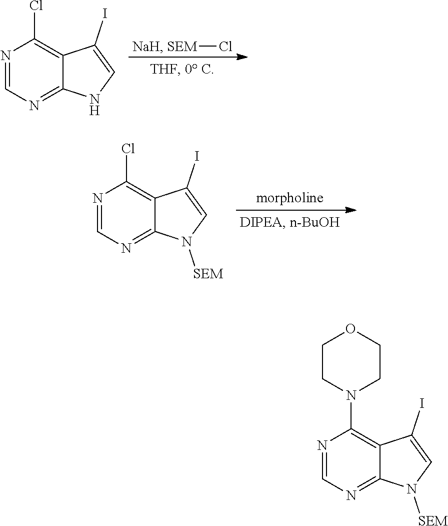

### Step 1

To a solution of 4-chloro-5-iodo-7H-pyrrolo[2,3-d]pyrimidine (25 g, 89.6 mmol) in THF (300 mL) was added NaH (4.3 g, 107.5 mmol, 60% in oil) portion wise at 0° C. under N₂. The mixture was stirred at 0° C. for 30 min and 2-(trimethylsilyl)ethoxymethyl chloride (17.8 g, 107.5 mmol) was added dropwise at 0° C. The resulting mixture was stirred at room temperature overnight. The reaction mixture was then quenched with ice water (100 mL) and extracted with ethyl acetate (60 mL×3). The organic layer was washed with water (50 mL×3) and brine (50 mL), dried over Na₂SO₄, and concentrated. The crude product was purified by flash column chromatography to afford 4-chloro-5-iodo-7-((2-(trimethylsilyl)ethoxy)methyl)-7H-pyrrolo[2,3-d]pyrimidine (19 g, 52%) as an oil. HPLC/UV purity: 90%; LC-MS (ESI): 410.1 [M+H]⁺.

### Step 2

The mixture of 4-chloro-5-iodo-7-((2-(trimethylsilyl)ethoxy)methyl)-7H-pyrrolo[2,3-d]pyrimidine (4.09 g, 10 mmol), morpholine (1.044 g, 12 mmol) and DIPEA (2.58 mg, 20 mmol) in n-BuOH (100 mL) was heated at 80° C. for 3 h. The reaction mixture was cooled to room temperature and the solvent was removed in vacuo. The residue was diluted with EtOAc (40 mL), washed with water (20 mL×3) and brine (10 mL), dried over Na₂SO₄, and concentrated. The crude product was purified by flash column chromatography to afford 4-(5-iodo-7-((2-(trimethylsilyl)ethoxy) methyl)-7H-pyrrolo[2,3-d]pyrimidin-4-yl)morpholine (4.03 g, 88%) as a white solid. HPLC/UV purity: 90%; LC-MS (ESI): 461.1 [M+H]⁺.

### Example 1b: Synthesis of 4-(5-(p-tolyl)-7H-pyrrolo[2,3-d]pyrimidin-4-yl)morpholine (Compound 9) Starting from Common Intermediate 1

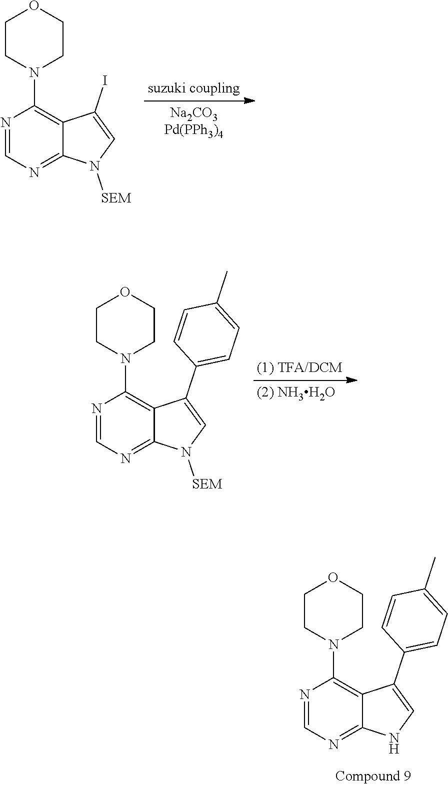 [SMILES: C[Si](C)(C)CCOCn1cc(I)c2c(N3CCOCC3)ncnc21 ; Cc1ccc(-c2cn(COCC[Si](C)(C)C)c3ncnc(N4CCOCC4)c23)cc1 ; Cc1ccc(-c2c[nH]c3ncnc(N4CCOCC4)c23)cc1]

### Step 1

The mixture of 4-(5-iodo-7-((2-(trimethylsilyl)ethoxy)methyl)-7H-pyrrolo[2,3-d]pyrimidin-4-yl)morpholine (230 mg, 0.5 mmol), p-tolylboronic acid (136 mg, 1 mmol), Pd(PPh₃)₄ (58 mg, 0.1 mmol) and Na₂CO₃ (212 mg, 2 mmol) in 1,4-dioxane (10 mL) and H₂O (1 mL) was heated at 85° C. for 18 h under N₂ atmosphere. The reaction mixture was cooled to room temperature and filtered through a pad of celite and concentrated in vacuo. The crude residue was then diluted with EtOAc (30 mL), washed by water and brine, dried over Na₂SO₄, and concentrated. The crude product was purified by Prep-TLC to afford 4-(5-(p-tolyl)-7-((2-(trimethylsilyl)ethoxy)methyl)-7H-pyrrolo[2,3-d]pyrimidin-4-yl)morpholine (140 mg, 66%) as a white solid. HPLC/UV purity: 95%; LC-MS (ESI): 425.2 [M+H]⁺.

### Step 2

The mixture of 4-(5-(p-tolyl)-7-((2-(trimethylsilyl)ethoxy)methyl)-7H-pyrrolo[2,3-d]pyrimidin-4-yl)morpholine (127 mg, 0.3 mmol) and TFA (2.5 mL) in DCM (5 mL) was stirred at room temperature for 2 h. The reaction mixture was then concentrated in vacuo to give an intermediate, which was diluted with a mixture of aqueous ammonia (2 mL), DCM (5 mL) and methanol (5 mL). The resulting mixture was stirred at room temperature for 18 h and concentrated in vacuo to give a crude product which washed by methanol (5 mL) and ether (10 mL) to afford 4-(5-(p-tolyl)-7H-pyrrolo[2,3-d]pyrimidin-4-yl)morpholine (70 mg, 80%) as a white solid. HPLC/UV purity: 96%; LC-MS (ESI): 295.1 [M+H]⁺. NMR (400 MHZ, DMSO-d₆): δ 12.08 (s, 1H), 8.34 (s, 1H), 7.41 (d, J=8.1 Hz, 2H), 7.25 (d, J=7.7 Hz, 2H), 3.43 (t, J=4.6 Hz, 4H), 3.16 (t, J=4.7 Hz, 4H), 2.34 (s, 3H).

The following compounds were prepared using analogous procedures to Example 1a and b.

| Name | Structure | Characterization |
|---|---|---|
| 4-(5-Phenyl-7H- pyrrolo[2,3-d]pyrimidin-4- yl)morpholine (Compound 1) | 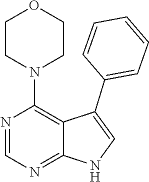 [SMILES: c1ccc(-c2c[nH]c3ncnc(N4CCOCC4)c23)cc1] | HPLC/UV purity: 100%; LC-MS (ESI): 281.1 [M + H] ⁺. ¹H NMR (400 MHz, DMSO-d₆): δ 12.52 (s, 1H), 8.44 (s, 1H), 7.55 (d, J = 2.1 Hz, 1H), 7.52-7.42 (m, 4H), 7.36-7.28 (m, 1H), 3.44 (t, J = 4.6 Hz, 4H), 3.24 (t, J = 4.6 Hz, 4H). |
| 4-(5-(o-Tolyl)-7H- pyrrolo[2,3-d]pyrimidin-4- yl)morpholine (Compound 2) | 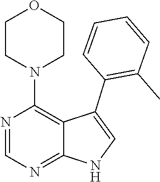 [SMILES: Cc1ccccc1-c1c[nH]c2ncnc(N3CCOCC3)c12] | HPLC/UV purity: 100%; LC-MS (ESI): 295.1 [M + H] ⁺. ¹H NMR (400 MHz, CDCl₃): δ 11.89 (s, 1H), 8.46 (s, 1H), 7.30-7.17 (m, 3H), 7.11 (s, 1H), 3.35 (t, J = 4.4 Hz, 4H), 3.25 (t, J = 4.4 Hz, 4H), 2.23 (s, 3H). |
| 4-(5-(2-Fluorophenyl)-7H- pyrrolo[2,3-d]pyrimidin-4- yl)morpholine (Compound 3) | 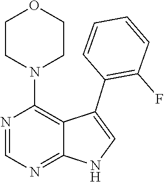 [SMILES: Fc1ccccc1-c1c[nH]c2ncnc(N3CCOCC3)c12] | HPLC/UV purity: 100%; LC-MS (ESI): 299.1 [M + H] ⁺. ¹H NMR (400 MHz, DMSO-d₆): δ 12.22 (s, 1H), 8.35 (s, 1H), 7.53-7.44 (m, 2H), 7.43-7.34 (m, 1H), 7.34-7.25 (m, 2H), 3.35 (t, J = 4.1 Hz, 4H), 3.14 (t, J = 4.5 Hz, 4H). |
| 4-(5-(2-Methoxyphenyl)- 7H-pyrrolo[2,3- d]pyrimidin-4- yl)morpholine (Compound 4) | 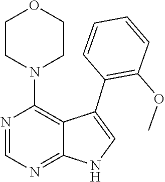 [SMILES: COc1ccccc1-c1c[nH]c2ncnc(N3CCOCC3)c12] | HPLC/UV purity: 100%; LC-MS (ESI): 311.2 [M + H] ⁺. ¹H NMR (400 MHz, DMSO-d₆): δ 12.48 (s, 1H), 8.39 (s, 1H), 7.39 (d, J = 2.2 Hz, 1H), 7.36 (td, J = 7.9, 1.8 Hz, 1H), 7.30 (dd, J = 7.6, 1.8 Hz, 1H), 7.10 (d, J = 8.2 Hz, 1H), 7.03 (t, J = 7.4 Hz, 1H), 3.74 (s, 3H), 3.35 (t, J = 4.5 Hz, 4H), 3.26 (t, J = 4.5 Hz, 4H). |
| 4-(5-(m-Tolyl)-7H- pyrrolo[2,3-d]pyrimidin-4- yl)morpholine (Compound 5) | 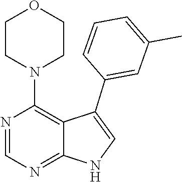 [SMILES: Cc1cccc(-c2c[nH]c3ncnc(N4CCOCC4)c23)c1] | HPLC/UV purity: 100%; LC-MS (ESI): 295.1 [M + H] ⁺. ¹H NMR (400 MHz, DMSO-d₆): δ 12.10 (s, 1H), 8.35 (s, 1H), 7.45 (d, J = 2.3 Hz, 1H), 7.35-7.30 (m, 3H), 7.15-7.06 (m, 1H), 3.43 (t, J = 4.7 Hz, 4H), 3.15 (t, J = 4.7 Hz, 4H), 2.38 (s, 3H). HRMS (ESI-TOF) calculated for C₁₇H₁₉N₄O [M + H]+: 295.1559; found 295.1548 |
| 4-(5-(3-Chlorophenyl)-7H- pyrrolo[2,3-d]pyrimidin-4- yl)morpholine (Compound 6) | 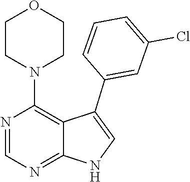 [SMILES: Clc1cccc(-c2c[nH]c3ncnc(N4CCOCC4)c23)c1] | HPLC/UV purity: 100%; LC-MS (ESI): 315.2 [M + H] ⁺. ¹H NMR (400 MHz, DMSO-d₆): δ 12.54 (s, 1H), 8.45 (s, 1H), 7.68 (d, J = 2.2 Hz, 1H), 7.59 (t, J = 1.8 Hz, 1H), 7.53-7.45 (m, 2H), 7.37 (dt, J = 6.9, 1.9 Hz, 1H), 3.49 (t, J = 4.6 Hz, 4H), 3.22 (t, J = 4.7 Hz, 4H). HRMS (ESI-TOF) calculated for C₁₆H₁₆ClN₄O [M + H]+: 315.1013; found 315.1013 |
| 4-(5-(3-Fluorophenyl)-7H- pyrrolo[2,3-d]pyrimidin-4- yl)morpholine (Compound 7) | 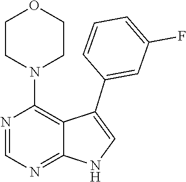 [SMILES: Fc1cccc(-c2c[nH]c3ncnc(N4CCOCC4)c23)c1] | HPLC/UV purity: 99%; LC-MS (ESI): 299.1 [M + H] ⁺. ¹H NMR (400 MHz, DMSO-d₆): δ 12.24 (s, 1H), 8.38 (s, 1H), 7.60 (s, 1H), 7.49 (q, J = 7.6 Hz, 1H), 7.41-7.32 (m, 2H), 7.12 (t, J = 7.3 Hz, 1H), 3.47 (t, J = 4.6 Hz, 4H), 3.16 (t, J = 4.6 Hz, 4H). |
| 4-(5-(3-Methoxyphenyl)- 7H-pyrrolo[2,3- d]pyrimidin-4- yl)morpholine (Compound 8) | 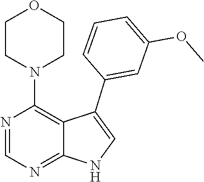 [SMILES: COc1cccc(-c2c[nH]c3ncnc(N4CCOCC4)c23)c1] | HPLC/UV purity: 100%; LC-MS (ESI): 311.1 [M + H] ⁺. ¹H NMR (400 MHz, DMSO-d₆): δ 12.50 (s, 1H), 8.43 (s, 1H), 7.57 (d, J = 2.2 Hz, 1H), 7.37 (t, J = 7.8 Hz, 1H), 7.11-7.03 (m, 2H), 6.90 (dd, J = 8.0, 2.4 Hz, 1H), 3.82 (s, 3H), 3.46 (t, J = 4.6 Hz, 4H), 3.25 (t, J = 4.8 Hz, 4H). |
| 4-(5-(4-Fluorophenyl)-7H- pyrrolo[2,3-d]pyrimidin-4- yl)morpholine (Compound 10) | 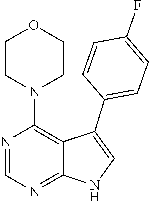 [SMILES: Fc1ccc(-c2c[nH]c3ncnc(N4CCOCC4)c23)cc1] | HPLC/UV purity: 98%; LC-MS (ESI): 299.1 [M + H] ⁺. ¹H NMR (400 MHz, DMSO-d₆): δ 12.15 (s, 1H), 8.36 (s, 1H), 7.54 (dd, J = 8.6, 5.7 Hz, 2H), 7.48 (d, J = 2.2 Hz, 1H), 7.28 (t, J = 8.8 Hz, 2H), 3.45 (t, J = 4.6 Hz, 4H), 3.15 (t, J = 4.5 Hz, 4H). |
| 3-(4-Morpholino-7H- pyrrolo[2,3-d]pyrimidin-5- yl)benzamide (Compound 11) | 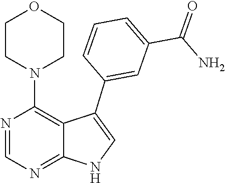 [SMILES: NC(=O)c1cccc(-c2c[nH]c3ncnc(N4CCOCC4)c23)c1] | HPLC/UV purity: 100%; LC-MS (ESI): 324.1 [M + H] ⁺. ¹H NMR (400 MHz, DMSO-d₆): δ 8.38 (s, 1H), 8.09 (s, 1H), 8.04 (s, 1H), 7.80 (d, J = 7.8 Hz, 1H), 7.69 (d, J = 7.8 Hz, 1H), 7.58 (s, 1H), 7.52 (t, J = 7.7 Hz, 1H), 7.40 (s, 1H), 3.42 (t, J = 4.5 Hz, 4H), 3.14 (t, J = 4.2 Hz, 4H). HRMS (ESI-TOF) calculated for C₁₇H₁₈N₅O₂ [M + H]+: 324.1460; found 324.1450. |
| 4-(5-(Thiophen-2-yl)-7H- pyrrolo[2,3-d]pyrimidin-4- yl)morpholine (Compound 12) | 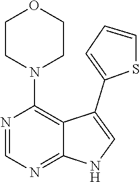 [SMILES: c1csc(-c2c[nH]c3ncnc(N4CCOCC4)c23)c1] | HPLC/UV purity: 100%; LC-MS (ESI): 287.1 [M + H] ⁺. ¹H NMR (400 MHz, DMSO-d₆): δ 12.20 (s, 1H), 8.35 (s, 1H), 7.49 (dd, J = 5.1, 1.3 Hz, 1H), 7.48 (s, 1H), 7.18-7.09 (m, 2H), 3.49 (t, J = 4.4 Hz, 4H), 3.21 (t, J = 4.4 Hz, 4H). |
| 4-(5-(Furan-2-yl)-7H- pyrrolo[2,3-d]pyrimidin-4- yl)morpholine (Compound 13) | 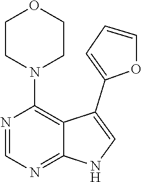 [SMILES: c1coc(-c2c[nH]c3ncnc(N4CCOCC4)c23)c1] | HPLC/UV purity: 100%; LC-MS (ESI) 271.1 [M + H] ⁺. ¹H NMR (400 MHz, DMSO-d₆): δ 12.21 (s, 1H), 8.33 (s, 1H), 7.74 (d, J = 2.9 Hz, 1H), 7.53 (d, J = 2.3 Hz, 1H), 6.57 (dd, J = 3.2, 1.9 Hz, 1H), 6.53 (d, J = 3.3 Hz, 1H), 3.57 (t, J = 4.8 Hz, 4H), 3.25 (t, J = 4.6 Hz, 4H). |
| 4-(5-(Furan-3-yl)-7H- pyrrolo[2,3-d]pyrimidin-4- yl)morpholine (Compound 14) | 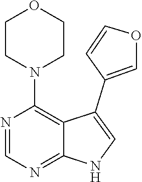 [SMILES: c1nc(N2CCOCC2)c2c(-c3ccoc3)c[nH]c2n1] | HPLC/UV purity: 100%; LC-MS (ESI): 271.1 [M + H] ⁺. ¹H NMR (400 MHz, DMSO-d₆): δ 12.21 (s, 1H), 8.36 (s, 1H), 7.85-7.83 (m, 1H), 7.75 (t, J = 1.9 Hz, 1H), 7.46 (d, J = 2.4 Hz, 1H), 6.72 (dd, J = 1.9, 0.9 Hz, 1H), 3.59 (t, J = 4.7 Hz, 4H), 3.29 (t, J = 4.7 Hz, 4H). |
| 4-(5-(2-Chlorophenyl)-7H- pyrrolo[2,3-d]pyrimidin-4- yl)morpholine (Compound 24) | 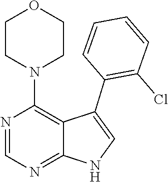 [SMILES: Clc1ccccc1-c1c[nH]c2ncnc(N3CCOCC3)c12] | HPLC/UV purity: 100%; LC-MS (ESI): 315.1 [M + H] ⁺. ¹H NMR (400 MHz, DMSO-d₆): δ 8.42 (s, 1H), 7.61-7.57 (m, 1H), 7.49 (d, J = 1.7 Hz, 1H), 7.47-7.37 (m, 4H), 3.31 (t, J = 4.6 Hz, 4H), 3.20 (t, J = 4.6 Hz, 4H). |
| 4-(5-(4-Chlorophenyl)-7H- pyrrolo[2,3-d]pyrimidin-4- yl)morpholine (Compound 25) | 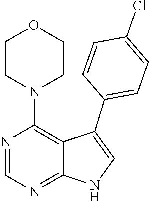 [SMILES: Clc1ccc(-c2c[nH]c3ncnc(N4CCOCC4)c23)cc1] | HPLC/UV purity: 100%; LC-MS (ESI): 315.1 [M + H] ⁺. ¹H NMR (400 MHz, DMSO-d₆): δ 12.21 (s, 1H), 8.37 (s, 1H), 7.57-7.48 (m, 5H), 3.47 (t, J = 4.7 Hz, 4H), 3.16 (t, J = 4.5 Hz, 4H). |

### Example 2a Synthesis of 4-chloro-5-(3-chlorophenyl)-7-((2-(trimethylsilyl)ethoxy) methyl)-7H-pyrrolo[2,3-d]pyrimidine (Common Intermediate 2)

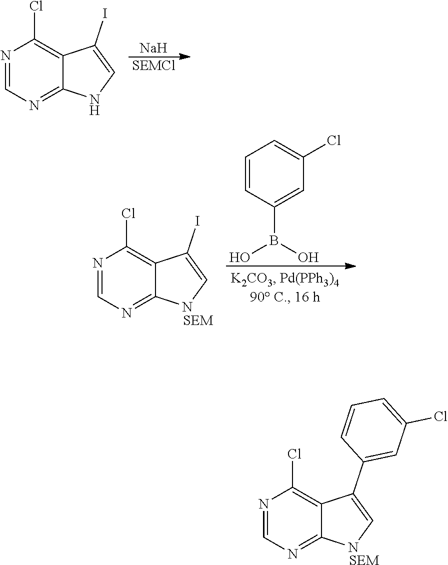

### 4-chloro-5-iodo-7-((2-(trimethylsilyl)ethoxy)methyl)-7H-pyrrolo[2,3-d]pyrimidine

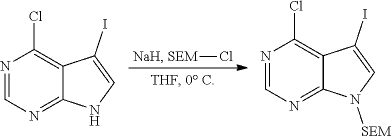

To a solution of 4-chloro-5-iodo-7H-pyrrolo[2,3-d]pyrimidine (10 g, 35.8 mmol) in THF (50 mL) was added NaH (1.7 g, 43.0 mmol, 60% in oil) portion wise at 0° C. under N₂. Then the mixture was stirred at 0° C. for 30 min, and 2-(Trimethylsilyl)ethoxymethyl chloride (7.2 g, 43.0 mmol) was added drop wise at 0° C. The mixture was stirred at room temperature overnight. The reaction mixture was quenched with ice water (50 mL), and then extracted with EA (30 mL×3). The organic layer was washed with water (30 mL×3) and brine (30 mL), dried over Na₂SO₄, concentrated and purified by flash column chromatography to afford 4-chloro-5-iodo-7-((2-(trimethylsilyl)ethoxy)methyl)-7H-pyrrolo[2,3-d]pyrimidine (6.5 g, 89%) as a yellow solid. HPLC/UV purity: 95%; LC-MS (ESI): 410.1 (M+1)+.

### 4-chloro-5-(3-chlorophenyl)-7-((2-(trimethylsilyl)ethoxy)methyl)-7H-pyrrolo[2,3-d]pyrimidine

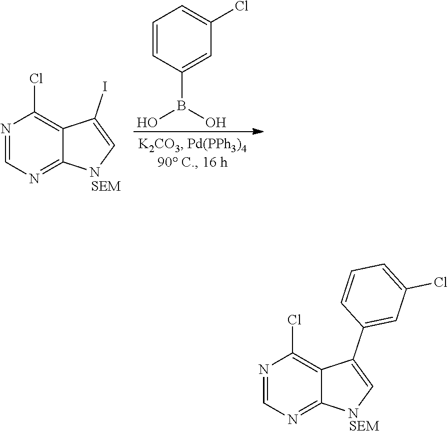

To a solution of 4-chloro-5-iodo-7-((2-(trimethylsilyl)ethoxy)methyl)-7H-pyrrolo[2,3-d]pyrimidine (6.5 g, 15.9 mmol) in dioxane and H₂O (20 mL: 2 mL) were added (3-chlorophenyl) boronic acid (3.0 g, 19.1 mmol), K₂CO₃ (6.56 g, 47.5 mmol) and tetratriphenylphosphine palladium (1.4 g, 1.3 mmol). The mixture was stirred at 80° C. for 16 h under N₂. The mixture was cooled to room temperature and filtered. The filter cake was washed with EA (10 mL×3). The combined layers were washed with brine (15 mL), dried with Na₂SO₄. Filtered and the filtrate was concentrated to obtain the crude product which was purified by flash column chromatography to afford 4-chloro-5-(3-chlorophenyl)-7-((2-(trimethylsilyl)ethoxy)methyl)-7H-pyrrolo[2,3-d]pyrimidine (3.9 g, 62.9%) as a light yellow solid. HPLC/UV purity: 95%; LC-MS (ESI): 393.8 (M+1)+.

### Example 2b Synthesis 5-(3-chlorophenyl)-4-(4-methylpiperazin-1-yl)-7H-pyrrolo[2,3-d]pyrimidine (Compound 47) starting from common intermediate 2

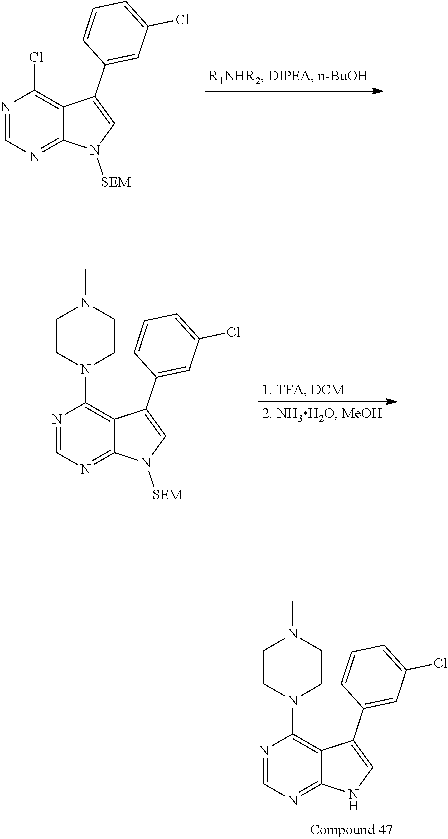 [SMILES: C[Si](C)(C)CCOCn1cc(-c2cccc(Cl)c2)c2c(Cl)ncnc21 ; CN1CCN(c2ncnc3c2c(-c2cccc(Cl)c2)cn3COCC[Si](C)(C)C)CC1 ; CN1CCN(c2ncnc3[nH]cc(-c4cccc(Cl)c4)c23)CC1]

### Step 1

The mixture of 4-chloro-5-(3-chlorophenyl)-7-((2-(trimethylsilyl)ethoxy)methyl)-7H-pyrrolo[2,3-d]pyrimidine (110 mg, 0.28 mmol) and 1-methylpiperazine (84 mg, 0.84 mmol) in n-BuOH (10 mL) was heated at 100° C. for 18 h. The reaction mixture was cooled to room temperature and the solvent was concentrated in vacuo. The residue was diluted with EtOAc (10 mL), washed with water (10 mL×3) and brine (10 mL), dried over Na₂SO₄, and concentrated. The crude product was purified by Prep-TLC to afford 5-(3-chlorophenyl)-4-(4-methylpiperazin-1-yl)-7-((2-(trimethylsilyl)ethoxy)methyl)-7H-pyrrolo[2,3-d]pyrimidine (60 mg, 47%) as an oil. HPLC/UV purity: 90%; LC-MS (ESI): 458.5 [M+H]⁺.

### Step 2

The mixture of 5-(3-chlorophenyl)-4-(4-methylpiperazin-1-yl)-7-((2-(trimethylsilyl)ethoxy) methyl)-7H-pyrrolo[2,3-d]pyrimidine (60 mg, 0.13 mmol) and TFA (2 mL) in DCM (10 mL) was stirred at room temperature for 2 h. The reaction mixture was then concentrated in vacuo to give an intermediate, which was diluted with a mixture of aqueous ammonia (2 mL), DCM (5 mL) and methanol (5 mL). The resulting mixture was stirred at room temperature for an additional 18 h and concentrated in vacuo. The crude product was purified by prep-HPLC to afford 5-(3-chlorophenyl)-4-(4-methylpiperazin-1-yl)-7H-pyrrolo[2,3-d]pyrimidine (18 mg, 48%) as a white solid. HPLC/UV purity: 100%; LC-MS (ESI): 328.1 [M+H]⁺. ¹H NMR (400 MHZ, MeOD-d₄): δ 8.46 (s, 1H), 7.61 (d, J=1.6 Hz, 1H), 7.52-7.46 (m, 3H), 7.39-7.37 (m, 1H), 3.95-4.01 (m, 2H), 3.36-2.97 (m, 6H), 2.86 (s, 3H).

The following compounds were prepared using analogous procedures to Examples 2a and 2b.

| Name | Structure | Characterization |
|---|---|---|
| 5-(3-Chlorophenyl)- 4-(piperidin-1-yl)- 7H-pyrrolo[2,3- d]pyrimidine (Compound 15) | 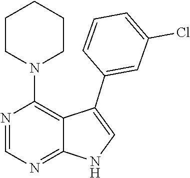 [SMILES: Clc1cccc(-c2c[nH]c3ncnc(N4CCCCC4)c23)c1] | HPLC/UV purity: 100%; LC-MS (ESI): 313.1 [M + H] ⁺. ¹H NMR (400 MHz, DMSO-d₆): δ 12.14 (s, 1H), 8.33 (s, 1H), 7.55 (d, J = 2.2 Hz, 2H), 7.49 (dt, J = 7.8, 1.5 Hz, 1H), 7.44 (t, J = 7.7 Hz, 1H), 7.32 (ddd, J = 7.7, 2.2, 1.3 Hz, 1H), 3.20-3.13 (m, 4H), 1.48-1.40 (m, 2H), 1.39-1.31 (m, 4H). HRMS (ESI-TOF) calculated for C₁₇H₁₈ClN₄ [M + H] ⁺: 313.1220; found 313.1205 |
| 1-(5-(3- Chlorophenyl)-7H- pyrrolo[2,3- d]pyrimidin-4- yl)piperidin-4-ol (Compound 16) | 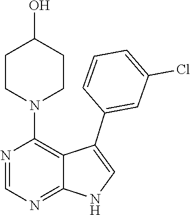 [SMILES: OC1CCN(c2ncnc3[nH]cc(-c4cccc(Cl)c4)c23)CC1] | HPLC/UV purity: 100%; LC-MS (ESI): 329.1 [M + H] ⁺. ¹H NMR (400 MHz, DMSO-d₆): δ 12.40 (s, 1H), 8.37 (s, 1H), 7.61 (d, J = 2.5 Hz, 1H), 7.55 (t, J = 1.7 Hz, 1H), 7.48-7.45 (m, 2H), 7.40-7.32 (m, 1H), 4.07 (br. s, 1H), 3.63-3.52 (m, 3H), 3.02-2.91 (m, 2H), 1.61-1.52 (m, 2H), 1.33-1.20 (m, 2H). HRMS (ESI-TOF) calculated for C₁₇H₁₈ClN₄O [M + H] ⁺: 329.1169; found 329.1172 |
| 5-(3-Chlorophenyl)- N,N-dimethyl-7H- pyrrolo[2,3- d]pyrimidin-4-amine (Compound 17) | 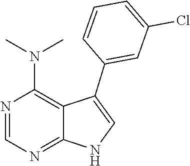 [SMILES: CN(C)c1ncnc2[nH]cc(-c3cccc(Cl)c3)c12] | HPLC/UV purity: 96%; LC-MS (ESI): 273.1 [M + H] ⁺. ¹H NMR (400 MHz, DMSO-d₆): δ 8.27 (s, 1H), 7.57- 7.51 (m, 1H), 7.49 (s, 1H), 7.44-7.40 (m, 2H), 7.35- 7.30 (m, 1H), 5.75 (s, 1H), 2.75 (s, 6H). |
| 5-(3-Chlorophenyl)- N- (cyclopropylmethyl)- 7H-pyrrolo[2,3- d]pyrimidin-4-amine (Compound 18) | 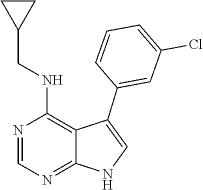 [SMILES: Clc1cccc(-c2c[nH]c3ncnc(NCC4CC4)c23)c1] | HPLC/UV purity: 100%; LC-MS (ESI): 299.1 [M + H] ⁺. ¹H NMR (400 MHz, DMSO-d₆): δ 8.34 (s, 1H), 7.56- 7.52 (m, 2H), 7.50 (d, J = 7.7 Hz, 1H), 7.48-7.41 (m, 2H), 6.68 (d, J = 6.2 Hz, 1H), 3.37 (t, J = 6.3 Hz, 2H), 1.13 (tt, J = 7.6, 4.6 Hz, 1H), 0.48 (dt, J = 6.5, 3.9 Hz, 2H), 0.28 (dt, J = 6.2, 4.3 Hz, 2H). |

### Example 3: Synthesis of 4-(3-(3-Chlorophenyl)-1H-pyrrolo[3,2-c]pyridin-4-yl)morpholine (Compound 22)

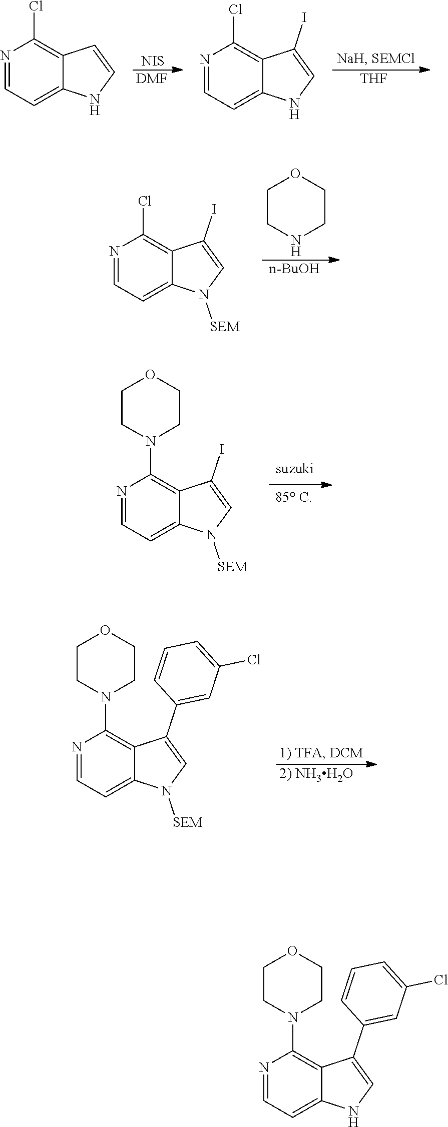

### Step 1

To a solution of 4-chloro-1H-pyrrolo[3,2-c]pyridine (945 mg, 6.22 mmol) in DMF (15 mL) was added NIS (1.4 g, 6.22 mmol) portion wise. The resulting reaction mixture was stirred at room temperature for 18 h. The reaction mixture was quenched with ice water (50 mL), and then extracted with EtOAc (40 mL×3). The organic layer was washed with water (40 mL×3) and brine (40 mL), dried over Na₂SO₄, and concentrated. The crude product was purified by flash column chromatography to afford 4-chloro-3-iodo-1H-pyrrolo[3,2-c]pyridine (1.63 g, 92%) as a white solid. HPLC/UV purity: 90%; LC-MS (ESI): 278.9 [M+H]⁺.

### Step 2

To a solution of 4-chloro-3-iodo-1H-pyrrolo[3,2-c]pyridine (1.45 g, 5.22 mmol) in THF (20 mL) was added NaH (229 mg, 5.74 mmol, 60% in oil) portion wise at 0° C. under N₂. The mixture was stirred at 0° C. for 30 min, and then 2-(trimethylsilyl)ethoxymethyl chloride (953 mg, 5.74 mmol) was added drop wise at 0° C. The resulting mixture was stirred at room temperature for 4 h. The reaction mixture was quenched with ice water (30 mL), and then extracted with EtOAc (20 mL×3). The organic layer was washed with water (20 mL) and brine (20 mL), dried over Na₂SO₄, and concentrated. The crude product was purified by flash column chromatography to afford 4-chloro-3-iodo-1-((2-(trimethyl-silyl)ethoxy)methyl)-1H-pyrrolo[3,2-c]pyridine (1.4 g, 63%) as a yellow solid. HPLC/UV purity: 90%; LC-MS (ESI): 409.1 [M+H]⁺.

### Step 3

In a sealed tube: The mixture of 4-chloro-3-iodo-1-((2-(trimethylsilyl)ethoxy)methyl)-1H-pyrrolo[3,2-c]pyridine (408 mg, 1 mmol) and morpholine (174 mg, 2 mmol) in n-BuOH (10 mL) was heated at 130° C. for 18 h (starting material remaining), then heated at 165° C. for an additional 8 h. The mixture was cooled to room temperature and the solvent was concentrated in vacuo. The residue was diluted with EtOAc (10 mL), washed with water (10 mL×3) and brine (10 mL), dried over Na₂SO₄, and concentrated. The crude product was purified by flash column chromatography to afford 4-(3-iodo-1-((2-(trimethylsilyl)ethoxy)methyl)-1H-pyrrolo[3,2-c]pyridin-4-yl)morpholine (100 mg, 22%) as a solid. HPLC/UV purity: 90%; LC-MS (ESI): 460.2 [M+H]⁺.

### Step 4

The mixture of 4-(3-iodo-1-((2-(trimethylsilyl)ethoxy)methyl)-1H-pyrrolo[3,2-c]pyridin-4-yl)morpholine (100 mg, 0.22 mmol), (3-chlorophenyl) boronic acid (34 mg, 0.22 mmol), Pd(PPh₃)+ (20 mg, 0.1 mmol) and Na₂CO₃ (69 mg, 0.65 mmol) in 1,4-dioxane (10 mL) and H₂O (1 mL) was heated at 85° C. for 18 h under N₂ atmosphere. The reaction mixture was cooled to room temperature and filtered through a pad of celite and concentrated in vacuo. The crude residue was diluted with EtOAc (10 mL), washed by water and brine, dried over Na₂SO₄, and concentrated. The crude product was purified by prep-TLC to afford 4-(3-(3-chlorophenyl)-1-((2-(trimethylsilyl)ethoxy)methyl)-1H-pyrrolo[3,2-c]pyridin-4-yl)morpholine (89 mg, 82%) as a white solid. HPLC/UV purity: 90%; LC-MS (ESI): 444.2 [M+H]⁺.

### Step 5

The mixture of 4-(3-(3-chlorophenyl)-1-((2-(trimethylsilyl)ethoxy)methyl)-1H-pyrrolo[3,2-c]pyridin-4-yl)morpholine (89 mg, 0.12 mmol) and TFA (2 mL) in DCM (2 mL) was stirred at room temperature for 2 h. The reaction mixture was concentrated in vacuo to give an intermediate, which was diluted with a mixture of aqueous ammonia (1 mL), DCM (5 mL) and methanol (5 mL). The resulting mixture was stirred at room temperature for 18 h, then concentrated in vacuo. The crude product was purified by prep-HPLC to afford 4-(3-(3-chlorophenyl)-1H-pyrrolo[3,2-c]pyridin-4-yl)morpholine (45 mg, 74%) as a white solid. HPLC/UV purity: 100%; LC-MS (ESI): 314.1 [M+H]⁺. ¹H NMR (400 MHZ, DMSO-d₆): δ 7.91 (d, J=6.5 Hz, 1H), 7.87 (d, J=2.4 Hz, 1H), 7.65-7.62 (m, 1H), 7.54-7.51 (m, 2H), 7.47 (d, J=6.8 Hz, 1H), 7.46-7.42 (m, 1H), 3.42 (t, J=4.7 Hz, 4H), 3.20 (t, J=4.6 Hz, 4H).

### Example 4: Synthesis of 4-(3-(3-chlorophenyl)-1H-pyrazolo[3,4-d]pyrimidin-4-yl)morpholine (Compound 19)

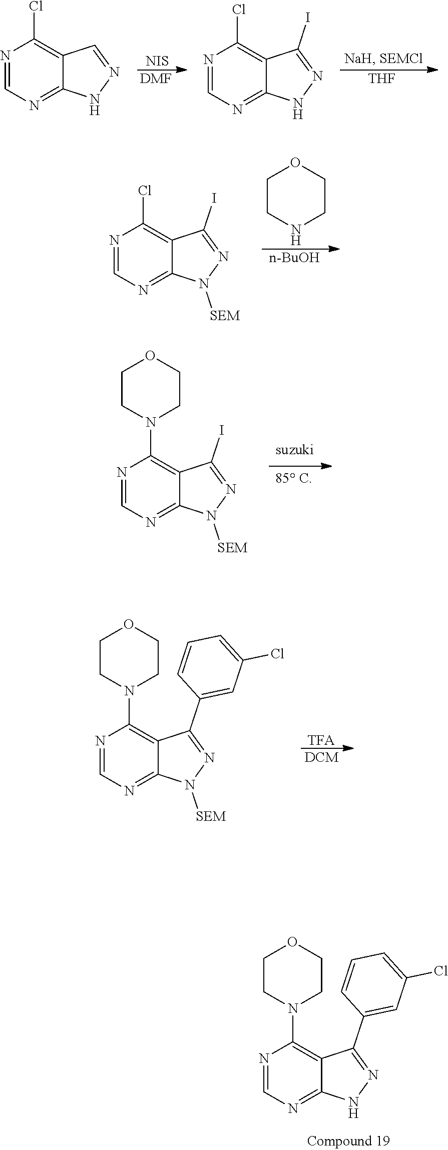

### Step 1

To a solution of 4-chloro-1H-pyrazolo[3,4-d]pyrimidine (1.55 g, 10 mmol) in DMF (20 mL) was added NIS (2.7 g, 12 mmol) portion wise. The resulting reaction mixture was stirred at 50° C. for 18 h. The reaction mixture was cooled to room temperature and poured into ice water (50 mL). A gradual formation of a yellow precipitate was observed. The precipitate was collected and washed with DCM to afford the crude product 4-chloro-3-iodo-1H-pyrazolo[3,4-d]pyrimidine (1.5 g, 54%) which was used to the next step without further purification.

### Step 2

To a solution of 4-chloro-3-iodo-1H-pyrazolo[3,4-d]pyrimidine (1.5 g, 5.4 mmol) in DMF (12 mL) was added NaH (430 mg, 10.7 mmol, 60% in oil) portion wise at 0° C. under N₂. Then the mixture was stirred at 0° C. for 30 min, and 2-(trimethylsilyl)ethoxymethyl chloride (1.17 g, 7 mmol) was added drop wise at 0° C. The resulting mixture was stirred at room temperature overnight. The reaction mixture was quenched with ice water (30 mL), and then extracted with EtOAc (20 mL×3). The organic layer was washed with water (20 mL) and brine (20 mL), dried over Na₂SO₄, and concentrated. The crude product was purified by flash column chromatography to afford 4-chloro-3-iodo-1-((2-(trimethylsilyl)ethoxy)methyl)-1H-pyrazolo[3,4-d]pyrimidine (1.7 g, 77%) as a yellow solid. HPLC/UV purity: 90%; LC-MS (ESI): 411.1 [M+H]⁺.

### Step 3

The mixture of 4-chloro-3-iodo-1-((2-(trimethylsilyl)ethoxy)methyl)-1H-pyrazolo[3,4-d]pyrimidine (400 mg, 1 mmol) and morpholine (264 mg, 3 mmol) in n-BuOH (5 mL) was heated at 100° C. for 18 h. The mixture was cooled to room temperature and the solvent was removed in vacuo. The residue was diluted with EtOAc (10 mL), washed with water (10 mL×3) and brine (10 mL), dried over Na₂SO₄, and concentrated. The crude product was purified by flash column chromatography to afford 4-(3-iodo-1-((2-(trimethylsilyl)ethoxy)methyl)-1H-pyrazolo[3,4-d]pyrimidin-4-yl)morpholine (380 mg, 82%) as a white solid. HPLC/UV purity: 90%; LC-MS (ESI): 462.2 [M+H]⁺.

### Step 4

The mixture of 4-(3-iodo-1-((2-(trimethylsilyl)ethoxy)methyl)-1H-pyrazolo[3,4-d]pyrimidin-4-yl)morpholine (200 mg, 0.43 mmol), (3-chlorophenyl) boronic acid (88 mg, 0.56 mmol), Pd(dppf) Cl2 (25 mg, 0.1 mmol) and Na₂CO₃ (114 mg, 1.1 mmol) in 1,4-dioxane (15 mL) and H₂O (3 mL) was heated at 100° C. for 18 h under a N₂ atmosphere. The reaction mixture was cooled to room temperature, filtered through a pad of celite, and concentrated in vacuo. The crude residue was diluted with EtOAc (10 mL), washed by water and brine, dried over Na₂SO₄, and filtered. The crude product was purified by flash column chromatography to afford 4-(3-(3-chlorophenyl)-1-((2-(trimethylsilyl)ethoxy)methyl)-1H-pyrazolo[3,4-d]pyrimidin-4-yl)morpholine (130 mg, 68%) as a white solid. HPLC/UV purity: 90%; LC-MS (ESI): 446.2 [M+H]⁺.

### Step 5

The mixture of 4-(3-(3-chlorophenyl)-1-((2-(trimethylsilyl)ethoxy)methyl)-1H-pyrazolo[3,4-d]pyrimidin-4-yl)morpholine (130 mg, 0.29 mmol) and TFA (2 mL) in DCM (5 mL) was stirred at room temperature for 2 h, then concentrated in vacuo. The reaction intermediate was diluted with a mixture of aqueous ammonia (1 mL), DCM (5 mL) and methanol (5 mL) and stirred at room temperature for 18 h. The reaction mixture was concentrated in vacuo to give a crude product which was purified by prep-HPLC to afford 4-(3-(3-chlorophenyl)-1H-pyrazolo[3,4-d]pyrimidin-4-yl)morpholine (20 mg, 32%) as a white solid. HPLC/UV purity: 100%; LC-MS (ESI): 316.1 [M+H]⁺. ¹H NMR (400 MHZ, DMSO-d₆): δ 8.45 (s, 1H), 7.73 (t, J=1.7 Hz, 1H), 7.67 (dt, J=7.4, 1.4 Hz, 1H), 7.58 (t, J=7.7 Hz, 1H), 7.53 (dt, J=6.6, 0.9 Hz, 1H), 3.52 (t, J=4.7 Hz, 4H), 3.33 (t, J=4.7 Hz, 4H).

### Example 5: Synthesis of 4-(3-(3-Chlorophenyl)-1H-pyrrolo[2,3-b]pyridin-4-yl)morpholine (Compound 23)

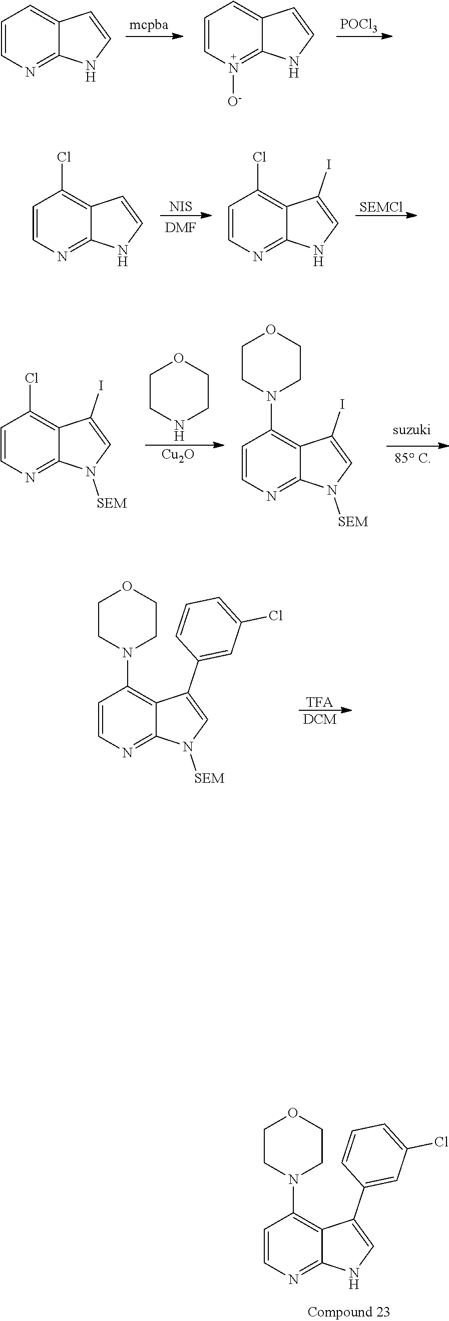

### Step 1

To a solution of 1H-pyrrolo[2,3-b]pyridine (2.4 g, 20 mmol) in DCM (20 mL) was added 3-chloroperoxy benzoic acid (6.1 g, 30 mmol) portion wise at 0° C. The resulting mixture was stirred at room temperature overnight. The precipitate was collected and washed with cold DCM to afford 1H-pyrrolo[2,3-b]pyridine 7-oxide (2.1 g, 91%) as a crude product which was used in the next step without further purification.

### Step 2

The mixture of 1H-pyrrolo[2,3-b]pyridine 7-oxide (1.2 g, 8.96 mmol) in POCl₃ (15 mL) was stirred at 80° C. for 18 h. The reaction mixture was cooled to room temperature and the solvent was removed in vacuo. The residue was diluted with EtOAc (20 mL) and then poured slowly into 1N aq. NaHCO₃ solution (50 mL). The mixture was stirred at room temperature for 30 min. The precipitate was collected by filtration and washed with water (10 mL×3) to afford 4-chloro-1H-pyrrolo[2,3-b]pyridine (1.1 g, 80%) as a crude product which was used to the next step without further purification.

### Step 3

To a solution of 4-chloro-1H-pyrrolo[2,3-b]pyridine (1.1 g, 7.7 mmol) in DMF (10 mL) was added NIS (1.5 g, 8.7 mmol) portion wise. The resulting reaction mixture was stirred at room temperature for 18 h. The reaction mixture was quenched with ice water (50 mL), and then extracted with EtOAc (40 mL×3). The organic layer was washed with water (40 mL×3) and brine (40 mL), dried over Na₂SO₄, and concentrated. The crude product was purified by flash column chromatography to afford 4-chloro-3-iodo-1H-pyrrolo[2,3-b]pyridine (1.2 g, 60%) as a yellow solid. HPLC/UV purity: 90%; LC-MS (ESI): 279.2 [M+H]⁺. ¹H NMR (400 MHZ, DMSO-d₆): δ 12.45 (s, 1H), 8.18 (d, J=9.1 Hz, 1H), 7.81 (s, 1H), 7.19 (d, J=9.4 Hz, 1H).

### Step 4

To a solution of 4-chloro-3-iodo-1H-pyrrolo[2,3-b]pyridine (1.2 g, 4.3 mmol) in THF (15 mL) was added NaH (342 mg, 8.6 mmol, 60% in oil) portion wise at 0° C. under N2. The mixture was stirred at 0° C., for 30 min, and then 2-(trimethylsilyl)ethoxymethyl chloride (867 mg, 5.2 mmol) was added drop wise at 0° C. The resulting mixture was stirred at room temperature overnight. The reaction mixture was quenched with ice water (30 mL), and then extracted with EtOAc (20 mL×3). The organic layer was washed with water (20 mL) and brine (20 mL), dried over Na₂SO₄, and concentrated. The crude product was purified by flash column chromatography to afford 4-chloro-3-iodo-1-((2-(trimethylsilyl)ethoxy)methyl)-1H-pyrrolo[2,3-b]pyridine (830 mg, 47%) as oil. HPLC/UV purity: 90%; LC-MS (ESI): 409.1 [M+H]⁺. ¹H NMR (400 MHZ, DMSO-d₆): δ 8.25 (d, J=5.1 Hz, 1H), 8.01 (s, 1H), 7.27 (d, J=5.1 Hz, 1H), 5.59 (s, 2H), 3.49 (t, J=8.1 Hz, 2H), 0.80 (t, J=8.1 Hz, 2H), —0.12 (s, 9H).

### Step 5

The mixture of 4-chloro-3-iodo-1-((2-(trimethylsilyl)ethoxy)methyl)-1H-pyrrolo[2,3-b]pyridine (840 mg, 2 mmol), cuprous oxide (328 mg, 2.3 mmol) and morpholine (5 g, 69 mmol) in 1,4-dioxane (2.1 mL) and ethylene glycol (8 mL) was heated at 130° C. for 18 h under a N₂ atmosphere. The reaction mixture was cooled to room temperature and filtered through a pad of celite and concentrated. The crude residue was diluted with EtOAc (10 mL), washed by water and brine, dried over Na₂SO₄, and concentrated in vacuo. The crude product was purified by flash column chromatography to afford 4-(3-iodo-1-((2-(trimethylsilyl)ethoxy)methyl)-1H-pyrrolo[2,3-b]pyridin-4-yl)morpholine (80 mg, 9%) as a solid. HPLC/UV purity: 90%; LC-MS (ESI): 460.2 [M+H]⁺.

### Step 6

The mixture of 4-(3-iodo-1-((2-(trimethylsilyl)ethoxy)methyl)-1H-pyrrolo[2,3-b]pyridin-4-yl)morpholine (80 mg, 0.17 mmol), (3-chlorophenyl) boronic acid (33 mg, 0.21 mmol), Pd(PPh₃)₄ (20 mg, 0.1 mmol) and Na₂CO₃ (37 mg, 0.34 mmol) in 1,4-dioxane (20 mL) and H₂O (4 mL) was heated at 100° C. for 18 h under a N₂ atmosphere. The reaction mixture was cooled to room temperature and filtered through a pad of celite and concentrated. The crude residue was diluted with EtOAc (10 mL), washed by water and brine, dried over Na₂SO₄, and concentrated in vacuo. The crude product was purified by prep-TLC to afford 4-(3-(3-chlorophenyl)-1-((2-(trimethylsilyl)ethoxy)methyl)-1H-pyrrolo[2,3-b]pyridin-4-yl)morpholine (56 mg, 75%) as a solid. HPLC/UV purity: 90%; LC-MS (ESI): 444.2 [M+H]⁺.

### Step 7

The mixture of 4-(3-(3-chlorophenyl)-1-((2-(trimethylsilyl)ethoxy)methyl)-1H-pyrrolo[2,3-b]pyridin-4-yl)morpholine (56 mg, 0.13 mmol) and TFA (2 mL) in DCM (5 mL) was stirred at room temperature for 2 h. The reaction mixture was concentrated in vacuo to give an intermediate, which was diluted with a mixture of aqueous ammonia (1 mL), DCM (5 mL) and methanol (5 mL). The resulting mixture was stirred at room temperature for 18 h, then concentrated in vacuo. The crude product was purified by prep-HPLC to afford 4-(3-(3-chlorophenyl)-1H-pyrrolo[2,3-b]pyridin-4-yl)morpholine (25 mg, 41%) as a white solid. HPLC/UV purity: 100%; LC-MS (ESI): 314.2 [M+H]⁺. ¹H NMR (400 MHZ, DMSO-d₆): δ 11.94 (s, 1H), 8.13 (d, J=5.4 Hz, 1H), 7.67 (t, J=2.1 Hz, 1H), 7.59-7.54 (m, 2H), 7.43 (t, J=7.8 Hz, 1H), 7.35-7.28 (m, 1H), 6.68 (d, J=5.4 Hz, 1H), 3.45 (t, J=4.7 Hz, 4H), 2.85 (t, J=4.5 Hz, 4H). HRMS (ESI-TOF) calculated for C17H17ClN3O [M+H]⁺: 314.1060; found 314.1054.

### Example 6: Synthesis of 4-(5-(3-Chlorophenyl) thieno[2,3-d]pyrimidin-4-yl)morpholine (Compound 20)

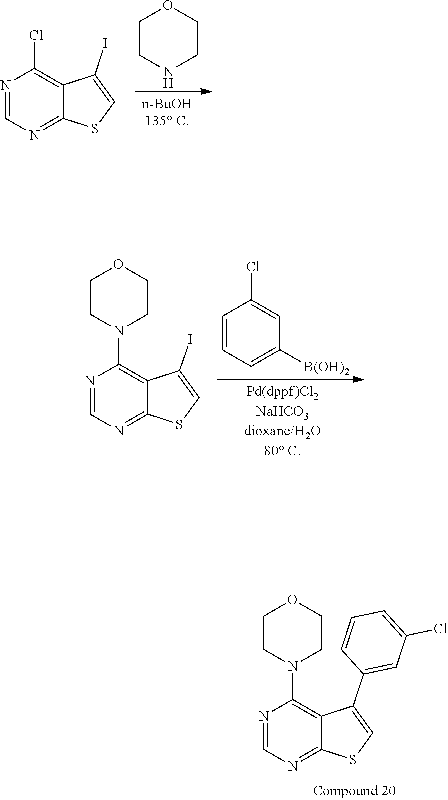

### Step 1

The mixture of 4-chloro-5-iodothieno[2,3-d]pyrimidine (89 mg, 0.3 mmol) and morpholine (52 mg, 0.6 mmol) in n-BuOH (5 mL) was heated at 100° C. for 3 h. The mixture was cooled to room temperature and the solvent was removed in vacuo. The residue was diluted with EtOAc (10 mL), washed with water (10 mL×3) and brine (10 mL), dried over Na₂SO₄, and filtered. The filtrate was concentrated in vacuo to give 4-(5-iodothieno[2,3-d]pyrimidin-4-yl)morpholine as a crude product (100 mg, 96%) which was used to the next step without purification. HPLC/UV purity: 90%; LC-MS (ESI): 348.2 [M+H]⁺. ¹H NMR (400 MHZ, DMSO-d₆): δ 8.61 (s, 1H), 8.14 (s, 1H), 3.84 (t, J=4.6 Hz, 4H), 3.45 (t, J=4.6 Hz, 4H).

### Step 2

The mixture of 4-(5-iodothieno[2,3-d]pyrimidin-4-yl)morpholine (69 mg, 0.2 mmol), (3-chlorophenyl) boronic acid (47 mg, 0.3 mmol), Pd(dppf) Cl2 (15 mg, 0.02 mmol) and NaHCO₃ (50 mg, 0.6 mmol) in 1,4-dioxane (10 mL) and H₂O (1 mL) was heated at 80° C. for 18 h under a N₂ atmosphere. The reaction mixture was cooled to room temperature and filtered through a pad of celite and concentrated in vacuo. The crude residue was diluted with EtOAc (10 mL), washed by water and brine, dried over Na₂SO₄, and concentrated. The crude product was purified by prep-HPLC to afford 4-(5-(3-chlorophenyl) thieno[2,3-d]pyrimidin-4-yl)morpholine (60 mg, 91%) as a solid. HPLC/UV purity: 100%; LC-MS (ESI): 332.2 [M+H]⁺. ¹H NMR (400 MHZ, MeOD-d₄): δ 8.61 (s, 1H), 7.68 (s, 1H), 7.53-7.45 (m, 4H), 3.38-3.35 (m, 4H), 3.32-3.29 (m, 4H). ¹H NMR (400 MHZ, DMSO-d₆): δ 8.63 (s, 1H), 7.86 (s, 1H), 7.58-7.45 (m, 4H), 3.24 (t, J=4.5 Hz, 4H), 3.10 (t, J=4.1 Hz, 4H).

### Example 7: Synthesis of 5-(3-Chlorophenyl)-4-morpholinofuro[2,3-d]pyrimidine (Compound 21)

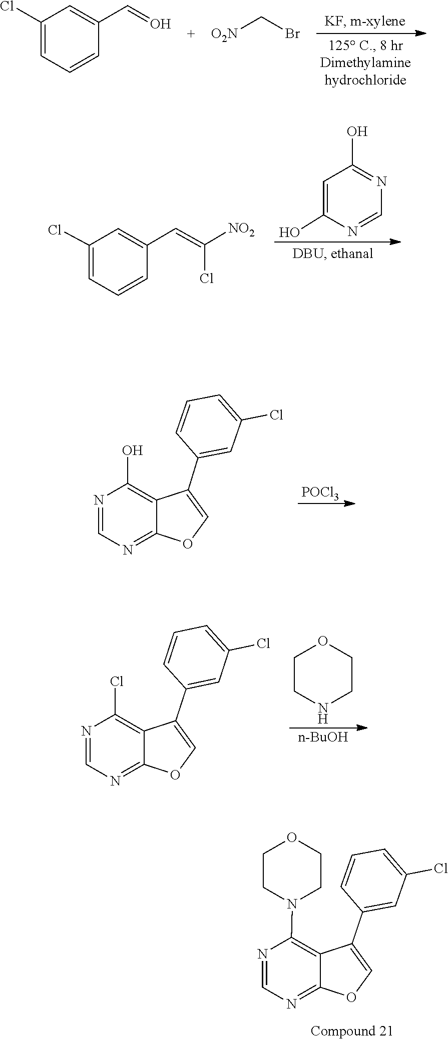

### Step 1

The mixture of 3-chlorobenzaldehyde (2 g, 14.2 mmol), bromo (nitro) methane (4 g, 28.4 mmol), KF (124 mg, 2.1 mmol) and dimethylamine hydrochloride (10 g, 128 mmol) in m-xylene was refluxed for 24 h while water was azeotropically removed using a Dean-Stark apparatus. The reaction mixture was cooled to room temperature and concentrated in vacuo. The crude residue was diluted with EtOAc (10 mL), washed by water and brine, dried over Na₂SO₄, and concentrated in vacuo. The crude product was purified by flash column chromatography to afford (Z)-1-chloro-3-(2-chloro-2-nitrovinyl) benzene (1.7 g, 55%) as a yellow solid. HPLC/UV purity: 90%; LC-MS (ESI): 218.2 [M+H]⁺. ¹H NMR (400 MHZ, CDCl₃): δ 8.30 (s, 1H), 7.85 (t, J=1.9 Hz, 1H), 7.70 (dt, J=7.5, 1.7 Hz, 1H), 7.49 (dt, J=8.1, 1.7 Hz, 1H), 7.44 (t, J=7.8 Hz, 1H).

### Step 2

The mixture of (Z)-1-chloro-3-(2-chloro-2-nitrovinyl) benzene (700 mg, 3.2 mmol) and pyrimidine-4,6-diol (396 mg, 3.53 mmol) in dry ethanol (20 mL) was heated at 60° C. for 10 min until pyrimidine-4,6-diol dissolved completely, and DBU (974 mg, 6.4 mmol) was added drop wise. The resulting mixture was stirred at 80° C. for 18 h, then cooled to room temperature and concentrated in vacuo. The crude residue was diluted with DCM (30 mL), washed by water and brine, dried over Na₂SO₄, and concentrated. The crude product was purified by flash column chromatography to afford 5-(3-chlorophenyl)furo[2,3-d]pyrimidin-4-ol (43 mg, 6%) as a brown solid. HPLC/UV purity: 90%; LC-MS (ESI): 247.1 [M+H]⁺. ¹H NMR (400 MHZ, DMSO-d₆): δ 12.75 (br, s, 1H), 8.38 (s, 1H), 8.18 (s, 1H), 7.97 (d, J=7.7 Hz, 1H), 7.49-7.36 (m, 3H).

### Step 3

The mixture of 5-(3-chlorophenyl)furo[2,3-d]pyrimidin-4-ol (43 mg, 0.17 mmol) in POCl₃ (5 mL) was stirred at 100° C. for 1 hr. The reaction mixture was cooled to room temperature and the solvent was removed in vacuo. The residue was diluted with EtOAc (10 mL) and then slowly poured into cold 1N aq. NaHCO₃ solution (20 mL). The mixture was stirred at room temperature for 30 min. The organic layer was washed by water and brine, dried over Na₂SO₄, and concentrated in vacuo. The crude product was purified by Prep-TLC to afford 4-chloro-5-(3-chlorophenyl)furo[2,3-d]pyrimidine (25 mg, 55%) as a white solid. HPLC/UV purity: 90%; LC-MS (ESI): 265.1 [M+H]⁺. ¹H NMR (400 MHz, DMSO-d₆): δ 8.90 (s, 1H), 8.55 (s, 1H), 7.71-7.68 (m, 1H), 7.62-7.52 (m, 3H).

### Step 4

The mixture of 4-chloro-5-(3-chlorophenyl)furo[2,3-d]pyrimidine (25 mg, 0.09 mmol) and morpholine (16 mg, 0.18 mmol) in n-BuOH (3 mL) was heated at 135° C. for 3 h. The mixture was cooled to room temperature and the solvent was removed in vacuo. The residue was diluted with EtOAc (10 mL), washed with water (10 mL×3) and brine (10 mL), dried over Na₂SO₄, and concentrated in vacuo. The crude product was purified by Prep-TLC to afford 5-(3-chlorophenyl)-4-morpholinofuro[2,3-d]pyrimidine (27 mg, 96%) as a white solid. HPLC/UV purity: 100%; LC-MS (ESI): 316.1 [M+H]⁺. ¹H NMR (400 MHZ, DMSO-d₆): δ 8.51 (s, 1H), 8.27 (s, 1H), 7.62 (t, J=1.8 Hz, 1H), 7.59-7.46 (m, 3H), 3.48 (t, J=4.7 Hz, 4H), 3.22 (t, J=4.7 Hz, 4H).

### Example 8: Synthesis of 5-(3-Chlorophenyl)-4-(4-methoxypiperidin-1-yl)-7H-pyrrolo[2,3-d]pyrimidine (Compound 110)

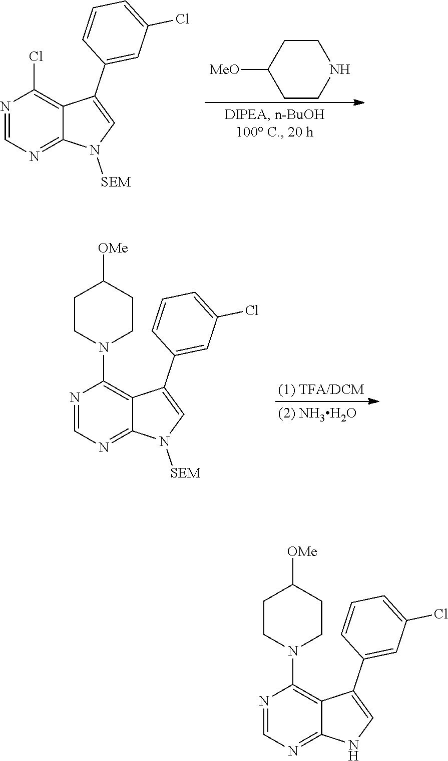 [SMILES: C[Si](C)(C)CCOCn1cc(-c2cccc(Cl)c2)c2c(Cl)ncnc21 ; COC1CCNCC1 ; COC1CCN(c2ncnc3c2c(-c2cccc(Cl)c2)cn3COCC[Si](C)(C)C)CC1 ; COC1CCN(c2ncnc3[nH]cc(-c4cccc(Cl)c4)c23)CC1]

### 5-(3-Chlorophenyl)-4-(4-methoxypiperidin-1-yl)-7-((2-(trimethylsilyl)ethoxy)methyl)-7H-pyrrolo[2,3-d]pyrimidine

To a solution of 4-chloro-5-(3-chlorophenyl)-7-((2-(trimethylsilyl)ethoxy)methyl)-7H-pyrrolo[2,3-d]pyrimidine (300.0 mg, 0.8 mmol, 1.0 equiv) in BuOH (10 mL) were added 4-methoxypiperidine (131.7 mg, 1.1 mmol, 1.5 equiv) and N-ethyl-N-isopropylpropan-2-amine (296.0 mg, 2.3 mmol, 3.0 equiv). The mixture was stirred at 100° C. for overnight. The mixture was concentrated to obtain the crude product which was purified by flash column chromatography (0˜25% EtOAc/Petroether) to provide 5-(3-chlorophenyl)-4-(4-methoxypiperidin-1-yl)-7-((2-(trimethylsilyl)ethoxy)methyl)-7H-pyrrolo[2,3-d]pyrimidine 2 (210.0 mg, 58.3%) as a yellow solid. LC-MS (ESI): 473.1 [M+H]⁺, purity >98% (214, 254 nm); ¹H NMR (400 MHZ, CDCl₃) δ 8.53 (s, 1H), 7.57 (s, 1H), 7.44-7.38 (m, 2H), 7.34-7.31 (m, 1H), 7.31 (s, 1H), 5.67 (s, 2H), 3.61-3.31 (m, 4H), 3.31-3.36 (m, 4H), 3.08 (t, J=9.6 Hz, 2H), 1.80-1.75 (m, 2H), 1.50-1.46 (m, 2H), 0.98 (t, J=9.6 Hz, 2H), 0.00 (s, 9H).

### 5-(3-Chlorophenyl)-4-(4-methoxypiperidin-1-yl)-7H-pyrrolo[2,3-d]pyrimidine

To a solution of 5-(3-chlorophenyl)-4-(4-methoxypiperidin-1-yl)-7-((2-(trimethylsilyl)ethoxy)methyl)-7H-pyrrolo[2,3-d]pyrimidine (2) (205 mg, 0.4 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to give the crude product which was purified by prep-HPLC to afford the compound 5-(3-chlorophenyl)-4-(4-methoxypiperidin-1-yl)-7H-pyrrolo[2,3-d]pyrimidine (120.2 mg, 80.9%) as a white solid. LC-MS: 342.8 [M+H]⁺, purity >98% (214, 254 nm); ¹H NMR (400 MHz, DMSO-d₆) δ 12.16 (s, 1H), 8.35 (s, 1H), 7.57 (dd, J=4.8, 3.6 Hz, 1H), 7.51-7.43 (m, 2H), 7.34 (t, J=3.8 Hz, 1H), 3.53-3.49 (m, 2H), 3.34-3.23 (m, 1H), 3.18 (s, 3H), 2.95-2.88 (m, 2H), 1.69-1.66 (m, 2H), 1.34-1.27 (m, 2H).

### Example 9: Synthesis of 1-(5-(3-chlorophenyl)-7H-pyrrolo[2,3-d]pyrimidin-4-yl)-4-methylpiperidin-4-amine (Compound 102)

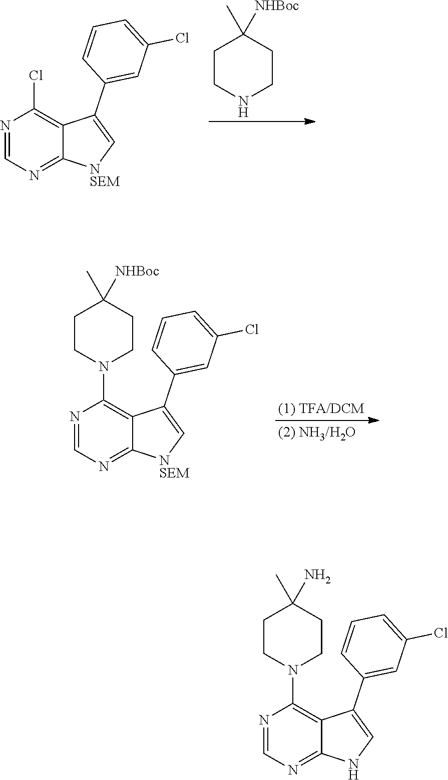

To a solution of tert-butyl (1-(5-(3-chlorophenyl)-7-((2-(trimethylsilyl)ethoxy) methyl)-7H-pyrrolo[2,3-d]pyrimidin-4-yl)-4-methylpiperidin-4-yl) carbamate (218 mg, 0.38 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product and purified by prep-HPLC to afford the compound 5-(3-chlorophenyl)-4-(4-methoxypiperidin-1-yl)-7H-pyrrolo[2,3-d]pyrimidine (106.1 mg, 81.6%) as a white solid. LC-MS: 341.8 [M+H]⁺, purity >98% (214, 254 nm); ¹H NMR (400 MHZ, DMSO-d₆) δ 12.24 (s, 1H), 8.37 (s, 1H), 7.84 (s, 2H), 7.60 (d, J=8.4 Hz, 2H), 7.51-7.45 (m, 2H), 7.35 (t, J=7.2 Hz, 1H), 3.53-3.49 (m, 2H), 3.17-3.11 (m, 2H), 1.63-1.59 (m, 2H), 1.50-1.46 (m, 2H), 1.23 (s, 3H).

### Example 10: Synthesis of 1-(5-(3-chlorophenyl)-7H-pyrrolo[2,3-d]pyrimidin-4-yl)-4-methylpiperidin-4-ol (Compound 103)

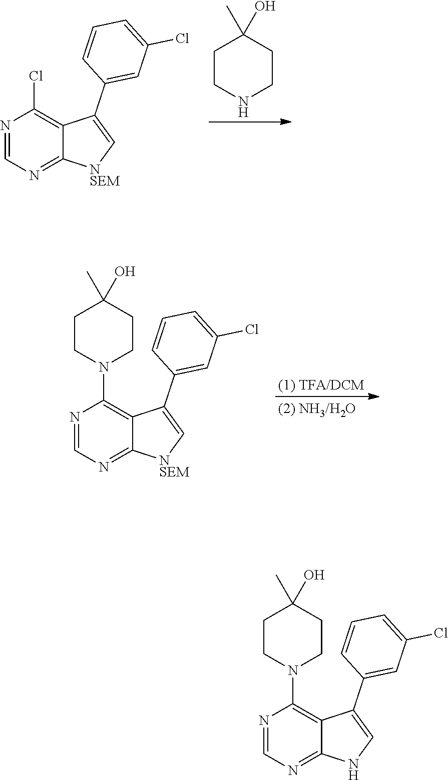

To a solution of 5-(3-chlorophenyl)-4-(4-methoxypiperidin-1-yl)-7-((2-(trimethylsilyl)ethoxy)methyl)-7H-pyrrolo[2,3-d]pyrimidine (2) (163 mg, 0.34 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to give the crude product which was purified by prep-HPLC to afford the compound 5-(3-chlorophenyl)-4-(4-methoxypiperidin-1-yl)-7H-pyrrolo[2,3-d]pyrimidine (75.3 mg, 63.8%) as a white solid. LC-MS: 342.8 [M+H]⁺, purity >98% (214, 254 nm); ¹H NMR (400 MHZ, DMSO-d₆) δ 12.12 (s, 1H), 8.32 (s, 1H), 7.55 (d, J=2.0 Hz, 2H), 7.50-7.42 (m, 2H), 7.32 (d, J=8.0 Hz, 1H), 4.21 (s, 1H), 3.43-3.40 (m, 2H), 3.11-3.04 (m, 2H), 1.38-1.27 (m, 4H), 1.06 (s, 3H).

### Example 11: Synthesis of 1-((5-(3-chlorophenyl)-7H-pyrrolo[2,3-d]pyrimidin-4-yl)amino)propan-2-ol (Compound 104)

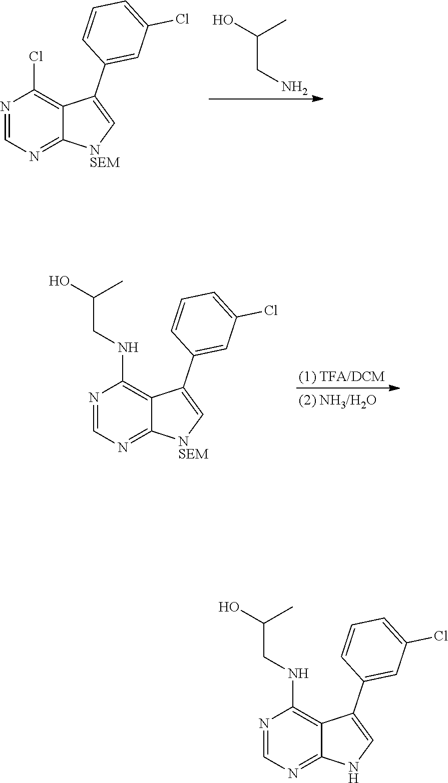

To a solution of 1-((5-(3-chlorophenyl)-7-((2-(trimethylsilyl)ethoxy)methyl)-7H-pyrrolo[2,3-d]pyrimidin-4-yl)amino)propan-2-ol (124 mg, 0.29 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 1-((5-(3-chlorophenyl)-7H-pyrrolo[2,3-d]pyrimidin-4-yl)amino)propan-2-ol (43.8 mg, 50.5%) as a white solid. LC-MS: 302.8 [M+H]⁺, purity >98% (214, 254 nm); ¹H NMR (400 MHZ, DMSO-d₆) δ 11.90 (s, 1H), 8.18 (s, 1H), 8.74 (s, 1H), 7.48 (t, J=7.6 Hz, 2H), 7.39 (d, J=7.2 Hz, 1H), 7.33 (s, 1H), 5.59-5.56 (m, 1H), 4.81 (d, J=4.0 Hz, 1H), 3.80-3.78 (m, 1H), 3.60-3.54 (m, 2H), 3.28-3.21 (m, 1H), 1.08 (d, J=6.0 Hz, 3H).

### Example 12: Synthesis of N-(1-(5-(3-chlorophenyl)-7H-pyrrolo[2,3-d]pyrimidin-4-yl)piperidin-4-yl)acetamide (Compound 105)

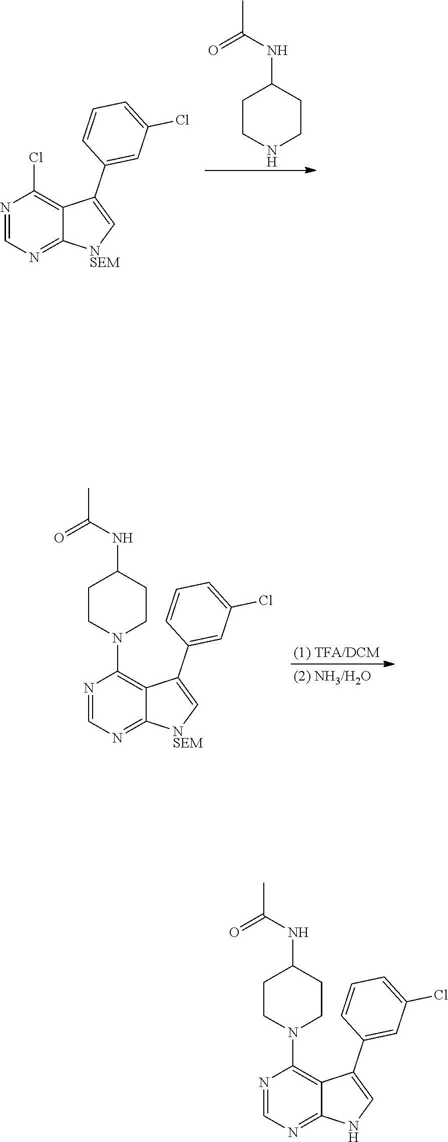

To a solution of N-(1-(5-(3-chlorophenyl)-7-((2-(trimethylsilyl)ethoxy)methyl)-7H-pyrrolo[2,3-d]pyrimidin-4-yl)piperidin-4-yl)acetamide (156 mg, 0.31 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound N-(1-(5-(3-chlorophenyl)-7H-pyrrolo[2,3-d]pyrimidin-4-yl)piperidin-4-yl)acetamide (58.8 mg, 51.1%) as a white solid. LC-MS: 369.8 [M+H]⁺, purity >98% (214, 254 nm); ¹H NMR (400 MHZ, DMSO-d₆) δ 12.18 (s, 1H), 8.35 (s, 1H), 7.76 (d, J=7.2 Hz, 1H), 7.58 (s, 2H), 7.50-7.43 (m, 2H), 7.34 (d, J=7.6 Hz, 1H), 3.69-3.66 (m, 2H), 3.62-3.60 (m, 1H), 2.85-2.79 (m, 2H), 1.76 (s, 3H), 1.59-1.55 (m, 2H), 1.26-1.24 (m, 2H).

### Example 13: Synthesis of 1-(5-(3-chlorophenyl)-7H-pyrrolo[2,3-d]pyrimidin-4-yl)-N-methylpiperidin-4-amine (Compound 106)

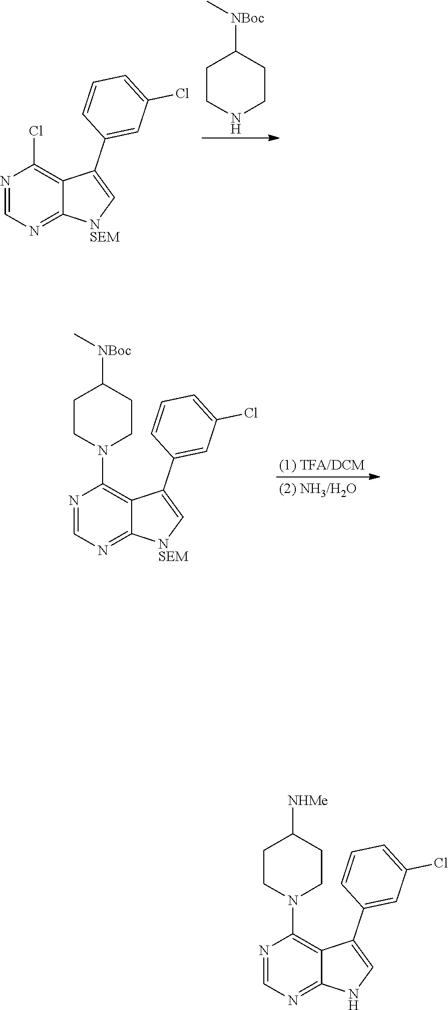

To a solution of tert-butyl (1-(5-(3-chlorophenyl)-7-((2-(trimethylsilyl)ethoxy) methyl)-7H-pyrrolo[2,3-d]pyrimidin-4-yl)piperidin-4-yl)(methyl) carbamate (152 mg, 0.27 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-4-(4-methoxy piperidin-1-yl)-7H-pyrrolo[2,3-d]pyrimidine (66.5 mg, 73.3%) as a white solid. LC-MS: 341.8 [M+H]⁺, purity >98% (214, 254 nm); ¹H NMR (400 MHZ, DMSO-d₆) δ 12.13 (s, 1H), 8.33 (s, 1H), 7.56 (s, 2H), 7.49-7.42 (m, 2H), 7.33 (d, J=7.6 Hz, 1H), 3.66 (d, J=12.8 Hz, 2H), 2.76 (t, J=11.8 Hz, 2H), 2.36-2.32 (m, 1H), 2.20 (s, 3H), 1.60 (d, J=11.2 Hz, 2H), 1.16-1.07 (m, 2H).

### Example 14: Synthesis of 1-(5-(3-chlorophenyl)-7H-pyrrolo[2,3-d]pyrimidin-4-yl)-N,N-dimethylpiperidin-4-amine (Compound 107)

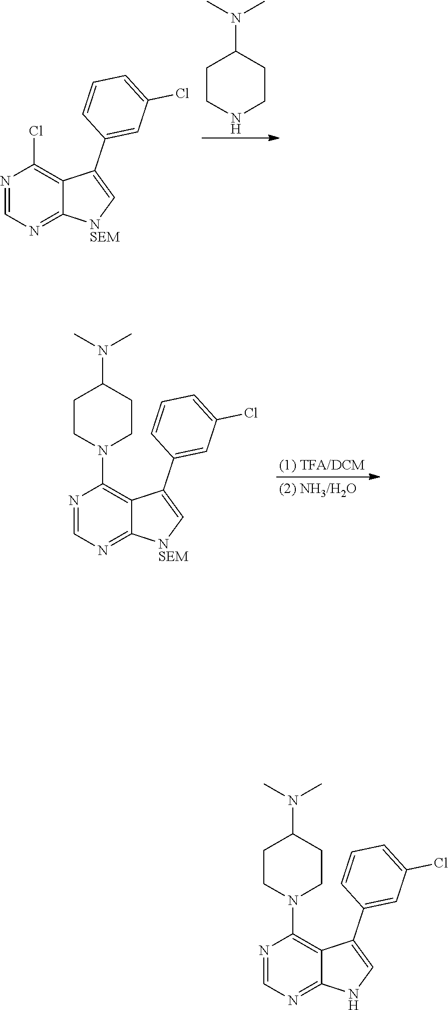

To a solution of 1-(5-(3-chlorophenyl)-7-((2-(trimethylsilyl)ethoxy)methyl)-7H-pyrrolo[2,3-d]pyrimidin-4-yl)-N,N-dimethylpiperidin-4-amine (93 mg, 0.19 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-4-(4-methoxypiperidin-1-yl)-7H-pyrrolo[2,3-d]pyrimidine (22.1 mg, 32.5%) as a white solid. LC-MS: 355.9 [M+H]⁺, purity >98% (214, 254 nm); ¹H NMR (400 MHZ, DMSO-d₆) δ 8.40 (s, 1H), 7.61-7.49 (m, 4H), 7.41 (d, J=7.6 Hz, 1H), 3.83-3.80 (m, 2H), 2.71 (t, J=12.0 Hz, 2H), 2.24-2.19 (m, 1H), 2.17 (s, 6H), 1.59 (d, J=11.2 Hz, 2H), 1.35-1.29 (m, 2H).

### Example 15: Synthesis of 2-((5-(3-chlorophenyl)-7H-pyrrolo[2,3-d]pyrimidin-4-yl)(methyl)amino)ethanol (Compound 108)

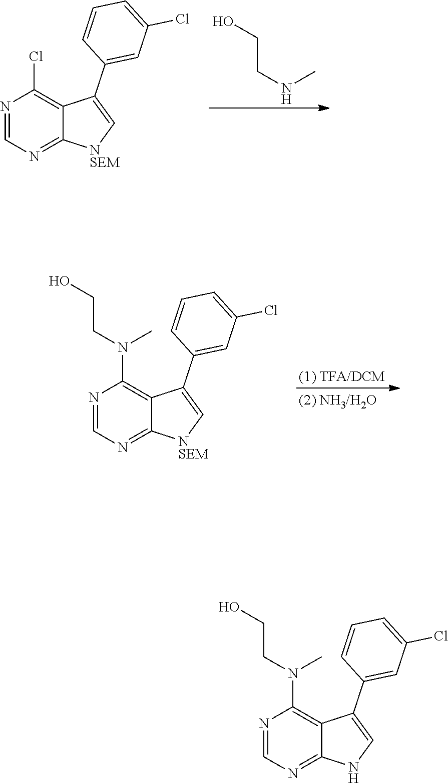

To a solution of 2-((5-(3-chlorophenyl)-7-((2-(trimethylsilyl)ethoxy)methyl)-7H-pyrrolo[2,3-d]pyrimidin-4-yl)(methyl)amino)ethanol (141 mg, 0.33 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-4-(4-methoxypiperidin-1-yl)-7H-pyrrolo[2,3-d]pyrimidine (49.2 mg, 49.9%) as a white solid. LC-MS: 302.7 [M+H]⁺, purity >98% (214, 254 nm); ¹H NMR (400 MHZ, DMSO-d₆) δ 12.03 (s, 1H), 8.23 (s, 1H), 7.54 (s, 1H), 7.46-7.39 (m, 3H), 7.32 (d, J=7.6 Hz, 1H), 4.68-4.65 (m, 1H), 3.51-3.39 (m, 4H), 2.74 (s, 3H).

### Example 16: Synthesis of 2-((5-(3-chlorophenyl)-7H-pyrrolo[2,3-d]pyrimidin-4-yl)(methyl)amino)ethanol (Compound 109)

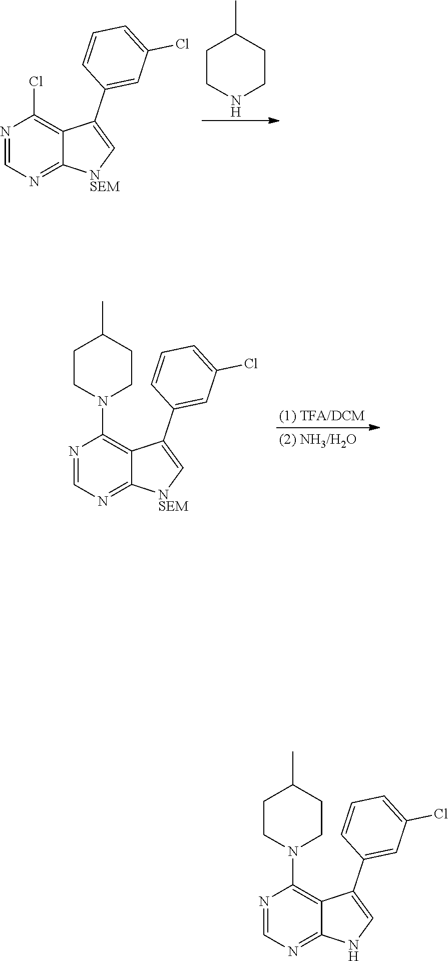

To a solution of 5-(3-chlorophenyl)-4-(4-methylcyclohexyl)-7-((2-(trimethylsilyl)ethoxy)methyl)-7H-pyrrolo[2,3-d]pyrimidine (136 mg, 0.30 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-4-(4-methylcyclohexyl)-7H-pyrrolo[2,3-d]pyrimidine (57.7 mg, 62.6%) as a white solid. LC-MS: 326.8 [M+H]⁺, purity >98% (214, 254 nm); ¹H NMR (400 MHz, DMSO-d₆) δ 12.01 (s, 1H), 8.34 (s, 1H), 7.56 (d, J=4.4 Hz, 2H), 7.51 (d, J=7.6 Hz, 1H), 7.44 (t, J=8.0 Hz, 1H), 7.33 (d, J=7.6 Hz, 1H), 3.72 (d, J=12.8 Hz, 2H), 2.63 (t, J=12.0 Hz, 2H), 1.41 (d, J=10.4 Hz, 3H), 1.05-0.97 (m, 2H), 0.84 (d, J=6.0 Hz, 3H).

### Example 17: Synthesis of 2-((5-(3-chlorophenyl)-7H-pyrrolo[2,3-d]pyrimidin-4-yl)(ethyl)amino)ethanol (Compound 114)

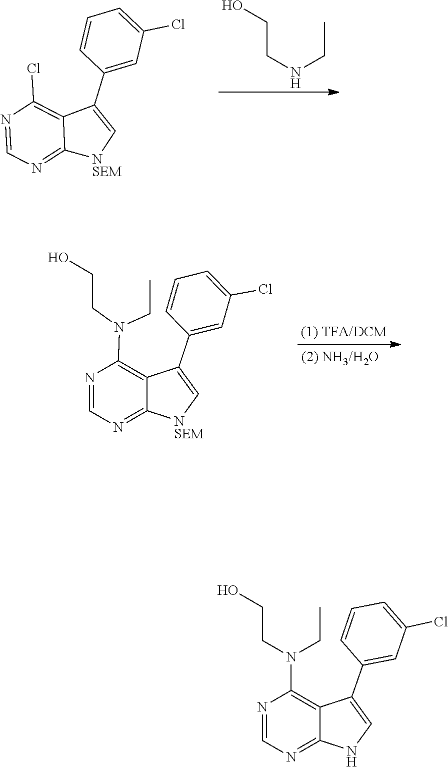

To a solution of 2-((5-(3-chlorophenyl)-7-((2-(trimethylsilyl)ethoxy)methyl)-7H-pyrrolo[2,3-d]pyrimidin-4-yl)(ethyl)amino)ethanol (128 mg, 0.29 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 2-((5-(3-chlorophenyl)-7H-pyrrolo[2,3-d]pyrimidin-4-yl)(ethyl)amino)ethanol (46.2 mg, 50.9%) as a white solid. LC-MS: 316.8 [M+H]⁺, purity >98% (214, 254 nm); ¹H NMR (400 MHZ, DMSO-d₆) δ 12.01 (s, 1H), 8.23 (s, 1H), 7.58 (s, 1H), 7.57-7.51 (m, 2H), 7.43-7.39 (m, 1H), 7.32-7.30 (m, 1H), 4.54 (t, J=5.2 Hz, 1H), 3.46-3.42 (m, 2H), 3.35-3.32 (m, 2H), 3.24 (q, J=6.8 Hz, 2H), 0.89 (t, J=6.8 Hz, 3H).

### Example 18: Synthesis of 5-(3-chlorophenyl)-N-methyl-N-propyl-7H-pyrrolo[2,3-d]pyrimidin-4-amine (Compound 115)

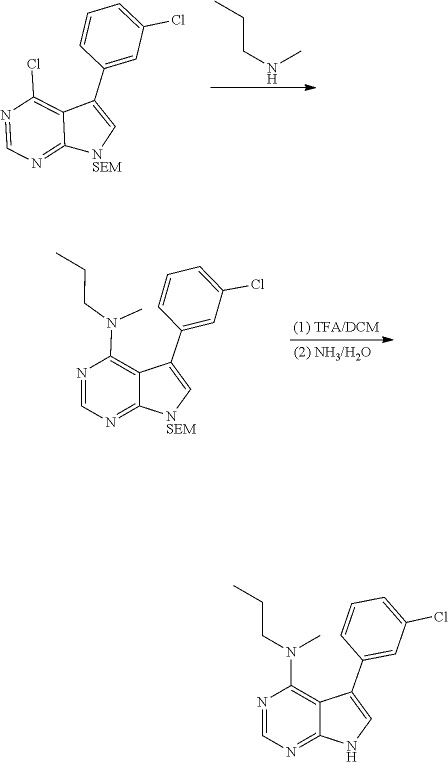

To a solution of 5-(3-chlorophenyl)-N-methyl-N-propyl-7-((2-(trimethylsilyl)ethoxy)methyl)-7H-pyrrolo[2,3-d]pyrimidin-4-amine (175 mg, 0.41 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-N-methyl-N-propyl-7H-pyrrolo[2,3-d]pyrimidin-4-amine (78.5 mg, 64.3%) as a white solid. LC-MS: 300.8 [M+H]⁺, purity >98% (214, 254 nm); ¹H NMR (400 MHZ, DMSO-d₆) δ 12.06 (s, 1H), 8.26 (s, 1H), 7.50 (s, 1H), 7.48 (s, 1H), 7.45-7.40 (m, 2H), 7.36-7.32 (m, 1H), 3.23 (t, J=7.2 Hz, 2H), 2.69 (s, 3H), 1.52-1.42 (m, 2H), 0.68 (t, J=7.2 Hz, 3H).

### Example 19: Synthesis of N-butyl-5-(3-chlorophenyl)-7H-pyrrolo[2,3-d]pyrimidin-4-amine (Compound 116)

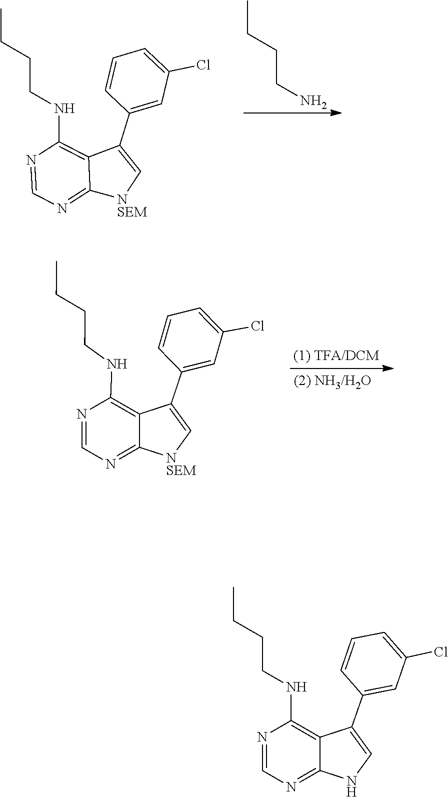

To a solution of N-butyl-5-(3-chlorophenyl)-7-((2-(trimethylsilyl)ethoxy)methyl)-7H-pyrrolo[2,3-d]pyrimidin-4-amine (107 mg, 0.25 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound N-butyl-5-(3-chlorophenyl)-7H-pyrrolo[2,3-d]pyrimidin-4-amine (35.7 mg, 47.9%) as a white solid. LC-MS: 300.8 [M+H]⁺, purity >98% (214, 254 nm); ¹H NMR (400 MHZ, DMSO-d₆) δ 11.89 (s, 1H), 8.19 (s, 1H), 7.50-7.38 (m, 4H), 7.32 (s, 1H), 5.46 (t, J=5.6 Hz, 1H), 3.46-3.41 (m, 2H), 1.53-1.49 (m, 2H), 1.36-1.30 (m, 2H), 0.89 (t, J=7.2 Hz, 3H).

### Example 20: Synthesis of 3-((5-(3-chlorophenyl)-7H-pyrrolo[2,3-d]pyrimidin-4-yl)amino)-2-methylpropan-1-ol (Compound 117)

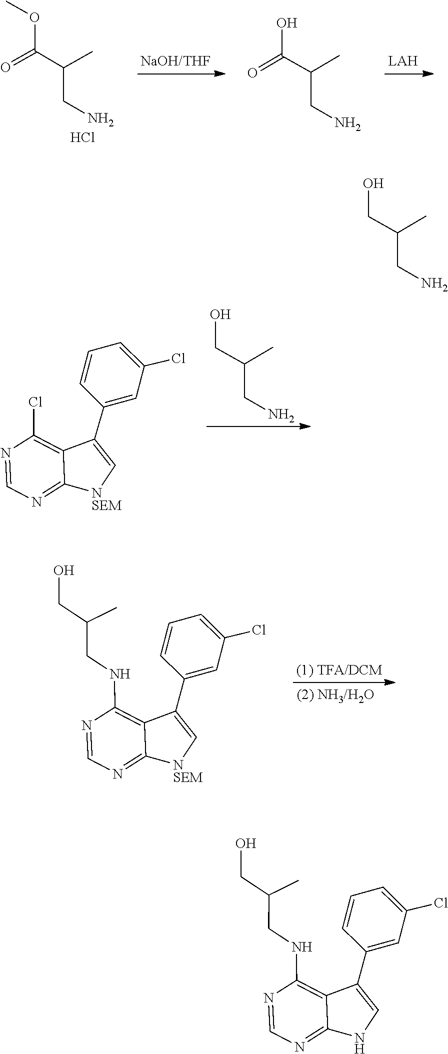

### 3-amino-2-methylpropanoic acid

To a solution of methyl 3-amino-2-methylpropanoate hydrochloride (1 g, 6.54 mmol, 1.0 equiv) in THF (10 mL) was added NaOH aqueous (784.3 mg, 19.61 mmol, 3.0 equiv) (784.3 mg in 2 mL H₂O). The resulting mixture was stirred at room temperature for 4 h. The mixture was quenched with H₂O (15 mL) and extracted with EA (10 mL×3). The combined layers organic were washed with brine (15 mL), dried with Na₂SO₄ and filtered. The filtrate was concentrated to give the crude product 3-amino-2-methylpropanoic acid (644 mg, 95.6%) as a white solid. LC-MS: 103.2 [M+H]⁺, purity >95% (214, 254 nm).

### 3-amino-2-methylpropan-1-ol

To a solution of methyl 3-amino-2-methylpropanoic acid (644 mg, 6.3 mmol, 1.0 equiv) in dry-THF (10 mL) was added LiAlH₄ (4.7 mL, 9.5 mmol, 1.5 equiv) (2 mol/L). The resulting mixture was stirred at 0° C. for 4 h. The reaction mixture was quenched with Na₂SO₄·10H₂O slowly. The resulting mixture was filtered by a pad of celite and the filtrate was concentrated to give the crude product 3-amino-2-methylpropan-1-ol (470 mg, 84.2%) as colorless oil. LC-MS: 89.1 [M+H]⁺, purity >85% (214, 254 nm).

To a solution of 3-((5-(3-chlorophenyl)-7-((2-(trimethylsilyl)ethoxy)methyl)-7H-pyrrolo[2,3-d]pyrimidin-4-yl)amino)-2-methylpropan-1-ol (134 mg, 0.30 mmol, 1.0 equiv) in DCM (5 mL) was added Trifluoroacetate (2.5 mL). The mixture was stirred at room temperature for 2 h. The reaction mixture was concentrated to give the crude pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 3-((5-(3-chlorophenyl)-7H-pyrrolo[2,3-d]pyrimidin-4-yl)amino)-2-methylpropan-1-ol (42.1 mg, 43.4%) as a white solid. LC-MS: 316.8 [M+H]⁺, purity >98% (214, 254 nm); ¹H NMR (400 MHZ, DMSO-d₆) δ 11.90 (s, 1H), 8.18 (s, 1H), 7.51-7.38 (m, 4H), 7.32 (s, 1H), 5.62 (t. J=5.6 Hz, 1H), 4.60-4.57 (m, 1H), 3.42 (t, J=6.0 Hz, 2H), 3.34-3.27 (m, 2H), 1.85-1.80 (m, 1H), 0.86 (d, J=6.8 Hz, 3H).

### Example 21: Synthesis of 3-((5-(3-chlorophenyl)-7H-pyrrolo[2,3-d]pyrimidin-4-yl)(methyl)amino)-2-methylpropan-1-ol (Compound 121)

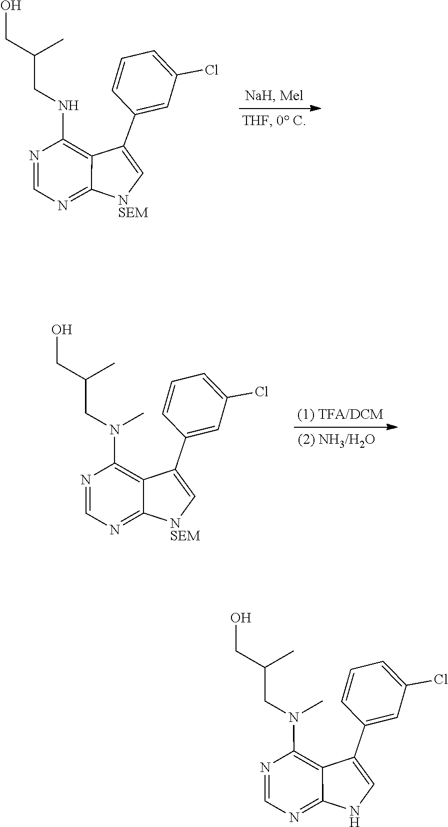 [SMILES: CC(CO)CNc1ncnc2c1c(-c1cccc(Cl)c1)cn2COCC[Si](C)(C)C ; CC(CO)CN(C)c1ncnc2c1c(-c1cccc(Cl)c1)cn2COCC[Si](C)(C)C ; CC(CO)CN(C)c1ncnc2[nH]cc(-c3cccc(Cl)c3)c12]

### 3-((5-(3-chlorophenyl)-7-((2-(trimethylsilyl)ethoxy)methyl)-7H-pyrrolo[2,3-d]pyrimidin-4-yl)(methyl)amino)-2-methylpropan-1-ol

To a solution of 3-((5-(3-chlorophenyl)-7-((2-(trimethylsilyl)ethoxy)methyl)-7H-pyrrolo[2,3-d]pyrimidin-4-yl)amino)-2-methylpropan-1-ol (200 mg, 0.45 mmol, 1.0 equiv) in dry-THF (5 mL) was added NaH (23.3 mg, 0.58 mmol, 1.3 equiv). The resulting mixture was stirred at for 0.5 h. A solution of Mel (129.2 mg, 0.67 mmol, 1.5 equiv) in THF was added drop wise at 0° C. The mixture was stirred at room temperature for 16 h. Upon completion, the mixture was quenched with H₂O (10 mL) and extracted with EA (5 mL×3). The combined organic layers were washed with brine (10 mL), dried with Na₂SO₄ and filtered. The filtrate was concentrated to give the crude product which was purified by flash column chromatography to afford 3-((5-(3-chlorophenyl)-7-((2-(trimethylsilyl)ethoxy)methyl)-7H-pyrrolo[2,3-d]pyrimidin-4-yl)(methyl)amino)-2-methylpropan-1-ol (68 mg, 33.0%) as a white solid. LC-MS: 461.1 [M+H]⁺, purity >95% (214, 254 nm). 3-((5-(3-chlorophenyl)-7H-pyrrolo[2,3-d]pyrimidin-4-yl)(methyl)amino)-2-methylpropan-1-ol

To a solution of 3-((5-(3-chlorophenyl)-7-((2-(trimethylsilyl)ethoxy)methyl)-7H-pyrrolo[2,3-d]pyrimidin-4-yl)(methyl)amino)-2-methylpropan-1-ol (67 mg, 0.15 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by reversed HPLC to afford the compound 3-((5-(3-chlorophenyl)-7H-pyrrolo[2,3-d]pyrimidin-4-yl)(methyl)amino)-2-methylpropan-1-ol (14.6 mg, 30.4%) as a white solid. LC-MS: 330.8 [M+H]⁺, purity >98% (214, 254 nm); ¹H NMR (400 MHZ, CDCl₃) δ 11.16 (s, 1H), 8.31 (s, 1H), 7.43 (s, 1H), 7.33-7.26 (m, 3H), 7.14 (s, 1H), 3.94-3.88 (m, 1H), 3.51 (dd. J=11.6, 3.2 Hz, 1H). 3.41-3.31 (m, 2H), 3.41 (s, 3H), 2.05-2.02 (m, 1H), 1.05 (d. J=6.8 Hz, 3H).

### Example 22: Synthesis of 5-(3-chlorophenyl)-N-(3-methoxy-2-methylpropyl)-N-methyl-7H-pyrrolo[2,3-d]pyrimidin-4-amine (Compound 122)

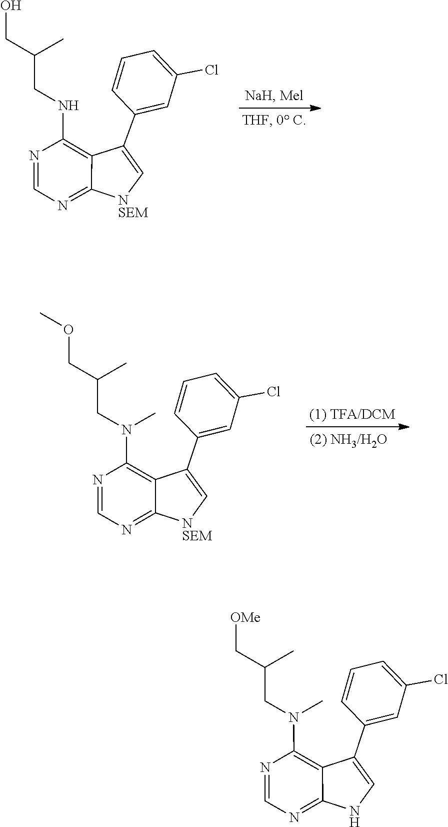 [SMILES: CC(CO)CNc1ncnc2c1c(-c1cccc(Cl)c1)cn2COCC[Si](C)(C)C ; COCC(C)CN(C)c1ncnc2c1c(-c1cccc(Cl)c1)cn2COCC[Si](C)(C)C ; COCC(C)CN(C)c1ncnc2[nH]cc(-c3cccc(Cl)c3)c12]

### 5-(3-chlorophenyl)-N-(3-methoxy-2-methylpropyl)-N-methyl-7-((2-(trimethylsilyl)ethoxy)methyl)-7H-pyrrolo[2,3-d]pyrimidin-4-amine

To a solution of 3-((5-(3-chlorophenyl)-7-((2-(trimethylsilyl)ethoxy)methyl)-7H-pyrrolo[2,3-d]pyrimidin-4-yl)amino)-2-methylpropan-1-ol (130 mg, 0.29 mmol, 1.0 equiv) in dry-THF (5 mL) was added NaH (26.8 mg, 0.67 mmol, 2.3 equiv). The resulting mixture was stirred at 0° C. for 0.5 h. A solution of Mel (168 mg, 0.87 mmol, 3.0 equiv) was added drop wise. The mixture was stirred at room temperature for 16 h. Upon completion, the mixture was quenched with H₂O (10 mL) and extracted with EA (5 mL×3). The combined organic layers were washed with brine (10 mL), dried with Na₂SO₄ and filtered. The filtrate was concentrated to give the crude product and purified by flash column chromatography to afford 5-(3-chlorophenyl)-N-(3-methoxy-2-methylpropyl)-N-methyl-7-((2-(trimethylsilyl)ethoxy)methyl)-7H-pyrrolo[2,3-d]pyrimidin-4-amine (85 mg, 61.6%) as a white solid. LC-MS: 475.1 [M+H]⁺, purity >95% (214, 254 nm).

### 5-(3-chlorophenyl)-N-(3-methoxy-2-methylpropyl)-N-methyl-7H-pyrrolo[2,3-d]pyrimidin-4-amine

To a solution of 5-(3-chlorophenyl)-N-(3-methoxy-2-methylpropyl)-N-methyl-7-((2-(trimethylsilyl)ethoxy)methyl)-7H-pyrrolo[2,3-d]pyrimidin-4-amine (83 mg, 0.17 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-N-(3-methoxy-2-methylpropyl)-N-methyl-7H-pyrrolo[2,3-d]pyrimidin-4-amine (11.2 mg, 18.7%) as a white solid. LC-MS: 344.8 [M+H]⁺, purity >98% (214, 254 nm); ¹H NMR (400 MHZ, CDCl₃) δ 10.11 (s, 1H), 8.36 (s, 1H), 7.46 (s, 1H), 7.39-7.26 (m, 3H), 7.00 (s, 1H), 3.44-3.40 (m, 2H), 3.24 (s, 3H), 3.20-3.23 (m, 2H), 2.76 (s, 3H), 2.22-2.14 (m, 1H), 0.88 (d. J=6.8 Hz, 3H).

### Example 23: Synthesis of 5-(3-chlorophenyl)-N-(3-methoxy-2-methylpropyl)-7H-pyrrolo[2,3-d]pyrimidin-4-amine (Compound 123)

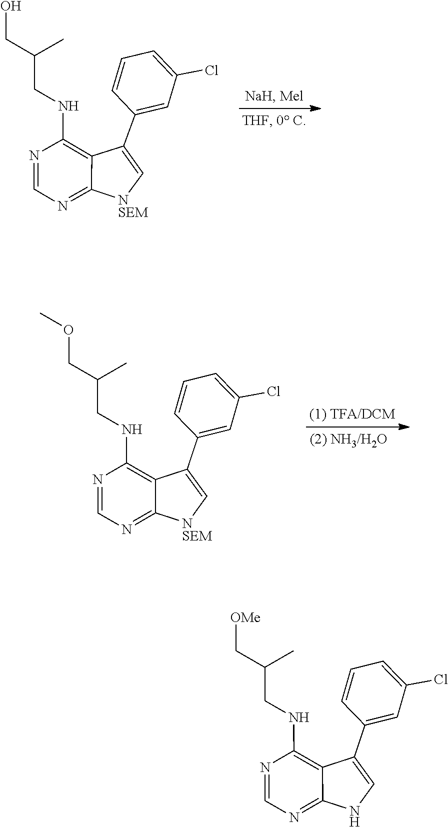 [SMILES: CC(CO)CNc1ncnc2c1c(-c1cccc(Cl)c1)cn2COCC[Si](C)(C)C ; COCC(C)CNc1ncnc2c1c(-c1cccc(Cl)c1)cn2COCC[Si](C)(C)C ; COCC(C)CNc1ncnc2[nH]cc(-c3cccc(Cl)c3)c12]

### 5-(3-chlorophenyl)-N-(3-methoxy-2-methylpropyl)-7-((2-(trimethylsilyl)ethoxy)methyl)-7H-pyrrolo[2,3-d]pyrimidin-4-amine

To a solution of 3-((5-(3-chlorophenyl)-7-((2-(trimethylsilyl)ethoxy)methyl)-7H-pyrrolo[2,3-d]pyrimidin-4-yl)amino)-2-methylpropan-1-ol (200 mg, 0.45 mmol, 1.0 equiv) in dry-THF (5 mL) was added NaH (23.3 mg, 0.58 mmol, 1.3 equiv). The resulting mixture was stirred at 0° C. for 0.5 h. A solution of Mel (129.2 mg, 0.67 mmol, 1.5 equiv) was added drop wise. The mixture was stirred at room temperature for 16 h. The mixture was quenched with H₂O (10 mL) and extracted with EA (5 mL×3). The combined organic layers were washed with brine (10 mL), dried with Na₂SO₄ and filtered. The filtrate was concentrated to give the crude product which was purified by flash column chromatography to afford 3-((5-(3-chlorophenyl)-7-((2-(trimethylsilyl)ethoxy) methyl)-7H-pyrrolo[2,3-d]pyrimidin-4-yl)(methyl)amino)-2-methylpropan-1-ol (62 mg, 30.1%) as a white solid. LC-MS: 461.1 [M+H]⁺, purity >95% (214, 254 nm).

### 5-(3-chlorophenyl)-N-(3-methoxy-2-methylpropyl)-7H-pyrrolo[2,3-d]pyrimidin-4-amine

To a solution of 5-(3-chlorophenyl)-N-(3-methoxy-2-methylpropyl)-7-((2-(trimethylsilyl)ethoxy)methyl)-7H-pyrrolo[2,3-d]pyrimidin-4-amine (62 mg, 0.13 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 5-(3-chlorophenyl)-N-(3-methoxy-2-methylpropyl)-7H-pyrrolo[2,3-d]pyrimidin-4-amine (7.3 mg, 16.6%) as a white solid. LC-MS: 330.8 [M+H]⁺, purity >98% (214, 254 nm); ¹H NMR (400 MHZ, CDCl₃) δ 11.71 (s, 1H), 8.40 (s, 1H), 7.51 (s, 1H), 7.39-7.32 (m, 3H), 7.01 (s, 1H), 5.62 (t, J=5.2 Hz, 1H), 3.70-3.64 (m, 1H), 3.50-3.43 (m, 1H), 3.31-3.22 (m, 1H), 3.22-3.05 (m, 1H), 3.33 (s, 3H), 2.09-2.04 (m, 1H), 0.96 (d, J=7.2 Hz, 3H).

### Example 24: Synthesis of 1-(5-(3-chlorophenyl)-7H-pyrrolo[2,3-d]pyrimidin-4-yl)piperidin-4-one (Compound 118)

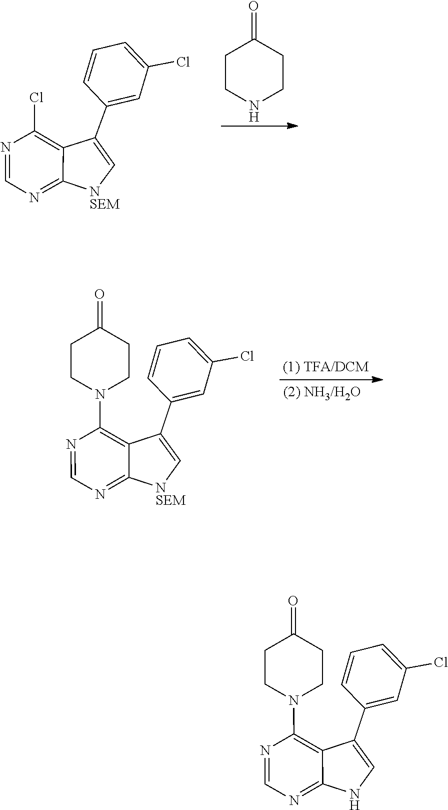

To a solution of 1-(5-(3-chlorophenyl)-7-((2-(trimethylsilyl)ethoxy)methyl)-7H-pyrrolo[2,3-d]pyrimidin-4-yl)piperidin-4-one (122 mg, 0.27 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 1-(5-(3-chlorophenyl)-7H-pyrrolo[2,3-d]pyrimidin-4-yl)piperidin-4-one (40.2 mg, 46.1%) as a white solid. LC-MS: 326.8 [M+H]⁺, purity >98% (214, 254 nm); ¹H NMR (400 MHZ, DMSO-d₆) δ 8.40 (s, 1H), 7.63 (d, J=3.6 Hz, 2H), 7.53-7.45 (m, 2H), 7.36-7.34 (m, 1H), 3.54 (t, J=6.0 Hz, 4H), 2.23 (t, J=6.0 Hz, 4H).

### Example 25: Synthesis of 3-((5-(3-chlorophenyl)-7H-pyrrolo[2,3-d]pyrimidin-4-yl)amino)propan-1-ol (Compound 119)

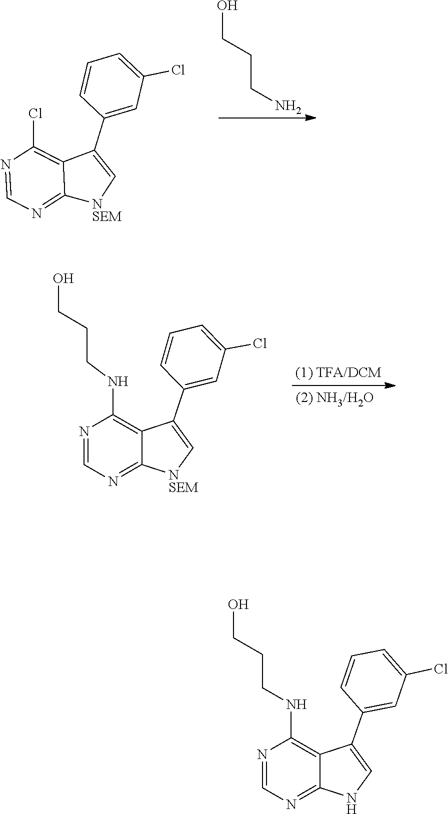 [SMILES: C[Si](C)(C)CCOCn1cc(-c2cccc(Cl)c2)c2c(Cl)ncnc21 ; NCCCO ; C[Si](C)(C)CCOCn1cc(-c2cccc(Cl)c2)c2c(NCCCO)ncnc21 ; OCCCNc1ncnc2[nH]cc(-c3cccc(Cl)c3)c12]

To a solution of 3-((5-(3-chlorophenyl)-7-((2-(trimethylsilyl)ethoxy)methyl)-7H-pyrrolo[2,3-d]pyrimidin-4-yl)amino)propan-1-ol (162 mg, 0.37 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 3-((5-(3-chlorophenyl)-7H-pyrrolo[2,3-d]pyrimidin-4-yl)amino)propan-1-ol (88.3 mg, 77.8%) as a white solid. LC-MS: 302.8 [M+H]⁺, purity >98% (214, 254 nm); ¹H NMR (400 MHZ, DMSO-d₆) δ 11.89 (s, 1H), 8.19 (s, 1H), 7.50-7.36 (m, 4H), 7.32 (s, 1H), 5.64 (t, J=4.8 Hz, 1H), 4.53 (t, J=4.8 Hz, 1H), 3.54-3.45 (m, 4H), 1.71-1.66 (m, 2H).

### Example 26: Synthesis of N-(1-(5-(3-chlorophenyl)-7H-pyrrolo[2,3-d]pyrimidin-4-yl)-4-methylpiperidin-4-yl)acetamide (Compound 124)

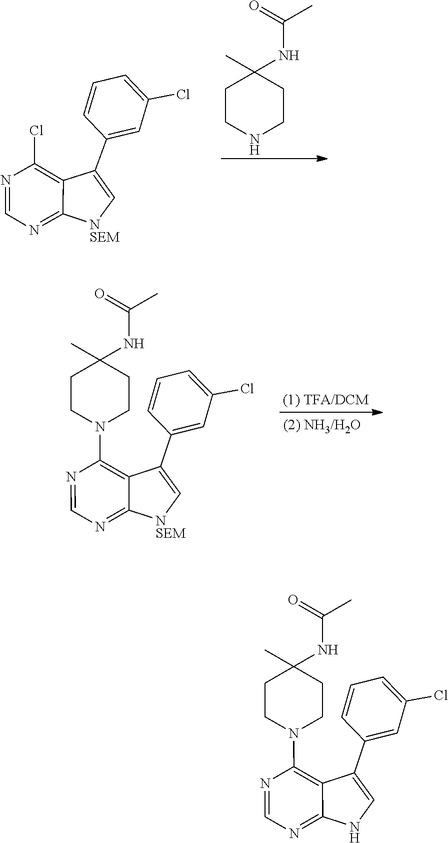

To a solution of N-(1-(5-(3-chlorophenyl)-7-((2-(trimethylsilyl)ethoxy)methyl)-7H-pyrrolo[2,3-d]pyrimidin-4-yl)-4-methylpiperidin-4-yl)acetamide (79 mg, 0.15 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by reversed HPLC to afford the compound N-(1-(5-(3-chlorophenyl)-7H-pyrrolo[2,3-d]pyrimidin-4-yl)-4-methylpiperidin-4-yl)acetamide (11.2 mg, 19.1%) as a white solid. LC-MS: 383.9 [M+H]⁺, purity >98% (214, 254 nm); ¹H NMR (400 MHZ, DMSO-d₆) δ 12.17 (s, 1H), 8.34 (d, J=2.8 Hz, 1H), 7.56 (d, J=2.8 Hz, 2H), 7.51-7.44 (m, 2H), 7.35-7.31 (m, 2H), 3.43 (d, J=13.6 Hz, 2H), 2.94 (t, J=12.0 Hz, 2H), 1.92 (d, J=13.6 Hz, 2H), 1.76 (s, 3H), 1.32-1.23 (m, 2H), 1.20 (s, 3H).

### Example 27: Synthesis of N¹,N³-bis(5-(3-chlorophenyl)-7H-pyrrolo[2,3-d]pyrimidin-4-yl)-2-methylpropane-1,3-diamine (Compound 132)

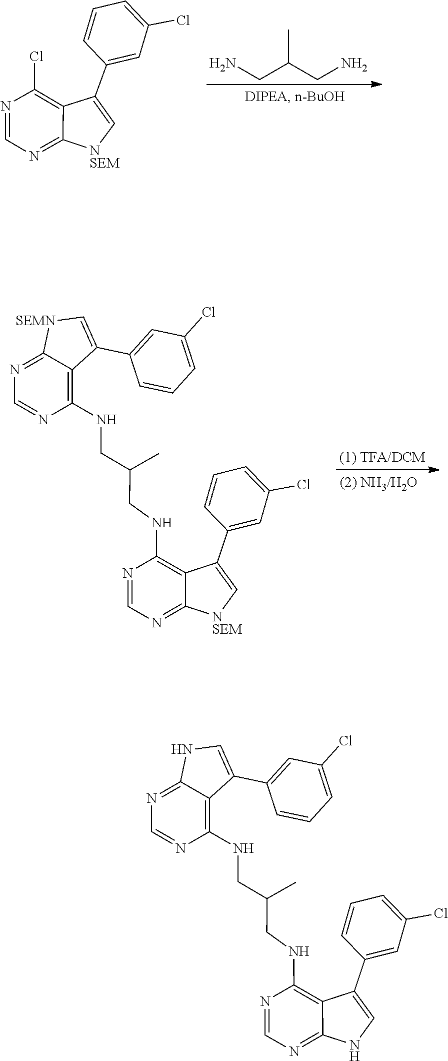

To a solution of N¹, N³-bis(5-(3-chlorophenyl)-7-((2-(trimethylsilyl)ethoxy)methyl)-7H-pyrrolo[2,3-d]pyrimidin-4-yl)-2-methylpropane-1,3-diamine (80 mg, 0.10 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound N-(1-(5-(3-chlorophenyl)-7H-pyrrolo[2,3-d]pyrimidin-4-yl)-4-methylpiperidin-4-yl)acetamide (26.7 mg, 49.4%) as a white solid. LC-MS: 543.4 [M+H]⁺, purity >98% (214, 254 nm); ¹H NMR (400 MHZ, DMSO-d₆) δ 11.88 (s, 2H), 7.79 (s, 2H), 7.55 (s, 2H), 7.54-7.34 (m, 4H), 7.34-7.32 (m, 4H), 5.97 (t, J=6.0 Hz, 2H), 3.55-3.49 (m, 2H), 3.47-3.40 (m, 2H), 2.07-1.99 (m, 1H), 0.98 (d, J=7.2 Hz, 3H).

### Example 28: Synthesis of 3-((5-(3-chlorophenyl)-7H-pyrrolo[2,3-d]pyrimidin-4-yl)amino)-2-methylpropanoic acid (Compound 126)

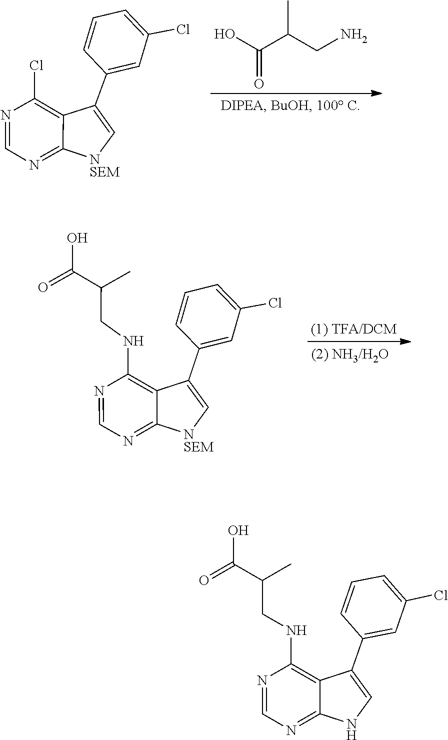

### 3-((5-(3-chlorophenyl)-7-((2-(trimethylsilyl)ethoxy)methyl)-7H-pyrrolo[2,3-d]pyrimidin-4-yl)amino)-2-methylpropanoic acid

The mixture of 4-chloro-5-(3-chlorophenyl)-7-((2-(trimethylsilyl)ethoxy)methyl)-7H-pyrrolo[2,3-d]pyrimidine (2.65 g, 0.10 mmol, 1.0 equiv), DIPEA (2.6 g, 6.74 mmol, 3.0 equiv) and 3-amino-2-methylpropanoic acid (1.04 g, 10.1 mmol, 1.5 equiv) in BuOH (30 mL) was stirred at 100° C. for 18 h. The mixture was concentrated to give the crude product which was purified by flash column chromatography to afford 3-((5-(3-chlorophenyl)-7-((2-(trimethylsilyl)ethoxy)methyl)-7H-pyrrolo[2,3-d]pyrimidin-4-yl)amino)-2-methylpropanoic acid (2.1 g, 67.7%) as a white solid. LC-MS: 543.4 [M+H]⁺, purity >95% (214, 254 nm).

### 3-((5-(3-chlorophenyl)-7H-pyrrolo[2,3-d]pyrimidin-4-yl)amino)-2-methylpropanoic acid

To a solution of 3-((5-(3-chlorophenyl)-7-((2-(trimethylsilyl)ethoxy)methyl)-7H-pyrrolo[2,3-d]pyrimidin-4-yl)amino)-2-methylpropanoic acid (50 mg, 0.11 mmol, 1.0 equiv) in DCM (5 mL) was added Trifluoroacetate (2.5 mL). The mixture was stirred at room temperature for 2 h and then the reaction mixture was concentrated to give pre-product which was diluted with a mixture of DCM (3 mL), methanol (3 mL) and 27% wt aqueous ammonia (3 mL). The resulting mixture was stirred at room temperature for 18 h. Upon completion, the mixture was concentrated to obtain the crude product which was purified by prep-HPLC to afford the compound 3-((5-(3-chlorophenyl)-7H-pyrrolo[2,3-d]pyrimidin-4-yl)amino)-2-methylpropanoic acid (20.4 mg, 56.9%) as a white solid. LC-MS: 330.8 [M+H]⁺, purity >98% (214, 254 nm); ¹H NMR (400 MHZ, DMSO-d₆) δ 11.92 (s, 1H), 8.20 (s, 1H), 7.52-7.45 (m, 2H), 7.39-7.33 (m, 3H), 5.68-5.71 (m, 1H), 3.63-3.55 (m, 2H), 2.73-2.66 (m, 2H), 1.10 (d. J=7.2 Hz, 3H).

### Example 29: Synthesis of 3-((5-(3-chlorophenyl)-7H-pyrrolo[2,3-d]pyrimidin-4-yl)amino)-2-methylpropanoic acid (Compound 124)

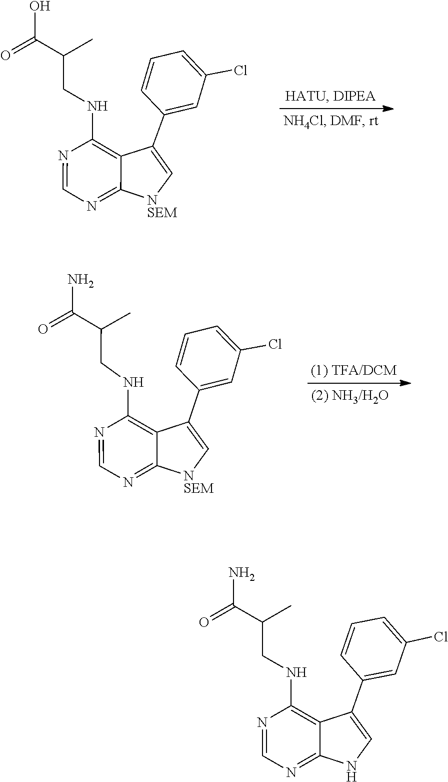 [SMILES: CC(CNc1ncnc2c1c(-c1cccc(Cl)c1)cn2COCC[Si](C)(C)C)C(=O)O ; CC(CNc1ncnc2c1c(-c1cccc(Cl)c1)cn2COCC[Si](C)(C)C)C(N)=O ; CC(CNc1ncnc2[nH]cc(-c3cccc(Cl)c3)c12)C(N)=O]

### 3-((5-(3-chlorophenyl)-7-((2-(trimethylsilyl)ethoxy)methyl)-7H-pyrrolo[2,3-d]pyrimidin-4-yl)amino)-2-methylpropanamide

The mixture of 4-chloro-5-(3-chlorophenyl)-7-((2-(trimethylsilyl)ethoxy)methyl)-7H-pyrrolo[2,3-d]pyrimidine (2.1 g, 4.56 mmol, 1.0 equiv), DIPEA (1.77 g, 13.7 mmol, 3.0 equiv), HATU (2.08 g, 5.48 mmol, 1.2 equiv) and Ammonium Chloride (732.7 mg, 13.7 mmol, 3.0 equiv) in DMF (10 mL) was stirred at room temperature for 4 h. The mixture was quenched with H₂O (50 mL) and extracted with EA (20 mL×3). The combined organic layers were washed with water (20 mL) and brine (20 mL), dried with Na₂SO₄. Filtered and the filtrate was concentrated to obtain the crude product which was purified by flash column chromatography to afford 3-((5-(3-chlorophenyl)-7-((2-(trimethylsilyl)ethoxy)methyl)-7H-pyrrolo[2,3-d]pyrimidin-4-yl)amino)-2-methylpropanamide (802 mg, 38.4%) as a white solid. LC-MS: 460.0 [M+H]⁺, purity >95% (214, 254 nm).

### 3-((5-(3-chlorophenyl)-7H-pyrrolo[2,3-d]pyrimidin-4-yl)amino)-2-methylpropanamide

To a solution of 3-((5-(3-chlorophenyl)-7-((2-(trimethylsilyl)ethoxy)methyl)-7H-pyrrolo[2,3-d]pyrimidin-4-yl)amino)-2-methylpropanamide (50 mg, 0.11 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound 3-((5-(3-chlorophenyl)-7H-pyrrolo[2,3-d]pyrimidin-4-yl)amino)-2-methylpropanamide (14.7 mg, 41.1%) as a white solid. LC-MS: 329.8 [M+H]⁺, purity >98% (214, 254 nm); ¹H NMR (400 MHZ. DMSO-d₆) δ 11.91 (s, 1H), 8.20 (s, 1H). 7.50-7.46 (m, 2H), 7.38-7.32 (m, 4H), 6.85 (s, 1H), 5.66 (t. J=5.6 Hz, 1H), 3.62-3.57 (m, 1H), 3.48-3.43 (m, 1H), 2.67-2.59 (m, 1H), 1.05 (d. J=7.2 Hz, 3H).

### Example 30: Synthesis of N¹-(5-(3-chlorophenyl)-7H-pyrrolo[2,3-d]pyrimidin-4-yl)-2-methylpropane-1,3-diamine (Compound 127)

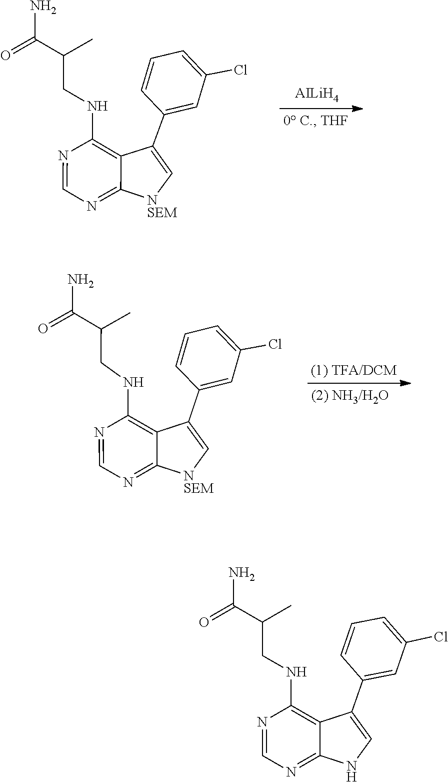 [SMILES: CC(CNc1ncnc2c1c(-c1cccc(Cl)c1)cn2COCC[Si](C)(C)C)C(N)=O ; CC(CNc1ncnc2c1c(-c1cccc(Cl)c1)cn2COCC[Si](C)(C)C)C(N)=O ; CC(CNc1ncnc2[nH]cc(-c3cccc(Cl)c3)c12)C(N)=O]

### N¹-(5-(3-chlorophenyl)-7-((2-(trimethylsilyl)ethoxy)methyl)-7H-pyrrolo[2,3-d]pyrimidin-4-yl)-2-methylpropane-1,3-diamine

To a solution of 3-((5-(3-chlorophenyl)-7-((2-(trimethylsilyl)ethoxy)methyl)-7H-pyrrolo[2,3-d]pyrimidin-4-yl)amino)-2-methylpropanamide (600 mg, 1.31 mmol, 1.0 equiv) in 5 mL dry-THF was added LiAlH₄ (1.0 mL, 1.96 mmol, 1.5 equiv) (2 mol/L) at 0° C. under N2. The resulting mixture was stirred at 0° C. for 5 h. The mixture was quenched with H₂O (10 mL) and extracted with EA (5 mL×3). The combined organic layers were washed with brine (10 mL), dried with Na₂SO₄ and filtered. The filtrate was concentrated to give the crude product which was purified by flash column chromatography to afford N¹-(5-(3-chlorophenyl)-7H-pyrrolo[2,3-d]pyrimidin-4-yl)-2-methylpropane-1,3-diamine (130 mg, 22.3%) as a light oil. LC-MS: 446.1 [M+H]⁺, purity >95% (214, 254 nm).

### N¹-(5-(3-chlorophenyl)-7H-pyrrolo[2,3-d]pyrimidin-4-yl)-2-methylpropane-1,3-diamine

To a solution of N¹-(5-(3-chlorophenyl)-7H-pyrrolo[2,3-d]pyrimidin-4-yl)-2-methylpropane-1,3-diamine (50 mg, 0.11 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound N¹-(5-(3-chlorophenyl)-7H-pyrrolo[2,3-d]pyrimidin-4-yl)-2-methylpropane-1,3-diamine (16.8 mg, 47.5%) as a white solid. LC-MS: 315.8 [M+H]⁺, purity >98% (214, 254 nm); ¹H NMR (400 MHZ, DMSO-d₆) δ 8.17 (s, 1H), 7.50-7.37 (m, 5H), 7.31 (d, J=4.8 Hz, 1H), 5.91 (t, J=10.4 Hz, 0.6H), 5.64 (t, J=11.2 Hz, 0.4H), 3.50-3.32 (m, 5H), 2.98-2.84 (m, 1H), 1.86-1.67 (m, 1H), 0.86 (d, J=3.2 Hz, 3H).

### Example 31: Synthesis of N-(3-((5-(3-chlorophenyl)-7H-pyrrolo[2,3-d]pyrimidin-4-yl)(methyl)amino)-2-methylpropyl)-N-methylacetamide (Compound 128)

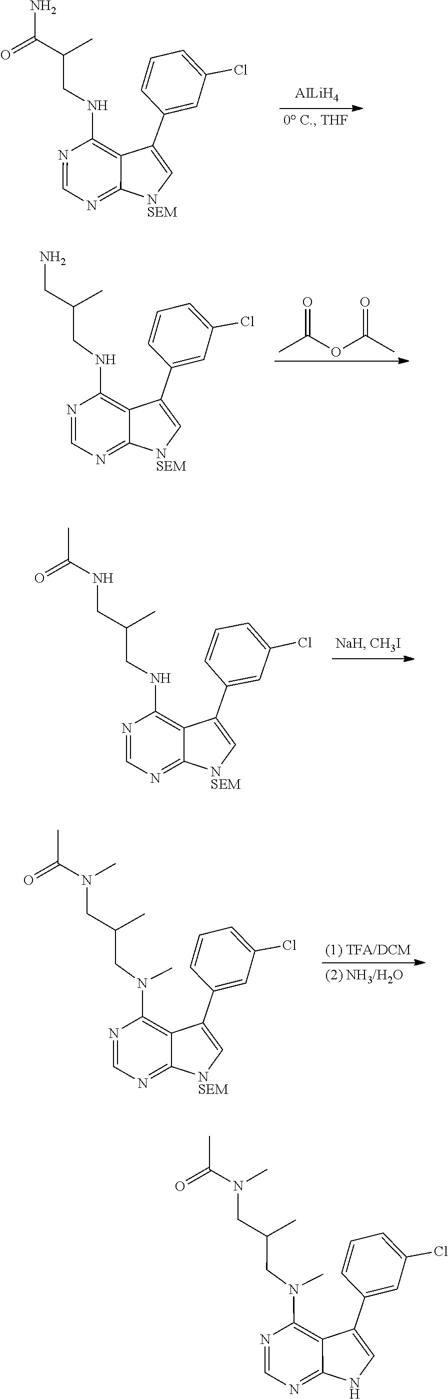

### N¹-(5-(3-chlorophenyl)-7-((2-(trimethylsilyl)ethoxy)methyl)-7H-pyrrolo[2,3-d]pyrimidin-4-yl)-2-methylpropane-1,3-diamine

To a solution of 3-((5-(3-chlorophenyl)-7-((2-(trimethylsilyl)ethoxy)methyl)-7H-pyrrolo[2,3-d]pyrimidin-4-yl)amino)-2-methylpropanamide (600 mg, 1.31 mmol, 1.0 equiv) in 5 mL dry-THF was added LiAlH₄ (1.0 mL, 1.96 mmol, 1.5 equiv) (2 mol/L). The resulting mixture was stirred at 0° C. for 5 h. The mixture was quenched with H₂O (10 mL) and extracted with EA (15 mL×3). The combined organic layers were washed with brine (10 mL), dried with Na₂SO₄ and filtered. The filtrate was concentrated to give the crude product which was purified by flash column chromatography to afford N¹-(5-(3-chlorophenyl)-7H-pyrrolo[2,3-d]pyrimidin-4-yl)-2-methylpropane-1,3-diamine (130 mg, 22.3%) as a light oil. LC-MS: 446.1 [M+H]⁺, purity >95% (214, 254 nm).

### N-(3-((5-(3-chlorophenyl)-7-((2-(trimethylsilyl)ethoxy)methyl)-7H-pyrrolo[2,3-d]pyrimidin-4-yl)(methyl)amino)-2-methylpropyl)-N-methylacetamide

To a solution of N-(3-((5-(3-chlorophenyl)-7-((2-(trimethylsilyl)ethoxy)methyl)-7H-pyrrolo[2,3-d]pyrimidin-4-yl)amino)-2-methylpropyl)acetamide (80 mg, 0.16 mmol, 1.0 equiv) in dry-THF (5 mL) was added NaH (15 mg, 0.38 mmol, 2.3 equiv) at 0° C. The resulting mixture was stirred at 0° C. for 0.5 h. A solution of Mel (94.6 mg, 0.49 mmol, 3.0 equiv) was added drop wise. The mixture was stirred at room temperature for 16 h. Upon completion, the mixture was quenched with H₂O (10 mL) and extracted with EA (5 mL×3). The combined organic layers were washed with brine (10 mL), dried with Na₂SO₄ and filtered. The filtrate was concentrated to give the crude product and purified by flash column chromatography to afford N-(3-((5-(3-chlorophenyl)-7-((2-(trimethylsilyl)ethoxy)methyl)-7H-pyrrolo[2,3-d]pyrimidin-4-yl)(methyl)amino)-2-methylpropyl)-N-methylacetamide (52 mg, 61.5%) as a white solid. LC-MS: 516.1 [M+H]⁺, purity >95% (214, 254 nm).

### N-(3-((5-(3-chlorophenyl)-7H-pyrrolo[2,3-d]pyrimidin-4-yl)(methyl)amino)-2-methylpropyl

To a solution of N-(3-((5-(3-chlorophenyl)-7-((2-(trimethylsilyl)ethoxy)methyl)-7H-pyrrolo[2,3-d]pyrimidin-4-yl)(methyl)amino)-2-methylpropyl)-N-methylacetamide (52 mg, 0.10 mmol, 1.0 equiv) in DCM (5 mL) was added Trifluoroacetate (2.5 mL). The mixture was stirred at room temperature for 2 h. The reaction mixture was concentrated to give the pre-product which was diluted with a mixture of DCM (3 mL), methanol (3 mL) and 27% wt aqueous ammonia (3 mL). The resulting mixture was stirred at room temperature for 18 h. The mixture was concentrated to obtain the crude product which was purified by prep-HPLC to afford the compound N-(3-((5-(3-chlorophenyl)-7H-pyrrolo[2,3-d]pyrimidin-4-yl)(methyl)amino)-2-methylpropyl)-N-methylacetamide (10.1 mg, 26.2%) as a white solid. LC-MS: 385.9 [M+H]⁺, purity >98% (214, 254 nm); ¹H NMR (400 MHZ. DMSO-d₆) δ 12.02 (s, 1H), 8.25 (d. J=6.4 Hz, 1H), 7.51-7.32 (m, 6H), 3.29-3.06 (m. 4H), 2.89 (s, 1.8H), 2.72 (s, 1.2H), 2.65 (d. J=7.2 Hz, 3H), 2.28-2.15 (m, 1H), 1.95 (s, 1.7H), 1.88 (s, 1.3H), 0.80-0.74 (m, 3H).

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

### BIOLOGICAL EXAMPLES

### Example B-1: Protein Expression and Purification

STK3 and STK4 kinase domains were expressed as recombinant fusion proteins incorporating a His6 tag at the N-terminus. E, coli were cultured in Terrific Broth (TB) media at 37° C. until an OD600 of ˜1.8 was reached. The culture was then cooled to 18° C. and allowed to reach at OD600 of ˜2.6. Protein production was induced by the addition of 0.5 mM Isopropyl β-D-1-thiogalactopyranoside (IPTG) overnight. Cells were harvested and lysed by sonication, and recombinant proteins were purified using Ni2+- or Co2+-affinity chromatography. The eluted proteins were buffer exchanged into 50 mM Tris pH 7.5, 500 mM NaCl, 1.0 mM TCEP, and 5% glycerol, and their expression tags were cleaved using Tobacco Etch Virus (TEV) protease. Where necessary, autophosphorylation was performed during proteolysis by the addition of ATP and MgCl₂ at 10- and 20-fold molar excess, respectively. The cleaved proteins were passed through Ni2+ beads and further purified by size exclusion chromatography. The pure proteins in 20 mM Tris pH 7.5, 150 mM NaCl, and 0.5 mM tris(2-carboxyethyl) phosphine (TCEP) were stored at −80° C.

### Example B-2: ADP-Glo Kinase Assay

The STK3 kinase assay was performed in 5 μL reaction buffer containing 50 ng recombinant human STK3 protein (full length, SignalChem #S24-10G; Richmond, Canada), 250 μg/mL myelin basic protein (Sigma-Aldrich #M1891; St. Louis, MO, USA), and 50 μM ATP (Sigma-Aldrich #A7699). The STK4 kinase assay was performed in 5 μL reaction buffer containing 50 ng recombinant human STK4 protein (full length, SignalChem #S25-10G), 300 μg/mL Axltide (SignalChem #A16-58), and 50 μM ATP. IC₅₀ values were determined with 10 concentrations of compounds serially diluted 3-fold from a starting concentration of 30 μM. Staurosporine, a non-selective protein kinase inhibitor, was included in the assay as a positive control. Three experiments were performed, each in triplicate.

Representative data for exemplary compounds disclosed herein are presented in Table 2.

TABLE 2
Inhibition of STK3 and STK4 in ADP-Glo kinase assay.

| Cmpd No. | STK4 IC₅₀ (μM)^{a} | STK3 IC₅₀ (μM)^{a} |
|---|---|---|
| 1 | A | A |
| 2 | B | C |
| 3 | A | A |
| 4 | A | A |
| 5 | A | A |
| 6 | A | A |
| 7 | A | A |
| 8 | A | A |
| 9 | A | B |
| 10 | A | A |
| 11 | A | A |
| 12 | A | A |
| 13 | A | A |
| 14 | A | A |
| 15 | A | A |
| 16 | A | A |
| 17 | A | A |
| 18 | A | A |
| 19 | B | B |
| 20 | C | C |
| 21 | C | C |
| 22 | C | C |
| 23 | A | A |
| 24 | B | B |
| 25 | B | A |
| 26 | A | B |
| 27 | B | B |
| 28 | B | B |
| 29 | B | C |
| 30 | B | B |
| 31 | B | B |
| 32 | B | C |
| 33 | C | C |
| 34 | C | C |
| 35 | C | C |
| 36 | C | C |
| 37 | C | C |
| 38 | C | C |
| 39 | C | C |
| 40 | C | C |
| 41 | A | B |
| 42 | A | B |
| 43 | C | C |
| 44 | A | A |
| 45 | A | B |
| 46 | B | B |
| 47 | B | B |
| 48 | C | C |
| 49 | A | A |
| 50 | A | A |
| 51 | A | A |
| 52 | A | A |
| 53 | A | A |
| 54 | A | A |
| 55 | A | B |
| 56 | C | C |
| 57 | C | C |
| 58 | C | C |
| 59 | A | B |
| 60 | A | B |
| 61 | A | B |
| 62 | A | A |
| 63 | A | B |
| 64 | B | B |
| 65 | B | B |
| 66 | B | B |
| 67 | B | C |
| 68 | B | B |
| 69 | B | B |
| 70 | B | C |
| 71 | B | C |
| 72 | B | C |
| 73 | B | C |
| 74 | C | C |
| 75 | C | C |
| 76 | C | C |
| 77 | C | C |
| 78 | C | C |
| 79 | C | C |
| 80 | C | C |
| 81 | B | C |
| 82 | C | C |
| 83 | C | C |
| 84 | C | C |
| 85 | C | C |
| 86 | C | C |
| 87 | C | C |
| 88 | C | C |
| 89 | C | C |
| 90 | C | C |
| 91 | C | C |
| 92 | C | C |
| 93 | C | C |
| 94 | C | C |
| 95 | C | C |
| 96 | A | B |
| 97 | C | C |
| 98 | A | A |
| 99 | C | C |
| 100 | A | B |
| 101 | C | C |
| 102 | A | A |
| 103 | A | A |
| 104 | A | A |
| 105 | A | A |
| 106 | A | A |
| 107 | A | A |
| 108 | A | A |
| 109 | A | A |
| 110 | A | A |
| 114 | A | A |
| 115 | A | A |
| 116 | A | A |
| 117 | A | A |
| 118 | A | B |
| 119 | A | A |
| 120 | C | C |
| 121 | A | A |
| 122 | B | B |
| 123 | A | A |
| 124 | B | B |
| 125 | A | A |
| 126 | B | B |
| 127 | A | A |
| 128 | B | A |
| 129 | A | A |
| 130 | B | B |
| 131 | C | C |

^{a}IC₅₀ values were determined using an ADP-Glo kinase assay. Mean of three technical replicates A: IC₅₀ is ≤1 μM; B: IC₅₀ is >1 μM and <5 μM; C: IC₅₀ ≥5 μM, and NT is not tested.
^{b}IC₅₀ values were determined using an NanoBRET TE intracellular kinase assay ± SEM. Mean of two technical replicates A: IC₅₀ is ≤1000 nM; B: IC₅₀ is >2000 nM and <1000 nM; C: IC₅₀ ≥2000 nM

### Example B-3: PhosphoSens CSox-Based Sensor Assay

Materials were obtained and prepared according to the assay manufacturer's protocol (Assay Quant Technologies: Marlborough, MA, USA). The 50 μL reaction was initiated by addition of a master mix containing the compounds or vehicle, kinase, and CSox-peptide substrate to a well containing the remaining components. Compounds were pre-incubated with the kinase for 15 min prior to adding CSox-peptide substrate. Final concentrations were: 50 FmM HEPES pH 7.5, 50 μM ATP, 1.0 mM DTT, 0.01% Brij-35, 0.5 mM EGTA, 10 mM MgCl2, 10 μM peptide substrate (AQT0135), and 5.0 nM STK3 (SignalChem #S24-10G) or STK4 (SignalChem #S25-10G) kinases. Samples and standards were plated in triplicate in half-area 96-well, white, flat- or round-bottomed NBS microplates (Corning #3994: Corning, NY, USA). IC₅₀ values were determined with 10 concentrations of compounds serially diluted 3-fold from a starting concentration of 30 μM. Fluorescence measurements (relative fluorescent units, RFU) were recorded every 2 min for up to 150 min at 30° C. using a Spark® multimode microplate reader (Tecan: Mannedorf, Switzerland) at 360/485 nm excitation/emission wavelengths. Background-corrected RFU were plotted against time using Prism 8 software. Velocity determinations were calculated for each reaction and plotted versus compound concentration to generate inhibitor curves.

To validate these initial findings, a second in vitro kinase assay was used with an orthogonal readout. The PhosphoSens® CSox-based Sensor real-time kinetic assay uses a peptide substrate containing an unnatural amino acid and physiological Mg²⁺ levels to quantify substrate phosphorylation using fluorescence. Experiments to determine optimal assay conditions indicated an optimal STK3/4 protein concentration of 5.0 nM and an optimal ATP Kₘ of 75 μM for STK3 and 140 μM for STK4. Compounds 6, 11, or 23 were titrated against STK3 or STK4 in the presence of Kₘ ATP for 2 h. Plots of the initial slopes against the log of the compound concentration were generated, IC₅₀ values were determined, and the Kᵢ values were calculated (Table 3). We found that compounds 6, 11, and 23 potently inhibited both kinases and showed better selectivity for STK4 over STK3.

TABLE 3
Inhibition of STK3 protein thermal shift and kinase activity.

| | Compound | | |
|---|---|---|---|
| | 6 | 11 | 23 |
| STK3: Kᵢ [nM] | 88 | 145 | 209 |
| STK4: Kᵢ [nM] | 52 | 91 | 33 |

Mean (n = 2).
Kᵢ was determined using the PhosphoSens CSox-based Sensor Assay.

### Example B-4: Thermal Shift Assay

Recombinant kinase domains of STK3 and STK4 at 2.0 μM in 10 mM HEPES pH 7.5 and 500 mM NaCl were mixed with 10 UM of the test inhibitors. Temperature-dependent protein unfolding profiles were measured using a Real-Time PCR Mx3005p machine (Stratagene: San Diego, CA, USA). Data evaluation and melting temperature calculation were performed using Protein Thermal Shift™ Software v1.2.

To further evaluate binding of these compounds to STK3/4, differential scanning fluorimetry (protein thermal shift using ThermoFluor technology) assay was used to benchmark the capacity to stabilize the kinase domain. The melting temperature (Tₘ) of STK3 or STK4 was measured in the presence of vehicle (DMSO) or 6, 11 or 23. A Tₘ shift of >7.4° C. was obtained for all three potent compounds tested, and comparable results were obtained for compounds 5, 7, 15, and 16. The thermal shift results corresponded closely to the potencies measured using the ADP-Glo kinase assay (Table 4).

TABLE 4
Inhibition of STK3 protein thermal shift and kinase activity.

| | Compound | | | | | |
|---|---|---|---|---|---|---|
| | 6 | 7 | 11 | 15 | 16 | 23 |
| ΔTₘ (° C) | 8.1 ± | 8.4 ± | 7.4 ± | 8.3 ± | 8.0 ± | 7.9 ± |
| | 0.1 | 0.02 | 0.2 | 0.1 | 0.3 | 0.1 |

Mean ± SEM (n = 3).
ΔTₘ was determined using a ThermoFluor differential scanning fluorimetry assay.

### Example B-5: Isothermal Titration Calorimetry (ITC)

All ITC experiments were performed using NanoITC (TA Instruments: New Castle, DE, USA) at 25° C. in a buffer containing 20 mM Tris pH 7.5, 150 mM NaCl, and 0.5 mM TCEP. Proteins at 12 μM were titrated into the reaction cell containing the inhibitors. For this protocol the chamber was pre-equilibrated with the test compound, and the kinase domain of STK3 or STK4 was titrated in while continuously measuring the rate of exothermic heat evolution. The heat of binding was integrated, corrected, and fitted to an independent single-binding site model based on the manufacturer's protocol, from which thermodynamic parameters (ΔH and TΔS), equilibrium association and dissociation constants (Kₐ and K_{D}), and stoichiometry (n) were calculated.

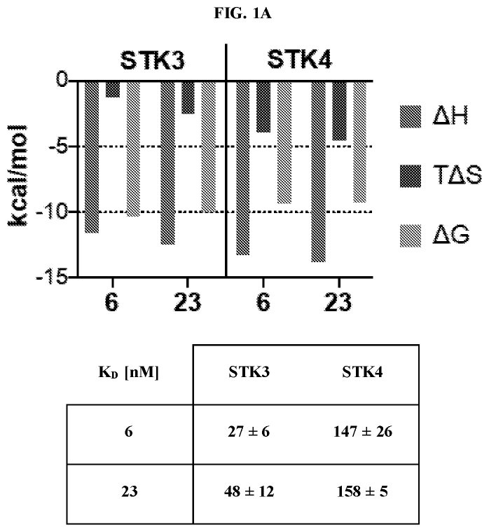

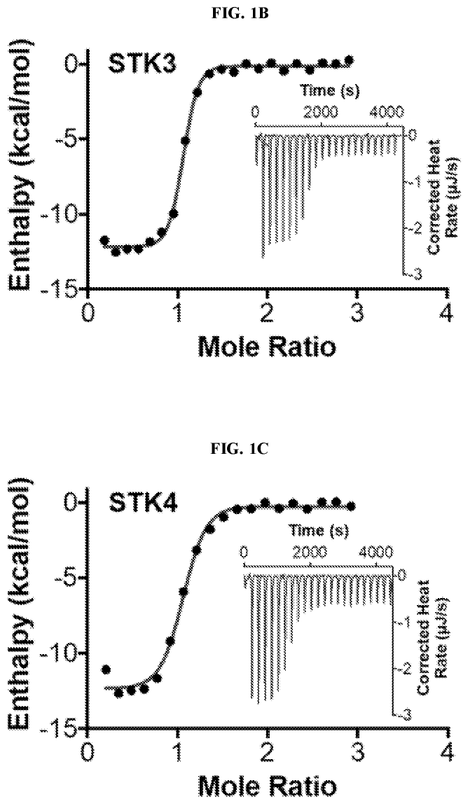

Isothermal titration calorimetry (ITC) was used to determine the thermodynamic properties of the compounds. Analysis of compounds 6 and 23 with STK3 and STK4 demonstrated low nanomolar affinities (ITC-K_{D}) that were comparable to the kinetic measurements (FIG. 1A). Both compounds exhibited enthalpy-driven thermodynamic profiles in their binding to STK3 and 4. Apart from strongly favorable binding enthalpy, entropy values are also favorable (FIG. 1B and FIG. 1C).

### Example B-6: NanoBRET Intracellular Kinase Assay

Full-length STK3, STK4, and LRRK2 kinase was cloned in-frame with a C-terminal NanoLuc-fusion (Promega: Madison, WI, USA) and the plasmid was transfected into HEK293T cells using FuGENE HD (Promega, E2312). After 20 h, transfected cells were mixed with inhibitors and 50 nM NanoBRET Kinase Tracer K10 (Promega) and reseeded at a density of 2× 105 cells/mL in Opti-Minimal Essential Medium (MEM) without phenol red (Life Technologies; Carlsbad, CA, USA) in 384-well plates (Greiner 781-207; Kremsmunster, Austria). After 2 h at 37° C. in a 5% CO₂ atmosphere, NanoBRET NanoGlo Substrate+Extra-cellular NanoLuc Inhibitor (Promega, N2540) was added, and filtered luminescence was measured on a PHERAstar plate reader (BMG Labtech; Ortenberg, Germany) equipped with a lumi-nescence filter pair (450 nm BP filter (donor) and 610 nm LP filter (acceptor)). Competitive displacement data were graphed using a normalized 3-parameter curve fit with the equation Y=100/(1+10\hat{ }[X−Log IC₅₀]) in Prism 8 software.

To confirm cellular potency for compounds described herein, the ADP-Glo kinase assay were used to detect engagement of STK3 and STK4 in live cells using the NanoBRET™ intracellular kinase assay, marking the first report of a NanoBRET assay for STK3 and STK4. This assay measured the apparent affinity of test compounds by competitive displacement of the NanoBRET™ tracer, reversibly bound to a NanoLuc® luciferase-kinase fusion in cells. To evaluate potential off-target activity of the inhibitors, the inhibition of LRRK2 in live cells by the STK3/4 inhibitors compounds described herein were measured. Cultured HEK293 cells were either transfected with NanoLuc®-STK3 fusion vector, NanoLuc®-STK4 fusion, or with NanoLuc®-LRRK2 vector for 24 h. A 50 nM tracer and these compounds were added to cells and incubated for 2 h. These compounds tested in the assays decreased the NanoBRET™ signal for both STK3 and STK4 at varying potencies and confirming cellular target engagement of STK3/4. The data indicated that the compounds could be a useful chemical probe.

TABLE 5
Cellular target engagement of compounds
against STK3, STK4 and LRRK2.

| | STK3: IC₅₀ | STK4: IC₅₀ | LRRK2: IC₅₀ |
|---|---|---|---|
| Compound | (nM)^{a} | (nM)^{a} | (nM)^{b} |
| 5 | A | A | A |
| 6 | A | A | A |
| 7 | A | A | A |
| 11 | A | A | C |
| 15 | B | A | B |
| 16 | A | A | B |
| 106 | A | A | C |
| 105 | A | A | B |
| 108 | A | A | B |
| 119 | A | A | C |
| 121 | A | A | C |
| 23 | A | A | A |

^{a}IC₅₀ values were determined using a NanoBRET TE kinase assay. Mean of three technical replicates A: IC₅₀ is ≤1 μM; B: IC₅₀ is >1 μM and <5 μM; C: IC₅₀ ≥5 μM, and NT is not tested.
^{b}IC₅₀ values were determined using a NanoBRET TE intracellular kinase assay ± SEM. Mean of two technical replicates A: IC₅₀ is ≤1000 nM; B: IC₅₀ is >2000 nM and <1000 nM; C: IC₅₀ ≥2000 nM

### Example B-7: Western Blot Analysis of p-MOB1 in HEK293 Cells

HEK293 cells were resuspended in DMEM Glutamax supplemented with 10% FBS, antibiotic/antimycotic, and sodium pyruvate (Gibco, Thermo Fisher Scientific) at 1.5×10⁵ cells/500 μL/well in 24-well tissue culture plates for 24 h. The medium was then replaced with fresh medium containing DMSO or 10 μM compound, and the cells were incubated for an additional 4 h. The medium was aspirated and replaced with medium containing 10 μM compound and 50 μM H₂O₂, and the cells were incubated for 2 h. Cells were harvested by pipetting, pelleted by centrifugation at 1400 rpm for 4 min, and lysed in RIPA buffer for 10 min at room temperature. The lysates were centrifuged at 12,000×g at room temperature (RT) for 10 min and the supernatants were recovered.

Protein concentrations in the supernatants were determined using a BCA assay (Pierce/Thermo Scientific). Proteins were separated on Novex™ WedgeWell™ 4-20% Tris-Glycine Gels (Invitrogen; Carlsbad, CA, USA) and transferred to polyvinylidene difluoride (Trans-Blot® Turbo™ Transfer Pack) membranes. Membranes were blocked in Tris-buffered saline Tween® 20 (50 mM Tris-HCl pH 7.6, 150 mM NaCl, 0.05% Tween 20; TBST) containing 3% Bovine Serum Albumin (BSA) for 1 h at room temperature and incubated overnight at 4° C. with primary antibodies as follows: 1:1000 dilution of anti-MOB1 (rabbit monoclonal antibody [mAb]#13730), 1:1000 anti-phospho-MOB1 Thr35 (rabbit mAb #8699), or 1:5000 anti-GAPDH (rabbit mAb #2118; all from Cell Signaling Technology; Danvers. MA. USA). Blots were rinsed three times with TBST and then incubated in 5% nonfat milk containing horseradish peroxidase (HRP)-conjugated mouse anti-rabbit IgG secondary Ab (Cell Signaling Technology) for 1 h at RT. Blots were washed three times with TBST, developed using Pierce® ECL Western Blotting reagent, and imaged and analyzed using with ImageJ software. Quantitative data are expressed as the level of phosphorylated MOB1 normalized to total MOB1.

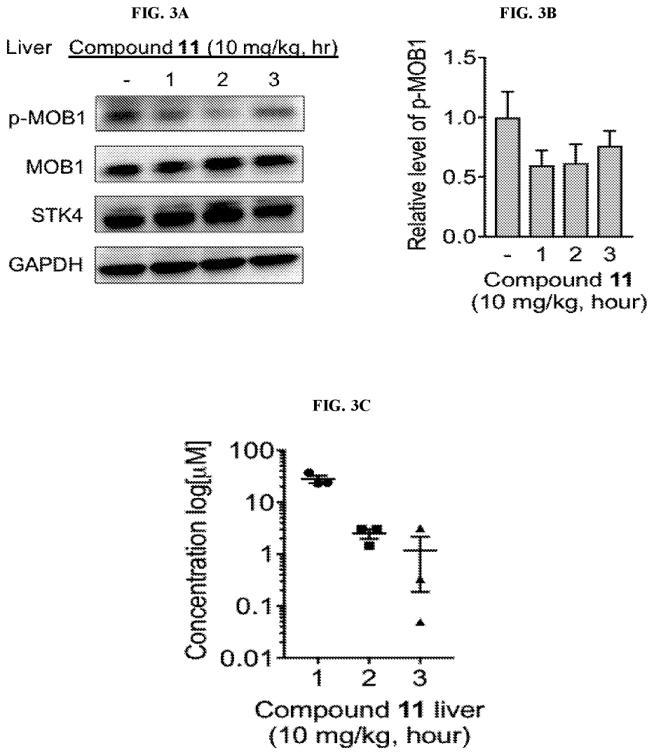

To confirm that the cellular mechanism of action of our inhibitors was through inhibition of Hippo pathway signaling. HEK293 cells were treated with DMSO or compounds 2, 6, 7, 9, 11, 15, 16, 106, 105, 108, 119, 121, and 23 (10 μM, 4 h) followed by treatment with H₂O₂ (50 μM, 2 h) to activate STK3/4. Inhibition of STK3/4 in vitro and in vivo can produce decreased phosphorylation of the Hippo pathway adapter protein MOB1 at Thr35. A Western blot analysis of HEK293 cell lysates demonstrated a significant reduction in the relative expression level of phosphorylated MOB1 by potent compounds, 2, 6, 7, 9, 11, 15, 16, 106, 105, 108, 119, 121, and 23 and compared with the DMSO control (FIG. 3A), however, weaker compounds showed no significant effect on the relative levels of p-MOB1.

### Example B-8: YAP-TAZ-TEAD Luciferase Assay

8×GTIIC-luciferase was a gift from Stefano Piccolo (Addgene plasmid #34615, http://n2t.net/addgene: 34615: RRID: Addgene_34615). HEK293 cells were transfected with 8×GTIIC-luciferase plasmid and control Renilla luciferase plasmid using jetPRIME transfection reagent (Polyplus-transfection RSA: New York, NY. USA) for 24 h and cultured in Dulbeco's Modified Eagle Media (DMEM) Glutamax supplemented with 10% Fetal Bovine Serum (FBS), antibiotic/antimycotic, and sodium pyruvate (Gibco. Thermo FisherScientific: Waltham. MA. USA). The transfected cells were treated with 10 μM compound in triplicate and incubated for 48 h at 37° C. Firefly and Renilla luminescence were detected with a Dual-Glo Luciferase Assay System (Promega #E2920) according to the manufacturer's recommendations. The ratio of firefly to Renilla luminescence was calculated for each well and normalized to the ratio in control cells incubated with DMSO.

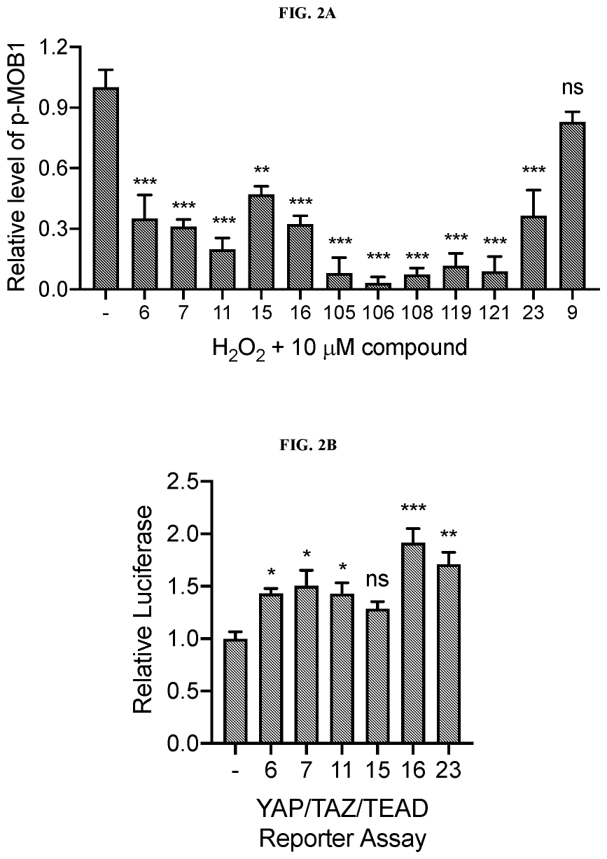

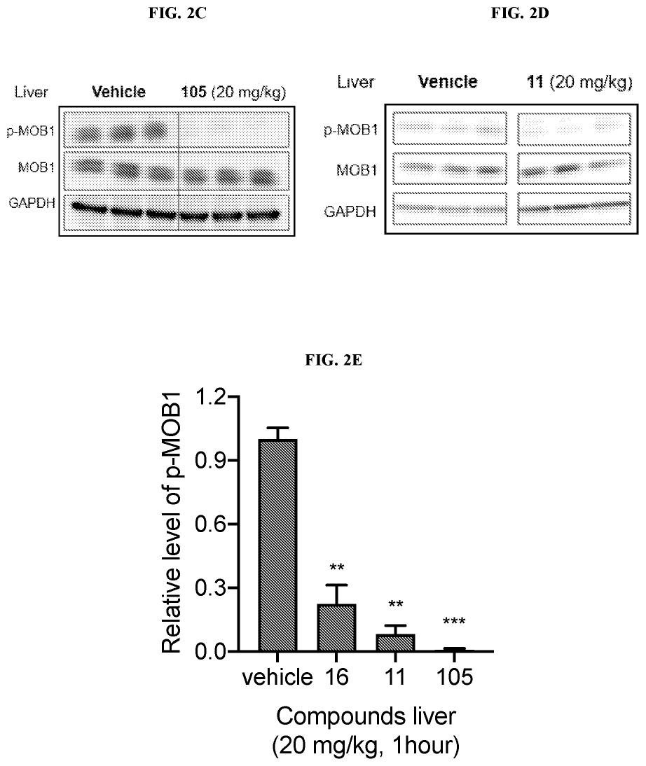

STK3/4 can negatively regulate the activity of the YAP/TAZ-TEAD protein complex and, accordingly, inactivation of these kinases reduces the transcription of YAP/TAZ-TEAD target genes. Therefore, the effects of compounds 2, 6, 7, 9, 11, 15, 16, 23 were examined in a luciferase reporter assay driven by 8×GTIIC, a YAP-TAZ-TEAD-responsive synthetic promoter. HEK293 cells were transfected with the 8×GTIIC-firefly luciferase plasmid and a control Renilla luciferase plasmid for 24 h, and then treated with DMSO or compound (10 μM) for 48 h before quantitation of the relative luciferase signal. Notably, our potent compounds, 6, 7, 11, 16, and 23, resulted in a marked increase in YAP/TAZ-TEAD activity compared with the DMSO control (FIG. 2B). The results confirmed the intracellular on-target activity of our potent compounds for inhibiting STK3 and STK4 activity and targeting gene expression in the Hippo signaling pathway.

### Example B-9: In Vivo Studies

All animal procedures were approved by the Sanford Burnham Prebys Medical Discovery Institute Institutional Animal Care and Use Committee and were performed according to the NIH guidelines for the Care and Use of Laboratory Animals. Adult female C₅₇BL/6J mice were purchased from Jackson Laboratories and housed with free access to food and water on a 12 h light/dark cycle. Compounds were formulated in 5% DMSO, 10% Tween-80, and 85% H2O and injected intraperitoneally at 10 or 20 mg/kg. Blood samples were collected retro-orbitally at the indicated times and plasma was prepared by centrifugation. Livers were collected post mortem at the indicated times.

For quantification of compound, plasma samples were extracted with acetonitrile: water 4:1 with 0.1% formic acid containing indomethacin as an internal standard. Samples were centrifuged and the supernatants were diluted with acetonitrile: water and analyzed by LC-MS/MS using a Shimadzu Nexera X2 High-performance liquid chromatography (HPLC) (Shimadzu Scientific Instruments: Kyoto, Japan) coupled to an AB Sciex 6500 QTRAP (AB Sciex LLC: Toronto, Canada).

### Example B-10: Western Blot Analysis of p-MOB1 in Mouse Liver

Mice were intraperitoneally injected with 20 mg/kg vehicle or compound and the livers were harvested at 1, 2, or 3 h after injection. Approximately 50 mg of liver tissue was placed in 1 mL Radioimmunoprecipitation assay (RIPA) buffer (50 mM Tris-HCl pH 8.0, 150 mM NaCl, 1.0% IGEPAL® CA-630, 0.5% sodium deoxycholate, and 0.1% SDS) containing phosphatase and protease inhibitor cocktails, homogenized with a PowerGen 125 homogenizer (Fisher Scientific), and then sonicated for 10 s. Homogenized tissues were centrifuged at 14,000×g at 4° C. for 20 min and the supernatants were recovered.

Protein concentrations in the supernatants were determined using a BCA assay (Pierce/Thermo Scientific). Proteins were separated on Novex™ WedgeWell™ 4-20% Tris-Glycine Gels (Invitrogen: Carlsbad, CA, USA) and transferred to polyvinylidene difluoride (Trans-Blot® Turbo™ Transfer Pack) membranes. Membranes were blocked in Tris-buffered saline Tween® 20 (50 mM Tris-HCl pH 7.6, 150 mM NaCl, 0.05% Tween 20: TBST) containing 3% Bovine Serum Albumin (BSA) for 1 h at room temperature and incubated overnight at 4° C. with primary antibodies as follows: 1:1000 dilution of anti-MOB1 (rabbit monoclonal antibody [mAb]#13730), 1:1000 anti-phospho-MOB1 Thr35 (rabbit mAb #8699), or 1:5000 anti-GAPDH (rabbit mAb #2118: all from Cell Signaling Technology: Danvers, MA, USA). Blots were rinsed three times with TBST and then incubated in 5% nonfat milk containing horseradish peroxidase (HRP)-conjugated mouse anti-rabbit IgG secondary Ab (Cell Signaling Technology) for 1 h at RT. Blots were washed three times with TBST, developed using Pierce® ECL Western Blotting reagent, and imaged and analyzed using with ImageJ software. Quantitative data are expressed as the level of phosphorylated MOB1 normalized to total MOB1.

The pharmacokinetic (PK) and pharmacodynamic (PD) properties of compounds 5, 6, 7, 11, 15, 16, 102, 106, 105, 108, 119, 121, and 23 was evaluated in vivo. Adult female C₅₇BL/6 mice (n=3/group) were intraperitoneally injected with vehicle or test compound (20 mg/kg), and blood samples and livers were collected at 30 min and 1 h post-injection, respectively. Plasma concentrations of all 13 compounds were >1.4 μM at 30 min after injection, and 10 compounds had liver concentrations >1.0 μM at 1 h after injection, as measured by LC-MS/MS, with particularly high plasma concentrations obtained with compounds 11 (15.9 μM), 16 (11.6 μM) and 105 (8.7 μM) (Table 5). To evaluate target engagement in vivo, the total and phosphorylated MOB1 levels in livers collected at 1 h post-injection was examined. The analysis revealed a significant reduction (p<0.002) in the levels of Thr35-phosphorylated MOB1 by compounds 11, 16, and 105 compared to the livers of vehicle-treated mice (FIG. 2C, FIG. 2D, and FIG. 2E, Table 6).

TABLE 6
In vivo pharmacokinetic/pharmacodynamic profile of compounds
5, 6, 7, 11, 15, 16, 23, 102, 105, 106, 108, 121, and 119.

| | | | | Relative |
|---|---|---|---|---|
| | | | | decrease |
| | Compound | Plasma | Liver | in liver |
| | dose | concentration | concertation | p-MOB1 |
| Compound | (mg/kg) | (μM) | (μM) | level |
| 5 | 20 | 2.8 | 7.3 | |
| 6 | 20 | 6.9 | 15.3 | 29% |
| 7 | 20 | 6.0 | 14.9 | |
| 11 | 20 | 15.9 | 23.2 | 92%** |
| 15 | 20 | 3.7 | 6.5 | |
| 16 | 20 | 11.6 | 5.9 | 78%** |
| 23 | 20 | 7.1 | 16.6 | 33% |
| 102 | 20 | 1.4 | 15.9 | |
| 105 | 20 | 8.7 | 4.6 | 99%** |
| 106 | 20 | 2.2 | 7.8 | |
| 108 | 20 | 7.3 | 0.9 | |
| 121 | 20 | 1.8 | 0.006 | |
| 119 | 20 | 4.5 | 0.003 | |

**P < 0.002 vs vehicle-administered animals.
Compounds were formulated in 5% DMSO, 10%

Tween 80 and water and were administered to mice by intraperitoneal injection. Blood samples and livers were collected at 30 min and 1 h post-dose, respectively. Plasma concentrations and relative pMOB1 levels were determined by LC-MS/MS and western blot analysis, respectively. (n=3).

Compounds 11, 16, and 105 exhibited a high plasma exposure at 30 min, potent on-target activity in vitro and in vivo, and weak off-target activity against LRRK2, PK/PD characterization of these compounds were performed. The PK properties of 11, 16, and 105 were evaluated by intraperitoneal injection of test compound (10 mg/kg) in mice followed by blood sampling over the next 24 h and quantification of plasma concentrations by LC-MS/MS. Compound 11 exhibited a maximal plasma concentration of 11.9 μM, a time to maximal concentration of 0.5 h, a terminal elimination half-life of 0.7 h, and an area under the curve (AUC) 0-t of 9.5 μmol/L*h (Table 7). Compound 105 had a similar Cₘₐₓ and AUC₀₋ₜ of 11.8 μM and 8.1 μmol/L*h, respectively, while 16 exhibited a lower Cₘₐₓ (5.8 μM) and AUC₀₋ₜ (4.3 μmol/L*h) in mice. The time to maximal concentration for 16 and 105 was 0.25 h, and the half-life was 9.9 and 5.3 h, respectively. In addition, the levels of compound 11, total MOB1, and phosphorylated MOB1 were measured in the mouse livers at 1, 2, and 3 h after intraperitoneal injection of 10 mg/kg. The liver concentrations of 11 were 28.0 μM, 2.5 μM, and 1.2 μM at 1, 2, and 3 h post-dosing, respectively, which correlated well with the maximum inhibition of MOB1 phosphorylation observed at 1 h, followed by slight reductions in the extent of inhibition at 2 and 3 h post-dosing (FIG. 3A, FIG. 3B, and FIG. 3C).

TABLE 7
In vivo pharmacokinetic profile of
compound 11, 16, and 105 in mice.

| | Dose | Cₘₐₓ | Tₘₐₓ | AUC₀₋ₜ | t_{1/2} |
|---|---|---|---|---|---|
| Compound | (mg/kg) | (μM) | (h) | (μmol/L*h) | (h) |
| 11 | 10 | 10.9 | 0.5 | 9.5 | 0.7 |
| 16 | 10 | 5.8 | 0.25 | 4.3 | 9.9 |
| 105 | 10 | 11.8 | 0.25 | 8.1 | 5.3 |

Cₘₐₓ: maximum plasma concentration; Tₘₐₓ: time to Cₘₐₓ; AUC: area under the curve ;. t_{1/2}: terminal half-life.
Compound 11, 16, and 105 was formulated in 5% DMSO, 10% Tween 80, and water and was administered to mice by intraperitoneal injection. Blood samples were collected at 30 min post-injection. Compound 11, 16, and 105 plasma concentration was determined by LC-MS/MS. (n = 3).

To gain further insight into the drug-like properties of the compounds described herein, the solubility, cell permeability and kinase selectivity were evaluated of compound 16. Thus, the kinetic solubility for 16 in PBS was determined to be 189 UM and 16 showed high Caco-2 cell permeability (Table 8 and 9) in cellular efflux assays.

TABLE 8
Kinetic solubility of 6, 11, 16, and 23.

| | Solubility (μM) | | |
|---|---|---|---|
| Compound | Buffer (pH = 7.4) | FaSSIF | FASSGF |
| 6 | 9.2 | 35.7 | 353.0 |
| 11 | 145.0 | 135.5 | 430.0 |
| 16 | 188.5 | 293.5 | 352.5 |
| 23 | <0.6 | 7.0 | 371.5 |

The kinetic solubility was determined for selected compounds 6, 11, 16, and 23 in three media: PBS pH 7.4, FaSSIF, and FaSSGF. Data is represented as an average of a single experiment preformed in duplicate. Atenolol was used as an internal standard.

TABLE 9
Caco-2 permeability of 6, 11, 16, and 23.

| | | Pₐₚₚ (10⁻⁶ cm/s) | | |
|---|---|---|---|---|
| Compound | Permeability | B→A | A→B | Efflux ratio |
| 6 | High | 35.7 | 353.0 | 1.3 |
| 11 | Medium to High | 135.5 | 430.0 | 1.388 |
| 16 | High | 293.5 | 352.5 | 1.3 |
| 23 | High | 7.0 | 371.5 | 1.6 |

The Caco-2 cell permeability was determined for selected compounds 6, 11, 16, and 23. Permeability is classified as follows: high >10, medium, 1-10, and low ≤1. The efflux ratio is calculated by Pₐₚₚ (B→A/A→B). A compound is a potential transporter substrate if efflux ratio >2.0. Atenolol, Propranolol, and Taxol were used as internal standards. Data is represented as an average of a single experiment preformed in duplicate. Apparent permeability=(Pₐₚₚ).

### Example B-11: shRNA-Mediated Knockdown Experiments

MOLM 13 and MV4:11 cells were cultured in Roswell Park Memorial Institute (RPMI) 1640 medium supplemented with 10% FBS and antibiotic/antimycotic (Gibco, Thermo Fisher Scientific). Cells were transfected with SMARTvector Dox-inducible lentiviral small hairpin RNA (shRNA) constructs as follows;
(i) SMARTvector Human Inducible STK4 shRNA: hEF1a, TurboGFP; TCCAATITCCTGAATCACT.
(ii) SMARTvector Human Inducible STK3 shRNA: mCMV, TurboRFP; TGGTGTGGGAATCCAGCTC.
(iii) SMARTvector Inducible Non-targeting Control: hEF1a, Turbo GFP (control for shSTK4).
(iv) SMARTvector Inducible Non-targeting Control: mCMV, TurboRFP (control for shSTK3).

For transfection, 2×10⁶ cells in 2 mL were mixed with the shRNA virus and 8 μg/mL polybrene, incubated for 20 min at room temperature, and centrifuged at room temperature for 1 h at 800×g. The virus-containing medium was removed, and the cells were resuspended in 2 mL fresh culture medium, transferred to 6-well plates, and incubated for 72 h at 37° C. Cells were harvested and centrifuged, and the cell pellet was resuspended in 2 mL fresh medium containing 0.5 μg/mL puromycin and incubated overnight. The next day, the cells were split 1:3 and incubated for 48 h in fresh medium. Stable cell lines were established by selection in 0.2 μg/mL puromycin-containing medium for 1 week.

For analysis of STK expression, cells were incubated with 4 μg/mL doxycycline for 72 h to induce shRNA expression and then analyzed by western blotting as described above. Primary antibodies were: 1:5000 STK4 (rabbit mAb [EP1465Y], ab51134; Abcam: Cambridge, UK), 1:10000 STK3 Thr35 (rabbit mAb [EP1466Y], ab52641: Abcam), and 1:5000 GAPDH (rabbit mAb #2118. Cell Signaling Technology). Secondary antibody was 1:5000 mouse anti-rabbit-HRP (Cell Signaling Technology).

For evaluation of MOLM 13 and MV4:11 cell growth cells stably expressing shRNA plasmids were resuspended in growth medium containing 4 μg/mL doxycyline, plated in quadruplicate at 103 cells/100 μL/well in 96-well plates (Greiner bio-one #655098), and incubated at 37° C. for 0, 2, 4, 6, or 8 days. At each time point, cell viability was measured using the CellTiter-Glo assay (Promega #G7571/2/3) according to the manufacturer's recommendations. For every group, the average RLU value for day 0 was used to calculate fold change. To determine the relative decrease in cell proliferation for shSTK4, the average fold change for the shScr was subtracted from both groups for each day. Results were plotted using Prism 8 software, and significance was determined by two-way ANOVA with Fisher's LSD multiple comparison test.

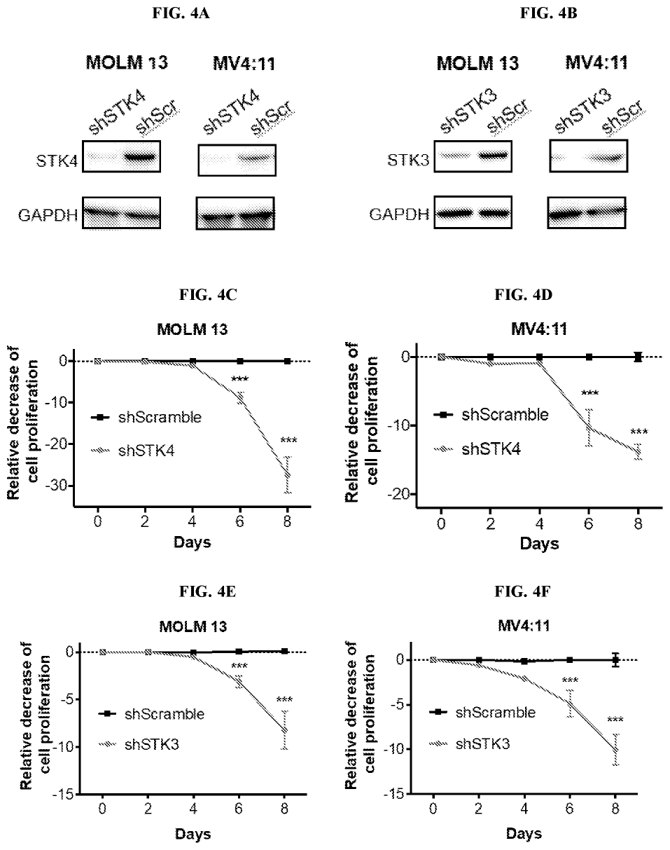

After evaluating the efficacy of the compounds in vitro and showed in vivo target engagement, the compounds potential utility for the treatment of AML were evaluated. In human AML cell lines MOLM 13 and MV4:11, STK3 or STK4 were knocked down by transfection with doxycycline (dox)-inducible control (scrambled) or STK3- or STK4-targeting shRNA constructs. Western blot analysis confirmed effective and specific knockdown of STK3 and STK4 protein expression in both cell lines at 72 h post-induction (FIG. 4A). Transfected cells were selected with puromycin and the effects of STK3/4 knockdown on MOLM 13 and MV4:11 proliferation was determined by measuring cell viability for 8 days after dox-induction of shRNA expression. Significant inhibition of growth was observed at days 6 and 8 after induction of STK3 or STK4 shRNA compared with control shRNA in both cell lines (FIG. 4B). These results demonstrated that these AML cell lines require STK3 and STK4 expression for growth, suggesting that these kinases may be feasible therapeutic targets for AML.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound of Formula (I), or a pharmaceutically acceptable salt thereof: 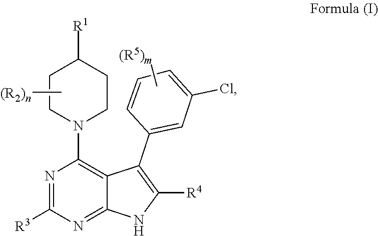
wherein:
R¹ is halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{1a};
each R^{1a} is independently halogen, —CN, —NO₂, —OH, —OR³, —OC(═O)R^{a}, —OC(═O)OR^{b}, —OC(═O)NR^{c}R^{d}, —SH, —SR^{a}, —S(═O) R³, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{1a} on the same atom are taken together to form an oxo;
each R₂ is independently halogen, —CN, —NO₂, —OH, —OR³, —OC(═O)R^{a}, —OC(═O)OR^{b}, —OC(═O)NR^{c}R^{d}, —SH, —SR^{a}, —S(═O)R^{a}, —S(═O) ¿R^{a}, —S(═O)—NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
n is 0-6;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O): C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl.

2. The compound of claim 1, or a pharmaceutically acceptable salt thereof, wherein R³ is hydrogen, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl.

3. The compound of claim 1, or a pharmaceutically acceptable salt thereof, wherein R³ is hydrogen.

4. The compound of claim 1, or a pharmaceutically acceptable salt thereof, wherein R⁴ is hydrogen, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl.

5. The compound of claim 1, or a pharmaceutically acceptable salt thereof, wherein R⁴ is hydrogen.

6. The compound of claim 1, or a pharmaceutically acceptable salt thereof, wherein each R⁵ is independently halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl.

7. The compound of claim 1, or a pharmaceutically acceptable salt thereof, wherein each R⁵ is independently halogen, —CN, —OH, —OR^{a}, —NR^{c}R^{d}, C₁-C₆alkyl, or C₁-C₆haloalkyl.

8. The compound of claim 1, or a pharmaceutically acceptable salt thereof, wherein m is 0 or 1.

9. The compound of claim 1, or a pharmaceutically acceptable salt thereof, wherein m is 0.

10. The compound of claim 1, or a pharmaceutically acceptable salt thereof, wherein each R₂ is independently halogen, C₁-C₆alkyl, or C₁-C₆haloalkyl.

11. The compound of claim 1, or a pharmaceutically acceptable salt thereof, wherein n is 0 or 1.

12. (canceled)

13. The compound of claim 1, or a pharmaceutically acceptable salt thereof, wherein R¹ is halogen, —CN, —OH, —OR^{a}, —OC(═O)R^{a}, —OC(═O)OR^{b}, —OC(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl is optionally and independently substituted with one or more R^{1a}.

14. (canceled)

15. The compound of claim 1, or a pharmaceutically acceptable salt thereof, wherein R¹ is-OH, —OR^{a}, —NR^{c}R^{d}, or —NR^{b}C(═O)R^{a}.

16. The compound of claim 1, wherein the compound is 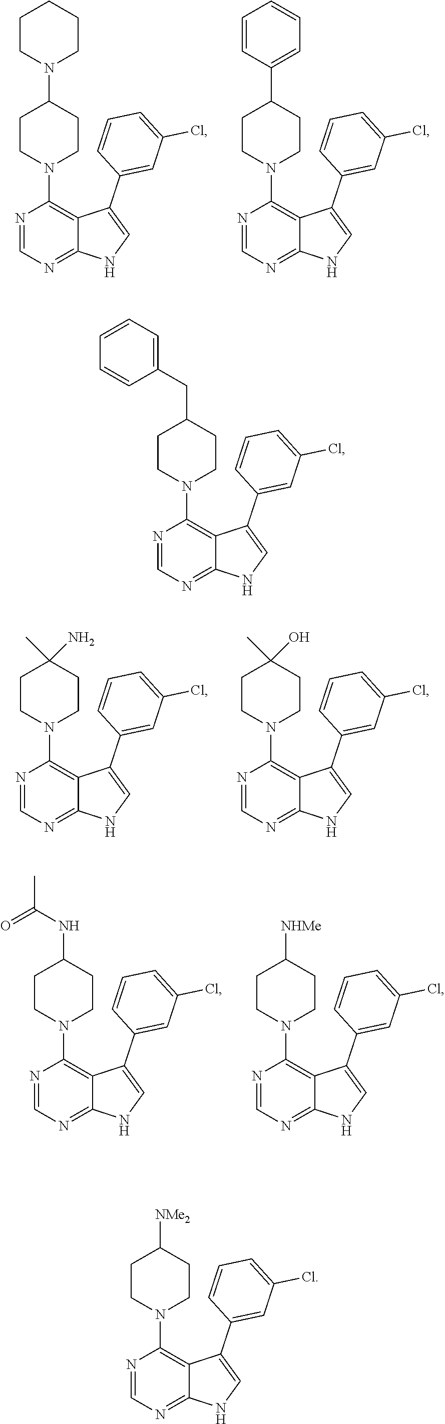
or pharmaceutically acceptable salt thereof.

17. A compound of Formula (II), or a pharmaceutically acceptable salt thereof: 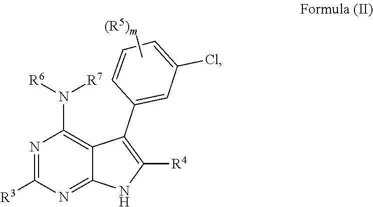
wherein:
R⁶ is hydrogen, C₁-C₆alkyl, C₁-C₆haloalkyl, C₁-C₆hydroxyalkyl, or C₁-C₆aminoalkyl;
R⁷ is C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl; wherein the alkyl, alkenyl, and alkynyl is optionally and independently substituted with one or more R^{7a};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR'S(═O)₂R^{a}, —C(═O)R^{a}, —C(═O)OR^{b}, —C(═O)NR^{c}R^{d}, cycloalkyl, heterocycloalkyl, aryl, or heteroaryl; wherein the cycloalkyl, heterocycloalkyl, aryl, and heteroaryl is optionally and independently substituted with one or more R^{7b};
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl;
or two R^{7b} on the same atom are taken together to form an oxo;
R³ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl): wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O): C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
provided that the compound is not:

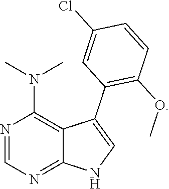 [SMILES: COc1ccc(Cl)cc1-c1c[nH]c2ncnc(N(C)C)c12]

18.-37. (canceled)

38. The compound of claim 17, wherein the compound is 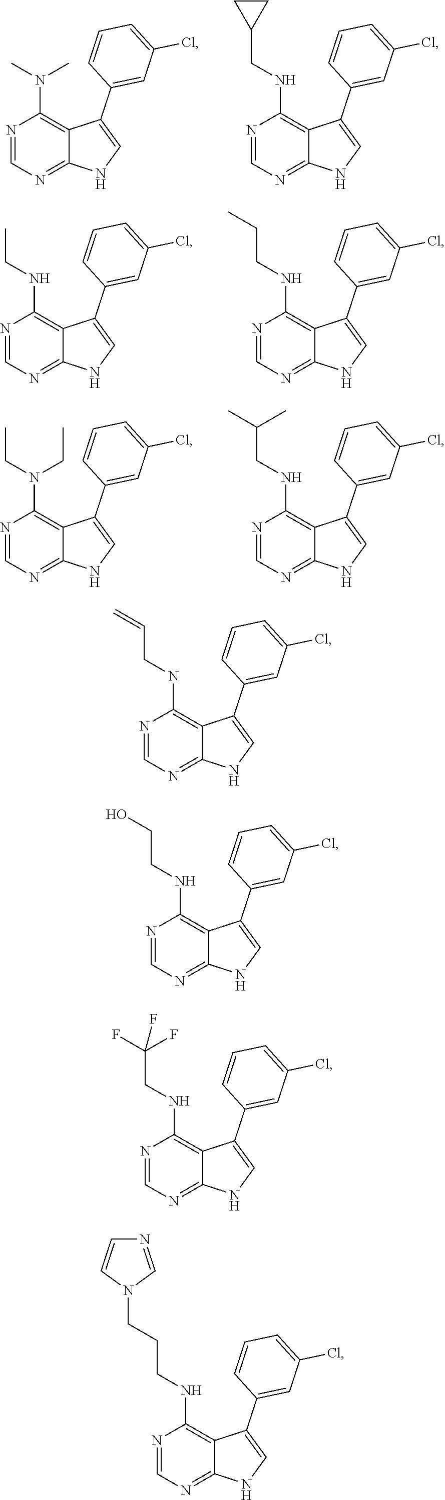 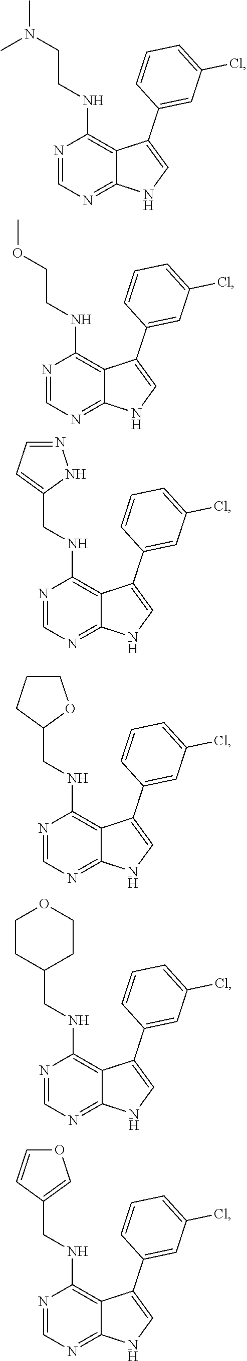 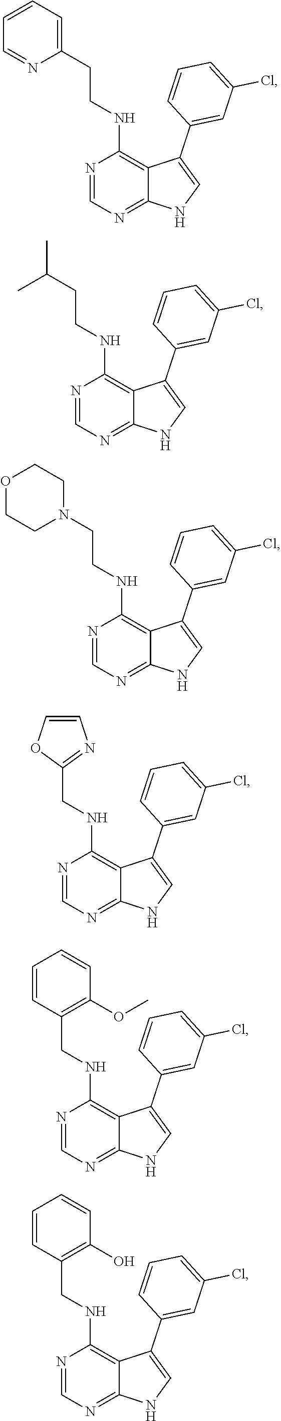 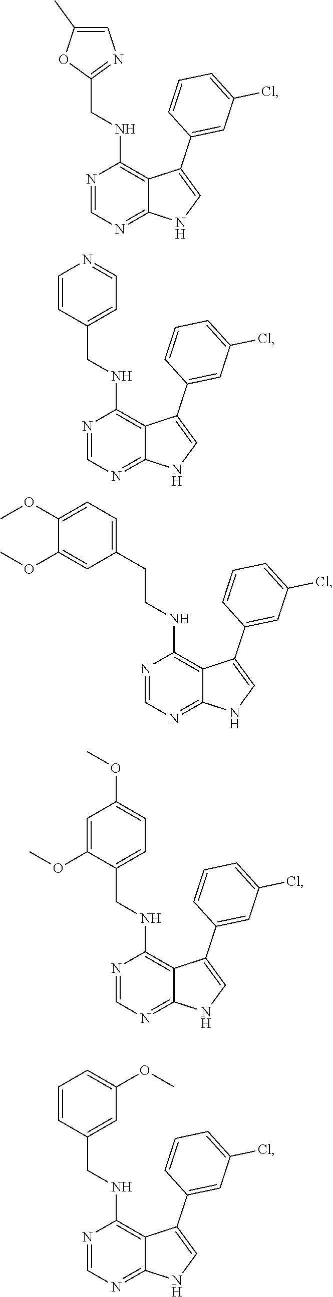 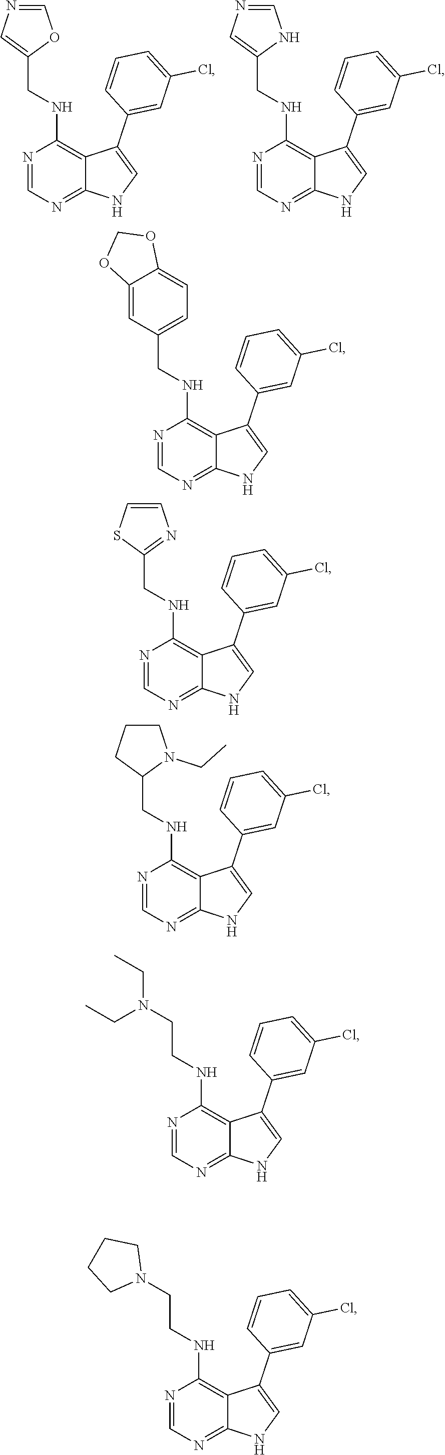 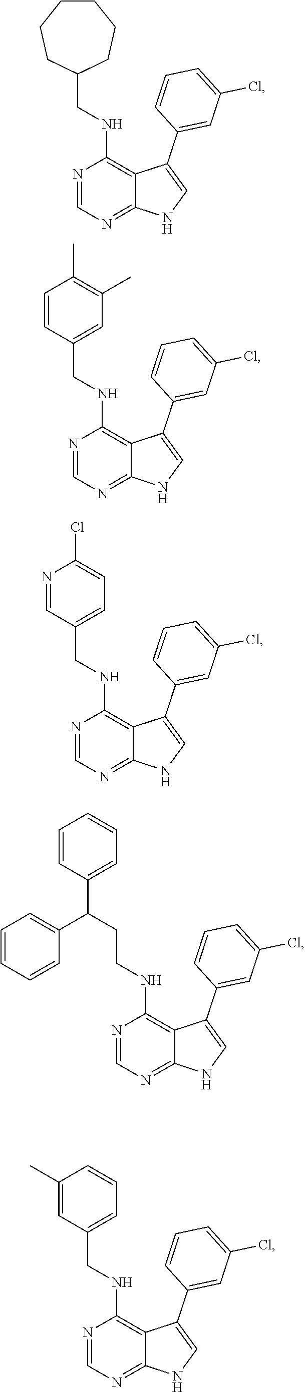 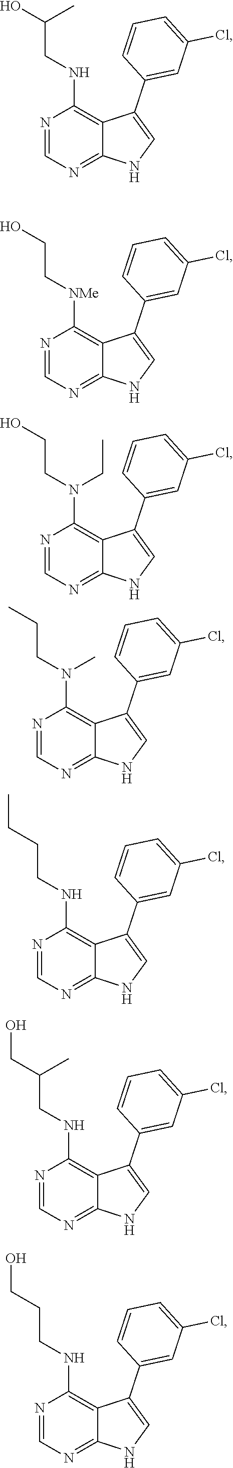 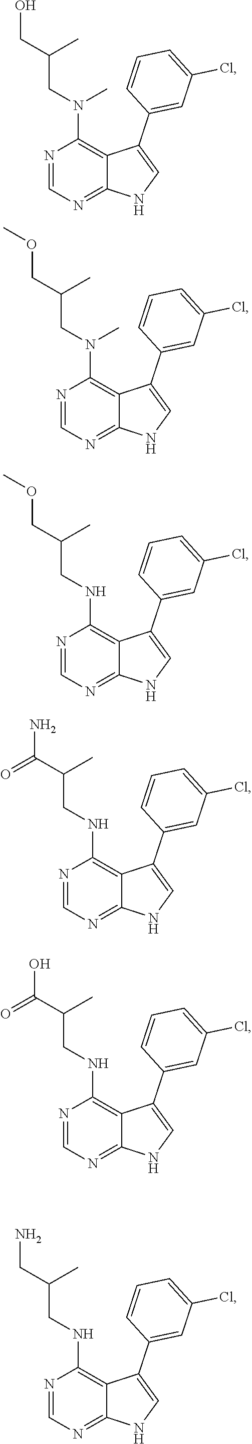 [SMILES: CC(CO)CN(C)c1ncnc2[nH]cc(-c3cccc(Cl)c3)c12 ; COCC(C)CN(C)c1ncnc2[nH]cc(-c3cccc(Cl)c3)c12 ; COCC(C)CNc1ncnc2[nH]cc(-c3cccc(Cl)c3)c12 ; CC(CNc1ncnc2[nH]cc(-c3cccc(Cl)c3)c12)C(N)=O ; CC(CNc1ncnc2[nH]cc(-c3cccc(Cl)c3)c12)C(=O)O ; CC(CN)CNc1ncnc2[nH]cc(-c3cccc(Cl)c3)c12] 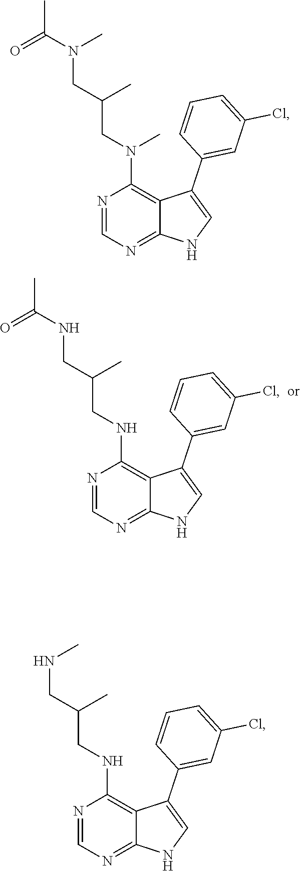
or a pharmaceutically acceptable salt thereof.

39. A compound of Formula (III), or a pharmaceutically acceptable salt thereof: 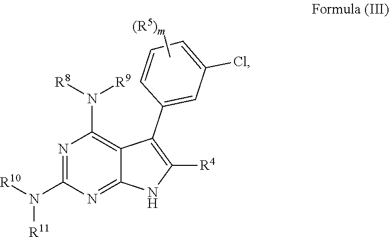
wherein:
R⁸ is hydrogen, C₁-C₆alkyl, C₁-C₆haloalkyl, C₁-C₆hydroxyalkyl, or C₁-C₆aminoalkyl;
R⁹ is hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the cycloalkyl, heterocycloalkyl, aryl, and heteroaryl is optionally and independently substituted with one or more R^{9b};
or two R^{9a} on the same atom are taken together to form an oxo;
each R^{9b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)—NR^{c}R^{d}, —NR^{c}R^{d}, —NR^{b}C(—O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl;
or two R^{9b} on the same atom are taken together to form an oxo;
or R⁸ and R⁹ are taken together with the atom to which they are attached to form a heterocycloalkyl optionally substituted with one or more R^{8a};
each R^{8a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)_NR^{c}R^{d}, —NR^{c}R^{d}, —NR^{b}C(—O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{8a} on the same atom are taken together to form an oxo;
R¹⁰ is hydrogen, C₁-C₆alkyl, or C₁-C₆haloalkyl;
R¹¹ is C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{11a};
each R^{11a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl;
or two R^{11a} on the same atom are taken together to form an oxo;
R⁴ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)_R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
m is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O): C₁-C₆alkyl, —S(═O)₂NH₂, —S(═O)₂NH C₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O) C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, or C₁-C₆aminoalkyl.

40.-67. (canceled)

68. The compound of claim 39, wherein the compound is 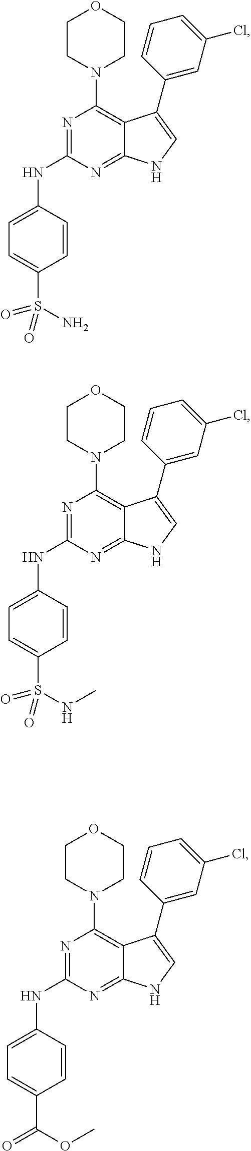 [SMILES: NS(=O)(=O)c1ccc(Nc2nc(N3CCOCC3)c3c(-c4cccc(Cl)c4)c[nH]c3n2)cc1 ; CNS(=O)(=O)c1ccc(Nc2nc(N3CCOCC3)c3c(-c4cccc(Cl)c4)c[nH]c3n2)cc1 ; COC(=O)c1ccc(Nc2nc(N3CCOCC3)c3c(-c4cccc(Cl)c4)c[nH]c3n2)cc1] 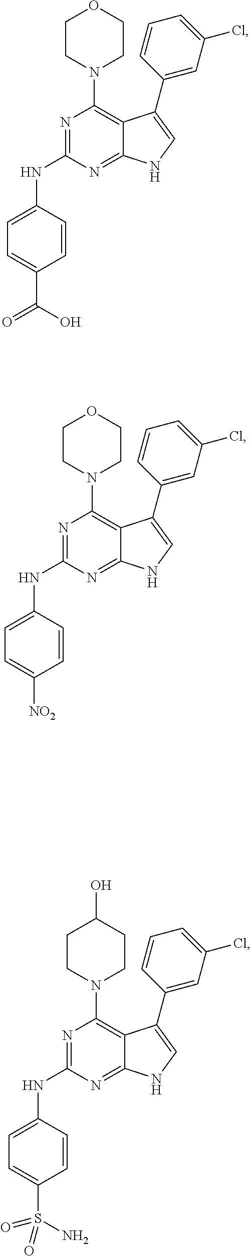 [SMILES: O=C(O)c1ccc(Nc2nc(N3CCOCC3)c3c(-c4cccc(Cl)c4)c[nH]c3n2)cc1 ; O=[N+]([O-])c1ccc(Nc2nc(N3CCOCC3)c3c(-c4cccc(Cl)c4)c[nH]c3n2)cc1 ; NS(=O)(=O)c1ccc(Nc2nc(N3CCC(O)CC3)c3c(-c4cccc(Cl)c4)c[nH]c3n2)cc1] 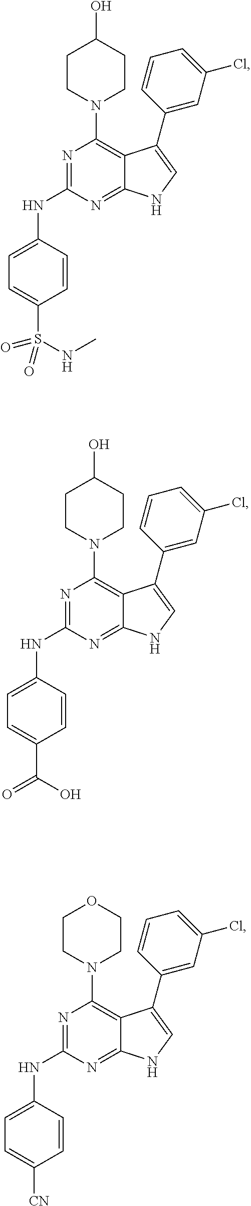 [SMILES: CNS(=O)(=O)c1ccc(Nc2nc(N3CCC(O)CC3)c3c(-c4cccc(Cl)c4)c[nH]c3n2)cc1 ; O=C(O)c1ccc(Nc2nc(N3CCC(O)CC3)c3c(-c4cccc(Cl)c4)c[nH]c3n2)cc1 ; N#Cc1ccc(Nc2nc(N3CCOCC3)c3c(-c4cccc(Cl)c4)c[nH]c3n2)cc1]
or a pharmaceutically acceptable salt thereof.

69. A pharmaceutical composition comprising a compound of claim 1, or a pharmaceutically acceptable salt thereof, and a pharmaceutically acceptable excipient.

70. A method of modulating the activity serine/threonine protein kinase 3 (STK3) or serine/threonine protein kinase 4 (STK4) or both in a mammal, comprising administering to the mammal a compound of claim 1, or a pharmaceutically acceptable salt thereof.

71.-121. (canceled)
